(19) United States
(12) Patent Application Publication
(10) Pub. No.: US 2023/0114811 A1
(43) Pub. Date: Apr. 13, 2023

(54) GLYCOSYLTRANSFERASES, POLYNUCLEOTIDES ENCODING THESE AND METHODS OF USE

(71) Applicant: THE NEW ZEALAND INSTITUTE FOR PLANT AND FOOD RESEARCH LIMITED, Auckland (NZ)

(72) Inventors: Yule WANG, Xi'an City (CN); Ross Graham ATKINSON, Auckland (NZ); Yar-Khing YAUK, Auckland (NZ); Pengmin LI, Yangling (CN)

(73) Assignee: THE NEW ZEALAND INSTITUTE FOR PLANT AND FOOD RESEARCH LIMITED, Auckland (NZ)

(21) Appl. No.: 17/904,632

(22) PCT Filed: Feb. 19, 2021
(86) PCT No.: PCT/IB2021/051407

§ 371 Date: Aug. 19, 2022

(30) Foreign Application Priority Data
Feb. 19, 2020 (NZ) 761877

(51) Int. Cl. C12N 15/82; C12P 19/56; C12N 9/10; C07H 15/203

(52) U.S. Cl. CPC C12N 15/8245; C12P 19/56; C12Y 204/01286; C12N 9/1051; C12N 9/1037; C12Y 203/01074; C07H 15/203

(57) ABSTRACT

The invention provides a method of producing a host cell, plant cell or plant with increased trilobatin content or increased N 4′-O-glycosyltransferase activity, the method comprising transformation of the host cell or plant cell with a polynucleotide encoding a polypeptide with 4′-O-glycosyltransferase activity. The invention also provides host cells, plant cells and plants, genetically modified to contain and or express the polynucleotides.

### FIELD OF THE INVENTION

The present invention relates to compositions and methods for producing plants with altered 4′-O-glycosyltransferase activity and/or altered trilobatin content.

### BACKGROUND

Trilobatin is a plant-based sweetener that is reported to be ˜100× sweeter than sucrose (Jia et al., 2008). Trilobatin is found at high levels in the leaves of a range of crabapple (Malus) species including M. trilobata, M. sieboldii and M. toringo (Williams, 1982; Gutierrez et al., 2018b). It is not found in the domesticated apple (M. x domestica), but has been reported at low levels in the leaves of wild Vitis species (Tanaka et al., 1983). Some Lithocarpus species also contain trilobatin and the leaves are used to prepare sweet tea in China (Sun et al., 2015). The potential utility of trilobatin as a sweetener is recognized in many food and beverage formulations e.g. (Jia et al., 2008; WALTON et al., 2013), however its usefulness is limited by its scarcity. Methods for extraction have been documented from a range of tissues (Sun and Zhang, 2017) and following biotransformation of citrus waste (Lei et al., 2018). Biosynthesis of trilobatin in yeast has also been achieved (Eichenberger et al., 2017), but efficient production has been hampered by lack of knowledge of all enzymes in the biosynthetic pathway.

Trilobatin (phloretin-4′-O-glucoside) and phloridzin (phloretin-2′-O-glucoside) are positional isomers of the dihydrochalcone (DHC) phloretin which is produced on a side branch of the phenylpropanoid pathway. The first committed step in the biosynthesis of DHCs can be catalyzed by a double bond reductase (DBR) that converts p-coumaryl-CoA to p-dihydrocoumaryl-CoA (Dare et al., 2013; Yahyaa et al., 2017). The next step involves decarboxylative condensation and cyclisation of p-dihydrocoumaryl-CoA and three units of malonyl-CoA by chalcone synthase (CHS) to produce phloretin (Gosch et al., 2009; Ibdah et al., 2014). The final step in the pathway requires the action of UDP-glycosyltransferases (UGTs) to attach glucose at either the 2′ or 4′ positions of the chalcone A-ring. Another apple DHC, sieboldin (3-hydroxyphloretin-4′-O-glucoside), is also glycosylated at the 4′ position either after the conversion of phloretin to hydroxyphloretin or by conversion of trilobatin directly to sieboldin.

UGTs are typically encoded by large gene families with over 100 genes being described in Arabidopsis (Ross et al., 2001) and over 200 genes in the M. x domestica genome (Caputi et al., 2012). All UGTs contain a conserved Plant Secondary Product Glycosyltransferase (PSPG) motif that binds the UDP moiety of the activated sugar (Li et al., 2001). Although some UGTs can utilize a broad range of acceptor substrates (Hsu et al., 2017; Yauk et al., 2014), others have been shown to be highly specific (Fukuchi-Mizutani et al., 2003; Jugdé et al., 2008). Systematic classification can facilitate the identification of some UGT activities; however, functionality is generally difficult to ascribe through phylogenetic relatedness alone. In apple, multiple UGTs have been identified that catalyze the 2′-O-glycosylation of phloretin to phloridzin: UGT88F1/MdPGT1 (Jugdé et al., 2008), UGT88F8 (Elejalde-Palmett et al., 2019), UGT88F4, UGT71K1 (Gosch et al., 2010), and UGT71A15 (Gosch et al., 2012). Over-expression of UGT71A15 in transgenic apples did not affect plant morphology or significantly increase phloridzin concentrations, but did increase the molar ratio of phloridzin to phloretin (Gosch et al., 2012). In contrast, UGT88F1 (MdPGT1) knockdown lines showed significantly reduced phloridzin accumulation, severe phenotypic changes, and showed increased resistance to Valsa canker infection (Dare et al., 2017; Zhou et al., 2019).

Two apple enzymes, UGT71A15 and UGT75L17 (MdPh-4′-OGT) that glycosylate phloretin at the 4′ position in vitro have been reported (Gosch et al., 2012; Yahyaa et al., 2016). However, these enzymes are expressed naturally in the leaves and fruit of domesticated apples that produce only phloridzin. As yet, the biosynthetic pathway leading to trilobatin in planta has not been fully resolved.

It would be beneficial to have a means to increase trilobatin levels in plants. It would also be beneficial to have a means to produce trilobatin.

It is an object of at least the preferred embodiments of the present invention to provide compositions and methods for modulating 4′-O-glycosyltransferase activity and/or trilobatin content in plants, yeast, and/or bacteria, and/or to at least provide the public with a useful choice.

In this specification where reference has been made to patent specifications, other external documents, or other sources of information, this is generally for the purpose of providing a context for discussing the features of the invention. Unless specifically stated otherwise, reference to such external documents or such sources of information is not to be construed as an admission that such documents or such sources of information, in any jurisdiction, are prior art or form part of the common general knowledge in the art.

### SUMMARY OF THE INVENTION

In a first aspect, the present invention broadly consists in a method of producing a plant cell or plant with increased trilobatin content, the method comprising transformation of a plant cell with a polynucleotide encoding a polypeptide with the amino acid sequence of any one of SEQ ID NO: 1 to 9, or a variant of the polypeptide.

In one embodiment, there is provided a method of producing a plant cell or plant with increased 4′-O-glycosyltransferase activity, the method comprising transformation of a plant cell with a polynucleotide encoding a polypeptide with the amino acid sequence of any one of SEQ ID NO: 1 to 9, or a variant of the polypeptide.

In preferred embodiments, the variant has 4′-O-glycosyltransferase activity.

Preferably, the variant has at least 70% sequence identity to a polypeptide with the amino acid sequence of any one of SEQ ID NO: 1 to 9.

More preferably, the polynucleotide encodes a polypeptide with an amino acid sequence that has at least 85% identity to the sequence of SEQ ID NO: 1.

Most preferably, the polynucleotide encodes a polypeptide with the amino acid sequence of SEQ ID NO: 1.

In another embodiment, there is provided a method of producing a plant cell or plant with increased trilobatin content, the method comprising transformation of a plant cell with a polynucleotide comprising a nucleotide sequence selected from any one of the sequences SEQ ID NO: 10 to 18, or a variant thereof.

In another embodiment, there is provided a method of producing a plant cell or plant with increased 4′-O-glycosyltransferase activity, the method comprising transformation of a plant cell with a polynucleotide comprising a nucleotide sequence selected from any one of the sequences SEQ ID NO: 10 to 18, or a variant thereof.

In preferred embodiments, the variant encodes a polypeptide that has 4′-O-glycosyltransferase activity.

Preferably, the variant comprises a sequence that has at least 70% sequence identity to the nucleotide sequence of any one of SEQ ID NO: 10 to 18.

More preferably, the variant comprises a sequence that has at least 85% sequence identity to the nucleotide sequence of SEQ ID NO: 10.

Most preferably, the polynucleotide comprises the sequence of SEQ ID NO: 10.

In another embodiment, there is provided a method of producing a plant cell or plant with increased trilobatin content or increased 4′-O-glycosyltransferase activity, the method comprising upregulating in the plant cell or plant expression of a polypeptide with the amino acid sequence of any one of SEQ ID NO: 1 to 9, or a variant of the polypeptide. In another embodiment, there is provided a method of producing a plant cell or plant with increased trilobatin content or increased 4′-O-glycosyltransferase activity, the method comprising upregulating in the plant cell or plant expression of a polynucleotide comprising a nucleotide sequence selected from any one of the sequences SEQ ID NO: 10 to 18, or a variant thereof.

According to some embodiments, the upregulating comprises genetic engineering.

According to some embodiments, the upregulating comprises crossing with a plant which expresses a polypeptide comprising an amino acid sequence having at least 70% sequence identity to a polypeptide with the amino acid sequence of any one of SEQ ID NO: 1 to 9.

According to some embodiments, the upregulating comprises crossing with a plant which expresses a polynucleotide comprising a nucleotide sequence selected from any one of the sequences SEQ ID NO: 10 to 18.

In certain embodiments, the plant cell or plant comprises or is also transformed with a polynucleotide encoding a chalcone synthase (CHS), or a chalcone synthase (CHS) and a double bond reductase (DBR).

Suitable chalcone synthases include HaCHS (NCBI protein accession no: Q9FUB7.1) and HvCHS2 (NCBI protein accession no: Q96562.1), more preferably HaCHS. Suitable double bond reductases include ScTSC13 (NCBI protein accession no: NP_010269.1) and KITSC13 (NCBI protein accession XP_452392.1), more preferably ScTSC13.

In another embodiment, there is provided a genetic construct comprising a polynucleotide encoding a polypeptide with the amino acid sequence of any one of SEQ ID NO: 1 to 9 or a variant of the polypeptide.

In another embodiment, there is provided a genetic construct comprising a polynucleotide comprising a nucleotide sequence selected from any one of the sequences SEQ ID NO: 10 to 18 or a variant thereof.

Preferably, the genetic construct further comprises a polynucleotide encoding a chalcone synthase (CHS) and/or a double bond reductase (DBR) as herein disclosed.

In another embodiment, there is provided a host cell genetically modified to express a polynucleotide encoding a polypeptide with the amino acid sequence of any one of SEQ ID NO: 1 to 9 or a variant of the polypeptide.

In another embodiment, there is provided a host cell genetically modified to express a polynucleotide comprising a nucleotide sequence selected from any one of the sequences SEQ ID NO: 10 to 18 or a variant thereof.

In another embodiment, there is provided a host cell comprising a genetic construct as herein disclosed.

The host cell may be a bacterial, fungal or yeast cell, an insect cell, a plant cell, or a mammalian cell.

In some embodiments, the host cell is a bacterial cell selected from the list consisting of Escherichia, Lactobacillus, Lactococcus, Cornebacterium, Acetobacter, Acinetobacter and Pseudomonas. Preferably the host cell is a facultative anaerobic microorganism, preferably a proteobacterium, in particular an enterobacterium, for example of the genus Escherichia, preferably E. coli, especially E. coli Rosetta, E. coli BL21, E. coli K12, E. coli MG1655, E. coli SE1 and their derivatives.

In some embodiments, the host cell is a yeast cell selected from the list consisting of Saccharomyces cerevisiae, Schizosaccharomyces pombe, Yarrowia lipolytica, Candida glabrata, Ashbya gossypii, Cyberlindnera jadinii, Pichia pastoris, Pichia methanolica, Kluyveromyces lactis, Hansenula polymorpha, Candida boidinii, Arxula adeninivorans, Xanthophyllomyces dendrorhous, and Candida albicans species. In some embodiments, the yeast cell is a Saccharomycete.

In some embodiments, the host cell is a fungal cell selected from the list consisting of Aspergillus spp. and Trichoderma spp.

In another embodiment, there is provided a method for the biosynthesis of trilobatin comprising the steps of culturing a host cell as herein disclosed, capable of expressing a 4′-O-glycosyltransferase, in the presence of phloretin which may be supplied to, or may be present in the host cell.

In some embodiments, UDP-glucose may be supplied to, or may be present in the host cell.

In another embodiment, there is provided a method of producing trilobatin, the method comprising extracting trilobatin from a host cell as herein disclosed.

In another embodiment, there is provided a plant cell genetically modified to express a polynucleotide encoding a polypeptide with the amino acid sequence of any one of SEQ ID NO: 1 to 9 or a variant of the polypeptide.

In another embodiment, there is provided a plant cell genetically modified to express a polynucleotide comprising a nucleotide sequence selected from any one of the sequences SEQ ID NO: 10 to 18 or a variant thereof.

In another embodiment, there is provided a plant that comprises a plant cell as herein disclosed.

In another embodiment, there is provided a method for selecting a plant with altered 4′-O-glycosyltransferase activity, the method comprising testing a plant for altered expression of a polynucleotide encoding a polypeptide with the amino acid sequence of any one of SEQ ID NO: 1 to 9 or a variant of the polypeptide.

In another embodiment, there is provided a method for selecting a plant with altered 4′-O-glycosyltransferase activity, the method comprising testing a plant for altered expression of a polynucleotide comprising a nucleotide sequence selected from any one of the sequences SEQ ID NO: 10 to 18 or a variant thereof.

In another embodiment, there is provided a method for selecting a plant with altered trilobatin content, the method comprising testing a plant for altered expression of a polynucleotide encoding a polypeptide with the amino acid sequence of any one of SEQ ID NO: 1 to 9 or a variant of the polypeptide.

In another embodiment, there is provided a method for selecting a plant with altered trilobatin content, the method comprising testing a plant for altered expression of a polynucleotide comprising a nucleotide sequence selected from any one of the sequences SEQ ID NO: 10 to 18 or a variant thereof.

In another embodiment, there is provided a plant cell or plant produced by a method of producing a plant cell or plant with increased trilobatin content or increased 4′-O-glycosyltransferase activity as herein disclosed.

In another embodiment, there is provided a plant cell or plant selected by a method for selecting a plant with altered trilobatin content or altered 4′-O-glycosyltransferase activity as herein disclosed.

In another embodiment, there is provided a group or population of plants produced by any one of the methods as herein disclosed.

In another embodiment, there is provided a method of producing trilobatin, the method comprising extracting trilobatin from a plant cell or plant having altered trilobatin content or altered 4′-O-glycosyltransferase activity as herein disclosed.

In another embodiment, there is provided a method of producing trilobatin, the method comprising contacting phloretin with UDP-glucose and the expression product of an expression construct encoding a polypeptide with the amino acid sequence of any one of SEQ ID: NO 1 to 9 or a variant of the polypeptide, or a polynucleotide comprising a nucleotide sequence selected from any one of the sequences SEQ ID NO: 10 to 18 or a variant thereof, to obtain trilobatin.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

Although the present invention is broadly as defined above, those persons skilled in the art will appreciate that the invention is not limited thereto and that the invention also includes embodiments of which the following description gives examples.

### BRIEF DESCRIPTION OF THE DRAWINGS

The present invention will now be described with reference to the accompanying drawings in which:

FIG. 1 shows genetic mapping of trilobatin production in a ‘Royal Gala’ x Y3 segregating population. (A) The Trilobatin locus was mapped near the base of LG7 of Y3 using the IRSC 8K SNP array (Chagné et al., 2012). Genetic locations in centiMorgan (cM) are shown on the left and physical location in base pairs on the right (based on the ‘Golden Delicious’ doubled-haploid assembly GDDH13 v1.1). The physical locations of three HRM-SNP markers (Table 1) are indicated. (B) The genomic region of the Trilobatin locus in the ‘Golden Delicious’ v1.0p assembly (top) and the doubled-haploid assembly GDDH13 v1.1 (bottom). The physical positions of two UDP-glucosyltransferase genes identified at the locus in each assembly are shown below the gene model. The black gene model corresponds to PGT2 and the speckled model to PGT3.

FIG. 2 shows activity-directed purification of 4′-oGT activity from flowers of the crabapple hybrid ‘Adams’. Active fractions are shown as dark grey bars. (A) Purification by Q-sepharose chromatography. (B) Purification by phenyl sepharose chromatography using pooled fractions from Q-sepharose. (C) Purification by Superdex 75 chromatography using pooled fractions from phenyl sepharose. Protein concentration (280 nm), enzyme activity, pooled fractions and NaCl or (NH₄)₂SO₄ gradient in the elution buffer are indicated. (D) SDS-PAGE analysis of the four active fractions after purification by Superdex 75 chromatography are shown in lanes 1-4. M=Premixed Broad protein marker (Takara, Dalian, China). Arrow indicates the band sent for LC-MS/MS analysis.

FIG. 3 shows activity-directed purification of 2′-oGT activity from Malus micromalus ‘Makino’ flower petals. After purification by Superdex 75 chromatography (A), three protein fractions with different 2′-O-GT enzyme activities (grey bars) were analyzed by SDS-PAGE (B), lanes 1-3. M=Premixed Broad protein marker (Takara, Dalian, China). Arrow indicates the band sent for LC-MS/MS analysis.

FIG. 4 shows expression and biochemical analysis of PGT1-3. Expression of PGT2 (A), PGT3 (B) and PGT1 (C) were analyzed by qRT-PCR using gene-specific primers (Table 2) in three Malus accessions containing only trilobatin (black bars), three containing trilobatin and phloridzin (white bars), and three containing only phloridzin (grey bars). RG=‘Royal Gala’. Data are means (+SE) of three biological replicates. Expression is presented relative to M. x domestica ‘Fuji’ in (A) and (B) and to M. toringoides in (C) (values set as 1). The products formed by recombinant PGT2 from M. toringodies (E), PGT3 from M. sieboldii (F), PGT1 from M. x domestica ‘Fuji’ (G) and an empty vector control (H) in the presence phloretin and UDP-glucose were analyzed by HPLC and compared to authentic standards (D). P=phloridzin, T=trilobatin and Pt=phloretin.

FIG. 5 shows an SDS-PAGE of recombinant PGT2 (lanes 1-6) and PGT1 (lanes 8-13) purified by Ni²⁺ affinity chromatography. Crude enzyme fractions of PGT2 eluted with 40 mM imidazole buffer (lanes 1, 2) and four purified fractions with 250 mM imidazole buffer (lanes 3-6). Four purified fractions of PGT1 eluted with 250 mM imidazole buffer (lanes 8-11), and crude enzyme fractions (lanes 12, 13) eluted with 40 mM imidazole buffer. M=Premixed Broad protein marker (Takara, Dalian, China). Arrowheads indicate the purified recombinant PGT2 (left) and PGT1 (right).

FIG. 6 shows an amino acid alignment of PGT2 sequences from Malus. Amino acid sequences were aligned using Geneious (version R10, www.qeneious.com). Underlined is the conserved PSPG motif found in all UFGTs (Li et al., 2001).

FIG. 7 shows an LC-MS/MS analysis of products formed by PGT2. Base peak plots: (A) mixed standard of phloretin (Pt) and trilobatin (T); (B) PGT2+phloretin+UDP-glucose; (C) mixed standard of 3-OH phloretin (3Pt)+sieboldin (S); (D) PGT2+3-OH phloretin+UDP-glucose; Mass spectra: (E) fullscan, MS² and MS³ data for phloretin; (F) fullscan, MS² and MS³ data for trilobatin; (G) fullscan, MS² and MS³ data for 3-OH phloretin; and H) fullscan, MS² and MS³ data for sieboldin.

FIG. 8 shows an LC-MS/MS analysis of reactions containing PGT2, quercetin and UDP-glucose. Base peak plots: (A) mixed standard of quercetin (Q) and quercetin-7-O-glucoside (Q7G); (B) PGT2+quercetin+UDP-glucose; (C) standard of quercetin-3-O-glucoside (Q3G); Mass spectra: (D) fullscan, MS² and MS³ data for quercetin; (E) fullscan, MS² and MS³ data for quercetin-7-O-glucoside; (F) fullscan, MS² and MS³ data for quercetin-3-O-glucoside.

FIG. 9 shows the biochemical properties of recombinant PGT2 and PGT1. Activity of PGT2 (A) and PGT1 (B) were tested at 37° C., over the pH range 4-12, using three buffer systems. The temperature-dependent activity of PGT2 and PGT1 are shown in (C) and (D) respectively. The Kₘ values of UDP-glucose (F) were determined at concentrations from 2-500 μM with a fixed phloretin concentration of 500 μM. All data are means (+SE) of three replicates. Kₘ values were calculated by non-linear regression in Sigmaplot.

FIG. 10 shows the engineering of trilobatin and phloridzin production in tobacco. Nicotiana benthamiana leaves were infiltrated with Agrobacterium suspensions containing pHEX2_PGT2, pHEX2_PGT1 or the negative control pHEX2_GUS (each in combination with pHEX2_MdMyb10, pHEX2_MdCHS, pHEX2_MdDBR+pBIN61-p19). Production of trilobatin and phloridzin were analyzed by Dionex-HPLC 7 d post-infiltration. Experiments were performed in triplicate and a single representative trace is shown. (A) pHEX2_PGT2; (B) trilobatin [T] standard; (C) pHEX2_PGT1; (D) phloridzin [P] standard; (E) negative control pHEX2_GUS.

FIG. 11 shows PGT2 expression levels and dihydrochalcone content in transgenic ‘GL3’ apple lines. (A) Relative expression of PGT2 in fourteen transgenic ‘GL3’ lines (#) determined by qRT-PCR using RNA extracted from young leaves. Expression was corrected against Mdactin and is given relative to the wildtype (WT) ‘GL3’ control (value set at 1). (B) DHC content determined by HPLC. Data are presented as mean±SE, n=3 biological replicates. Statistical analysis was performed in GraphPad Prism using Dunnett's Multiple Comparison Test vs WT. No significant differences in phloridzin or phloretin content were observed. Significantly higher PGT2 expression and trilobatin content vs control are shown at P<0.001=***, P<0.01=**, P<0.05=*, ns= not significant.

FIG. 12 shows HPLC and qRT-PCR analysis of transgenic ‘GL3’ plants over-expressing PGT2. (A) Total content of phloridzin (P)+trilobatin (T) in wildtype (WT) ‘GL3’ and each transgenic line (#). Relative expression of PGT1 (B), MdCHS (C), and PGT3 (D) in fourteen transgenic ‘GL3’ apple lines was determined by qRT-PCR using RNA extracted from young leaves. Expression was corrected against Mdactin and is given relative to the wildtype control (value set at 1). Data are presented as mean±SE, n=3. Statistical analysis was performed in GraphPad Prism using Dunnett's Multiple Comparison Test vs WT. No significant differences were observed in total content of P+T content (A), expression of PGT1 (B) or MdCHS (C). For PGT3 expression (D), significantly more than the control at P<0.001=***, P<0.01=**, P<0.05=*, ns= not significant.

FIG. 13 shows dihydrochalcone content in transgenic ‘Royal Gala’ PGT2 over-expression lines. Phenolic compounds were extracted from the young leaves of wildtype (WT) ‘Royal Gala’ and eleven transgenic PGT2 lines (#) into a solution containing 70% methanol and 2% formic acid and dihydrochalcone (DHC) content determined by Dionex-HPLC. (A) Concentration of individual dihydrochalcones in each line. (B) Total content of phloridzin (P) +trilobatin (T) in each line.

FIG. 14 shows an analysis of apple leaf teas and trilobatin isosweetness. (A) Individual phloridzin (P) and trilobatin (T) content in apple leaf tea prepared from wildtype (WT) and two transgenic ‘GL3’ lines over-expressing PGT2 (#1, #9) determined by HPLC. Data are presented as mean±SE, n >7 for dried leaf material (DM) and n=3 for apple leaf teas (LT). Statistical analysis was performed in GraphPad Prism using Dunnett's Multiple Comparison Test vs WT. Significantly higher than WT at P<0.001=***. (B) Isosweetness comparison test between trilobatin and sucrose. Isosweetness was established as 35.2+1.66 (R²=0.98). Data presented are mean±SE, n=5 participants.

FIG. 15 shows the metabolic pathway for producing trilobatin. TAL—tyrosine ammonia lyase; 4CL-4-coumarate-CoA ligase; DBR—double bond reductase; CHS—chalcone synthase; PGT1-phloretin 2′-O-glycosyltransferase 1; PGT2-phloretin 4′-O-glycosyltransferase 2.

FIG. 16 shows the concentration of trilobatin produced by E. coli expressing components of the trilobatin production pathway. ‘ERED+PGT2’ shows the concentration produced by cells expressing TAL, 4CL, CHS2, ERED, and PGT2. ‘ScTSC13+PGT2’ shows the trilobatin concentration produced by cells expressing TAL, 4CL, CHS2, TSC13, and PGT2. C-1′ shows the concentration produced by cells expressing TAL, 4CL, CHS2, and PGT2 but lacking a double-bond reductase. C-2 shows the concentration produced by cells expressing TAL, 4CL and CHS2. C-3 shows the concentration produced by cells expressing TAL and 4CL.

FIG. 17 shows the concentration of trilobatin produced by S. cerevisiae expressing PGT2 at 48 and 72 hours, in a background harbouring HaCHS, ScTSC13, At4CL2, AtPAL2, AmC4H and ScCPR1. No trilobatin production was detected for the phloretin strain control (Pt).

### DETAILED DESCRIPTION

The present invention, in some embodiments thereof, relates to methods of producing trilobatin and for producing host cells including plant cells or plants having increased trilobatin content or increased 4′-O-glycosyltransferase activity.

The present invention is based on the identification, though genetic, biochemical and molecular characterisation described herein, of the stereospecific glycosyltransferase responsible for trilobatin production in planta, phloretin glycosyltransferase 2 (PGT2). Over-expression of PGT2 in domesticated apple leaves significantly increased both trilobatin levels and perceived sweetness of transgenic apple leaf teas.

Identification of the particular glycosyltransferase responsible for trilobatin production allows marker aided selection to be developed to breed plants containing trilobatin, and for high levels of this natural low calorie sweetener to be produced via biotechnological means, such as biopharming in crop plants and metabolic engineering of host cells such as yeast.

Thus, according to one aspect of the present invention there is provided a method of producing a host cell, plant cell or plant with increased trilobatin content or increased 4′-O-glycosyltransferase activity, the method comprising transformation of the host cell or plant cell with a polynucleotide encoding a polypeptide with the amino acid sequence of any one of SEQ ID NO: 1 to 9, or a variant of the polypeptide.

The present invention also provides a method of producing a host cell, plant cell or plant with increased trilobatin content or increased 4′-O-glycosyltransferase activity, the method comprising transformation of the host cell or plant cell with a polynucleotide comprising a nucleotide sequence selected from any one of the sequences SEQ ID NO: 10 to 18, or a variant thereof.

The term “trilobatin” as used herein refers to the dihydrochalcone glycoside also referred to as phloretin-4′-O-glucoside, the structure of which is shown below (Formula I):

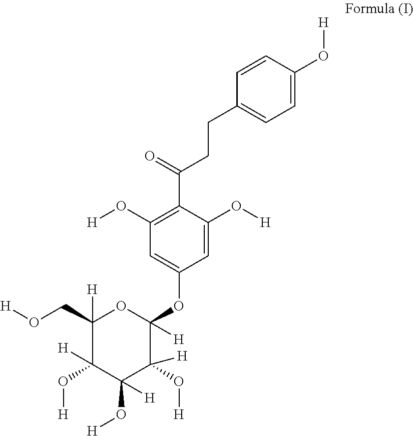 [SMILES: O=C(CCc1ccc(O)cc1)c1c(O)cc(O[C@@H]2O[C@H](CO)[C@@H](O)[C@H](O)[C@H]2O)cc1O]

Trilobatin has the IUPAC name 1-[2,6-dihydroxy-4-[(2S,3R,4S,5S,6R)-3,4,5-trihydroxy-6-(hydroxymethyl)oxan-2-yl]oxyphenyl]-3-(4-hydroxyphenyl)propan-1-one, and CAS #4192-90-9.

The term “phloretin” as used herein refers to the dihydrochalcone also referred to as dihydronaringenin, phloretol or 3-(4-hydroxyphenyl)-1-(2,4,6-trihydroxyphenyl)propan-1-one, the structure of which is shown below (Formula II):

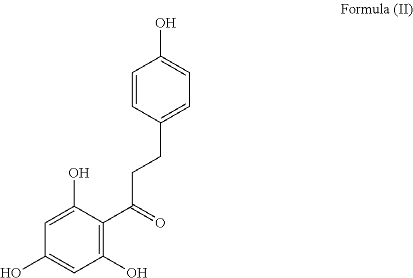 [SMILES: O=C(CCc1ccc(O)cc1)c1c(O)cc(O)cc1O]

The term “having 4′-O-glycosyltransferase activity” as used herein refers to the attachment of a glucose moiety to a substrate at the 4-hydroxyl group.

Trilobatin biosynthesis typically requires the co-substrates phloretin and UDP-glucose and involves attachment of a glucose moiety to phloretin at the 4-hydroxyl group. The attachment of a glucose moiety to phloretin at the 4-hydroxyl group is typically achieved by an enzyme having 4′-O-glycosyltransferase activity. Such a glycosyltransferase enzyme catalyzes the transfer of a saccharide moiety (e.g. glucose) from an activated nucleotide sugar (e.g. UDP-glucose) to the 4-hydroxyl group of the acceptor molecule (e.g. phloretin).

The present inventors have identified the stereospecific glycosyltransferase responsible for trilobatin production in planta, which is termed phloretin glycosyltransferase 2 (PGT2).

The applicants have identified polynucleotides (SEQ ID NOs: 10 to 18) that encode polypeptides (SEQ ID NOs: 1 to 9) that have 4′-O-glycosyltransferase activity, as summarised in Table 12.

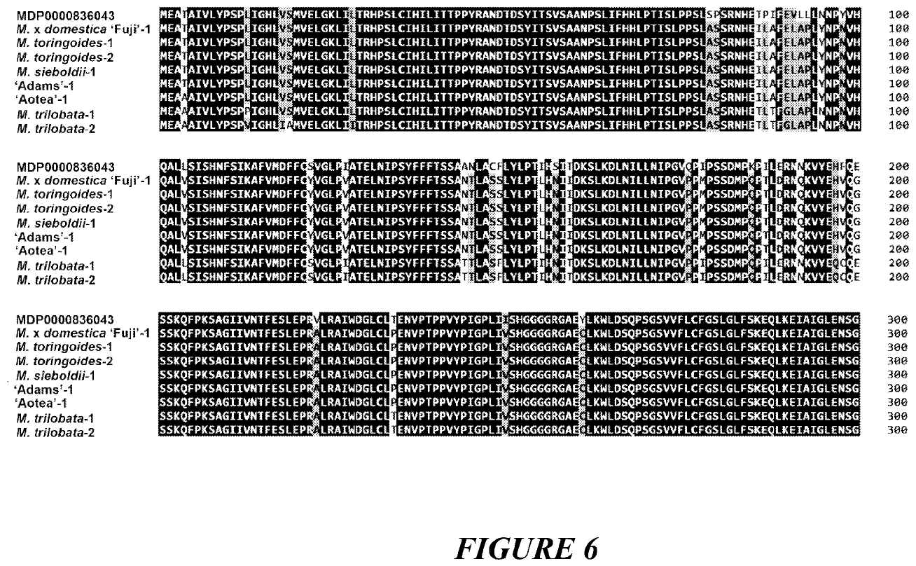

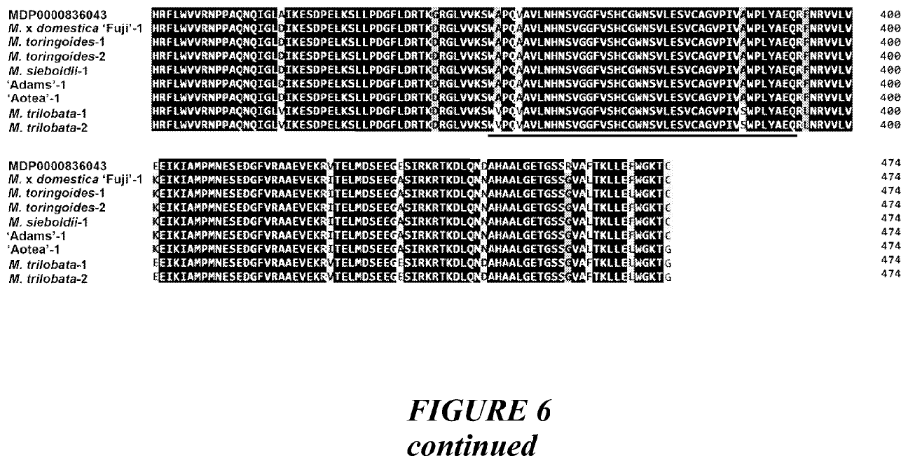

The applicants have shown that all of the 4′-O-glycosyltransferase polypeptide sequences disclosed (SEQ ID NO: 1 to 9) have significant sequence conservation and are variants of one another (FIG. 6).

Similarly the applicants have shown that all of the disclosed 4′-O-glycosyltransferase polynucleotide sequences (SEQ ID NO: 10 to 18) have significant sequence conservation and are variants of one another.

Genetic constructs, vectors and plants containing these polynucleotide sequences (SEQ ID NOs: 10 to 18) or sequences encoding the polypeptide sequences (SEQ ID NO: 1 to 9) are disclosed herein.

In certain embodiments, there are provided plants and host cells comprising the genetic constructs and vectors disclosed herein.

In some embodiments, there are provided plants altered in 4′-O-glycosyltransferase activity, relative to suitable control plants, and plants altered in trilobatin content relative to suitable control plants. In some embodiments, there are provided plants with increased 4′-O-glycosyltransferase activity and increased trilobatin.

In other embodiments there are provided methods for the production of such plants and methods of selection of such plants.

Suitable control plants include non-transformed plants of the same species or variety or plants transformed with control constructs.

Polynucleotides and Fragments

The term “polynucleotide(s),” as used herein, means a single or double-stranded deoxyribonucleotide or ribonucleotide polymer of any length but preferably at least 15 nucleotides, and include as non-limiting examples, coding and non-coding sequences of a gene, sense and antisense sequences complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

A “fragment” of a polynucleotide sequence provided herein is a subsequence of contiguous nucleotides that is capable of specific hybridization to a target of interest, e.g., a sequence that is at least 15 nucleotides in length. Fragments as herein disclosed comprise 15 nucleotides, preferably at least 20 nucleotides, more preferably at least 30 nucleotides, more preferably at least 50 nucleotides, more preferably at least 50 nucleotides and most preferably at least 60 nucleotides of contiguous nucleotides of a polynucleotide as herein disclosed. A fragment of a polynucleotide sequence can be used in antisense, gene silencing, triple helix or ribozyme technology, or as a primer, a probe, included in a microarray, or used in polynucleotide-based selection methods as herein disclosed.

The term “primer” refers to a short polynucleotide, usually having a free 3′OH group, that is hybridized to a template and used for priming polymerization of a polynucleotide complementary to the template. Such a primer is preferably at least 5, more preferably at least 6, more preferably at least 7, more preferably at least 9, more preferably at least 10, more preferably at least 11, more preferably at least 12, more preferably at least 13, more preferably at least 14, more preferably at least 15, more preferably at least 16, more preferably at least 17, more preferably at least 18, more preferably at least 19, more preferably at least 20 nucleotides in length.

The term “probe” refers to a short polynucleotide that is used to detect a polynucleotide sequence, that is complementary to the probe, in a hybridization-based assay. The probe may consist of a “fragment” of a polynucleotide as defined herein. Preferably such a probe is at least 5, more preferably at least 10, more preferably at least 20, more preferably at least 30, more preferably at least 40, more preferably at least 50, more preferably at least 100, more preferably at least 200, more preferably at least 300, more preferably at least 400 and most preferably at least 500 nucleotides in length.

Polypeptides and Fragments

The term “polypeptide”, as used herein, encompasses amino acid chains of any length but preferably at least 5 amino acids, including full-length proteins, in which amino acid residues are linked by covalent peptide bonds. Polypeptides as herein disclosed may be purified natural products, or may be produced partially or wholly using recombinant or synthetic techniques. The term may refer to a polypeptide, an aggregate of a polypeptide such as a dimer or other multimer, a fusion polypeptide, a polypeptide fragment, a polypeptide variant, or derivative thereof.

A “fragment” of a polypeptide is a subsequence of the polypeptide that performs a function that is required for the biological activity and/or provides three dimensional structure of the polypeptide. The term may refer to a polypeptide, an aggregate of a polypeptide such as a dimer or other multimer, a fusion polypeptide, a polypeptide fragment, a polypeptide variant, or derivative thereof capable of performing the above enzymatic activity.

The term “isolated” as applied to the polynucleotide or polypeptide sequences disclosed herein is used to refer to sequences that are removed from their natural cellular environment. An isolated molecule may be obtained by any method or combination of methods including biochemical, recombinant, and synthetic techniques.

The term “recombinant” refers to a polynucleotide sequence that is removed from sequences that surround it in its natural context and/or is recombined with sequences that are not present in its natural context.

A “recombinant” polypeptide sequence is produced by translation from a “recombinant” polynucleotide sequence.

The term “derived from” with respect to polynucleotides or polypeptides as disclosed herein being derived from a particular genera or species, means that the polynucleotide or polypeptide has the same sequence as a polynucleotide or polypeptide found naturally in that genera or species. The polynucleotide or polypeptide, derived from a particular genera or species, may therefore be produced synthetically or recombinantly.

Variants

As used herein, the term “variant” refers to polynucleotide or polypeptide sequences different from the specifically identified sequences, wherein one or more nucleotides or amino acid residues is deleted, substituted, or added. Variants may be naturally occurring allelic variants, or non-naturally occurring variants. Variants may be from the same or from other species and may encompass homologues, paralogues and orthologues. Variants described herein can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered variants.

In certain embodiments, variants of the polynucleotides and polypeptides disclosed herein possess biological activities that are the same or similar to those of the polynucleotides or polypeptides disclosed herein. The term “variant” with reference to polynucleotides and polypeptides encompasses all forms of polynucleotides and polypeptides as defined herein.

Polynucleotide variants Variant polynucleotide sequences preferably exhibit at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequence as disclosed herein.

Identity is found over a comparison window of at least 20 nucleotide positions, preferably at least 50 nucleotide positions, more preferably at least 100 nucleotide positions, more preferably at least 200 nucleotide positions, more preferably at least 300 nucleotide positions, more preferably at least 400 nucleotide positions, more preferably at least 500 nucleotide positions, and most preferably over the entire length of a polynucleotide disclosed herein.

Polynucleotide sequence identity can be determined in the following manner. The subject polynucleotide sequence is compared to a candidate polynucleotide sequence using BLASTN (from the BLAST suite of programs, version 2.2.5 [November 2002]) in bl2seq (Tatiana A. Tatusova, Thomas L. Madden (1999), “Blast 2 sequences—a new tool for comparing protein and nucleotide sequences”, FEMS Microbiol Lett. 174:247-250), which is publicly available from NCBI (ftp://ftp.ncbi.nih.gov/blast/). The default parameters of bl2seq are utilized except that filtering of low complexity parts should be turned off.

The identity of polynucleotide sequences may be examined using the following unix command line parameters:
bl2seq -i nucleotideseq1 -j nucleotideseq2 -F F -p blastn

The parameter -F F turns off filtering of low complexity sections. The parameter -p selects the appropriate algorithm for the pair of sequences. The bl2seq program reports sequence identity as both the number and percentage of identical nucleotides in a line “Identities=” Polynucleotide sequence identity may also be calculated over the entire length of the overlap between a candidate and subject polynucleotide sequences using global sequence alignment programs (e.g. Needleman, S. B. and Wunsch, C. D. (1970) J. Mol. Biol. 48, 443-453). A full implementation of the Needleman-Wunsch global alignment algorithm is found in the needle program in the EMBOSS package (Rice, P. Longden, I. and Bleasby, A. EMBOSS: The European Molecular Biology Open Software Suite, Trends in Genetics June 2000, vol 16, No 6. pp. 276-277) which can be obtained from http://www.hgmp.mrc.ac.uk/Software/EMBOSS/. The European Bioinformatics Institute server also provides the facility to perform EMBOSS-needle global alignments between two sequences online at http:/www.ebi.ac.uk/emboss/align/.

Alternatively the GAP program may be used which computes an optimal global alignment of two sequences without penalizing terminal gaps. GAP is described in the following paper: Huang, X. (1994) On Global Sequence Alignment. Computer Applications in the Biosciences 10, 227-235.

Another method for calculating polynucleotide % sequence identity is based on aligning sequences to be compared using Clustal X (Jeanmougin et al., 1998, Trends Biochem. Sci. 23, 403-5.)

Polynucleotide variants of the present invention also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs described supra.

The similarity of polynucleotide sequences may be examined using the following unix command line parameters:

bl2seq -i nucleotideseq1 -j nucleotideseq2 -F F -p tblastx

The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. The size of this database is set by default in the bl2seq program. For small E values, much less than one, the E value is approximately the probability of such a random match.

Variant polynucleotide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰ more preferably less than 1×10⁻⁷⁰, more preferably less than 1×10⁻⁸⁰, more preferably less than 1×10⁻⁹⁰ and most preferably less than 1×10⁻¹⁰⁰ when compared with any one of the specifically identified sequences.

Alternatively, variant polynucleotides as disclosed herein hybridize to the specified polynucleotide sequences, or complements thereof under stringent conditions.

The term “hybridize under stringent conditions”, and grammatical equivalents thereof, refers to the ability of a polynucleotide molecule to hybridize to a target polynucleotide molecule (such as a target polynucleotide molecule immobilized on a DNA or RNA blot, such as a Southern blot or Northern blot) under defined conditions of temperature and salt concentration. The ability to hybridize under stringent hybridization conditions can be determined by initially hybridizing under less stringent conditions then increasing the stringency to the desired stringency.

With respect to polynucleotide molecules greater than about 100 bases in length, typical stringent hybridization conditions are no more than 25 to 30° C. (for example, 10° C.) below the melting temperature (Tm) of the native duplex (see generally, Sambrook et al., Eds, 1987, Molecular Cloning, A Laboratory Manual, 2nd Ed. Cold Spring Harbor Press; Ausubel et al., 1987, Current Protocols in Molecular Biology, Greene Publishing,). Tm for polynucleotide molecules greater than about 100 bases can be calculated by the formula Tm=81.5+0.41% (G+C-log (Na+). (Sambrook et al., Eds, 1987, Molecular Cloning, A Laboratory Manual, 2nd Ed. Cold Spring Harbor Press; Bolton and McCarthy, 1962, PNAS 84:1390). Typical stringent conditions for polynucleotide of greater than 100 bases in length would be hybridization conditions such as prewashing in a solution of 6×SSC, 0.2% SDS; hybridizing at 65° C., 6×SSC, 0.2% SDS overnight; followed by two washes of 30 minutes each in 1×SSC, 0.1% SDS at 65° C. and two washes of 30 minutes each in 0.2×SSC, 0.1% SDS at 65° C.

With respect to polynucleotide molecules having a length less than 100 bases, exemplary stringent hybridization conditions are 5 to 10° C. below Tm. On average, the Tm of a polynucleotide molecule of length less than 100 bp is reduced by approximately (500/oligonucleotide length) ° C.

With respect to the DNA mimics known as peptide nucleic acids (PNAs) (Nielsen et al., Science. 1991 Dec. 6; 254(5037):1497-500) Tm values are higher than those for DNA-DNA or DNA-RNA hybrids, and can be calculated using the formula described in Giesen et al., Nucleic Acids Res. 1998 Nov. 1; 26(21):5004-6. Exemplary stringent hybridization conditions for a DNA-PNA hybrid having a length less than 100 bases are 5 to 10° C. below the Tm.

Variant polynucleotides as disclosed herein also encompass polynucleotides that differ from the sequences as herein disclosed but that, as a consequence of the degeneracy of the genetic code, encode a polypeptide having similar activity to a polypeptide encoded by a polynucleotide of the present invention. A sequence alteration that does not change the amino acid sequence of the polypeptide is a “silent variation”. Except for ATG (methionine) and TGG (tryptophan), other codons for the same amino acid may be changed by art recognized techniques, e.g., to optimize codon usage in a particular host organism.

Polynucleotide sequence alterations resulting in conservative substitutions of one or several amino acids in the encoded polypeptide sequence without significantly altering its biological activity are also included in the invention. A skilled artisan will be aware of methods for making phenotypically silent amino acid substitutions (see, e.g., Bowie et al., 1990, Science 247, 1306).

Variant polynucleotides due to silent variations and conservative substitutions in the encoded polypeptide sequence may be determined using the publicly available bl2seq program from the BLAST suite of programs (version 2.2.5 [November 2002]) from NCBI (ftp://ftp.ncbi.nih.gov/blast/) via the tblastx algorithm as previously described.

The function of a variant polynucleotide disclosed herein as a 4′-O-glycosyltransferase may be assessed for example by expressing such a sequence in bacteria and testing activity of the encoded protein as described in the Examples section. Function of a variant may also be tested for its ability to alter 4′-O-glycosyltransferase activity or trilobatin content in plants, also as described in the Examples section herein.

Polypeptide Variants

The term “variant” with reference to polypeptides encompasses naturally occurring, recombinantly and synthetically produced polypeptides. Variant polypeptide sequences preferably exhibit at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a sequences of the present invention. Identity is found over a comparison window of at least 20 amino acid positions, preferably at least 50 amino acid positions, more preferably at least 100 amino acid positions, and most preferably over the entire length of a polypeptide as herein disclosed.

Polypeptide sequence identity can be determined in the following manner. The subject polypeptide sequence is compared to a candidate polypeptide sequence using BLASTP (from the BLAST suite of programs, version 2.2.5 [November 2002]) in bl2seq, which is publicly available from NCBI (ftp://ftp.ncbi.nih.gov/blast/). The default parameters of bl2seq are utilized except that filtering of low complexity regions should be turned off.

Polypeptide sequence identity may also be calculated over the entire length of the overlap between a candidate and subject polypeptide sequences using global sequence alignment programs. EMBOSS-needle (available at http:/www.ebi.ac.uk/emboss/align/) and GAP (Huang, X. (1994) On Global Sequence Alignment. Computer Applications in the Biosciences 10, 227-235.) as discussed above are also suitable global sequence alignment programs for calculating polypeptide sequence identity.

Another method for calculating polypeptide % sequence identity is based on aligning sequences to be compared using Clustal X (Jeanmougin et al., 1998, Trends Biochem. Sci. 23, 403-5.) Polypeptide variants as disclosed herein also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [November 2002]) from NCBI (ftp://ftp.ncbi.nih.gov/blast/). The similarity of polypeptide sequences may be examined using the following unix command line parameters:

bl2seq -i peptideseq1 -j peptideseq2 -F F -p blastp The parameter -F F turns off filtering of low complexity sections. The parameter -p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Variant polypeptide sequences preferably exhibit an E value of less than 1×10⁻¹⁰ more preferably less than 1×10⁻²⁰, more preferably less than 1×10⁻³⁰, more preferably less than 1×10⁻⁴⁰, more preferably less than 1×10⁻⁵⁰, more preferably less than 1×10⁻⁶⁰ more preferably less than 1×10⁻⁷⁰, more preferably less than 1×10⁻⁸⁰, more preferably less than 1×10⁻⁹⁰ and most preferably less than 1×10⁻¹⁰⁰ when compared with any one of the specifically identified sequences.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

Methods of assaying 4′-O-glycosyltransferase activity are well known in the art and include, for example, standard glycosyltransferase enzyme assay for LC-MS and radioactive assay for the enzyme UDP-glucose pyrophosphorylase. The function of a polypeptide variant as a 4′-O-glycosyltransferase may also be assessed by the methods described in the Examples section herein.

Methods for Identifying Variants

Physical Methods

Variant polypeptides may be identified using PCR-based methods (Mullis et al., Eds. 1994 The Polymerase Chain Reaction, Birkhauser). Typically, the polynucleotide sequence of a primer, useful to amplify variants of polynucleotide molecules as disclosed herein by PCR, may be based on a sequence encoding a conserved region of the corresponding amino acid sequence.

Alternatively library screening methods, well known to those skilled in the art, may be employed (Sambrook et al., Molecular Cloning: A Laboratory Manual, 2nd Ed. Cold Spring Harbor Press, 1987). When identifying variants of the probe sequence, hybridization and/or wash stringency will typically be reduced relatively to when exact sequence matches are sought.

Polypeptide variants may also be identified by physical methods, for example by screening expression libraries using antibodies raised against polypeptides disclosed herein (Sambrook et al., Molecular Cloning: A Laboratory Manual, 2nd Ed. Cold Spring Harbor Press, 1987) or by identifying polypeptides from natural sources with the aid of such antibodies.

Computer Based Methods

The variant sequences as disclosed herein, including both polynucleotide and polypeptide variants, may also be identified by computer-based methods well-known to those skilled in the art, using public domain sequence alignment algorithms and sequence similarity search tools to search sequence databases (public domain databases include Genbank, EMBL, Swiss-Prot, PIR and others). See, e.g., Nucleic Acids Res. 29: 1-10 and 11-16, 2001 for examples of online resources. Similarity searches retrieve and align target sequences for comparison with a sequence to be analyzed (i.e., a query sequence). Sequence comparison algorithms use scoring matrices to assign an overall score to each of the alignments.

An exemplary family of programs useful for identifying variants in sequence databases is the BLAST suite of programs (version 2.2.5 [November 2002]) including BLASTN, BLASTP, BLASTX, tBLASTN and tBLASTX, which are publicly available from (ftp://ftp.ncbi.nih.gov/blast/) or from the National Center for Biotechnology Information (NCBI), National Library of Medicine, Building 38A, Room 8N805, Bethesda, Md. 20894 USA. The NCBI server also provides the facility to use the programs to screen a number of publicly available sequence databases. BLASTN compares a nucleotide query sequence against a nucleotide sequence database. BLASTP compares an amino acid query sequence against a protein sequence database. BLASTX compares a nucleotide query sequence translated in all reading frames against a protein sequence database. tBLASTN compares a protein query sequence against a nucleotide sequence database dynamically translated in all reading frames. tBLASTX compares the six-frame translations of a nucleotide query sequence against the six-frame translations of a nucleotide sequence database. The BLAST programs may be used with default parameters or the parameters may be altered as required to refine the screen.

The use of the BLAST family of algorithms, including BLASTN, BLASTP, and BLASTX, is described in the publication of Altschul et al., Nucleic Acids Res. 25: 3389-3402, 1997.

The “hits” to one or more database sequences by a queried sequence produced by BLASTN, BLASTP, BLASTX, tBLASTN, tBLASTX, or a similar algorithm, align and identify similar portions of sequences. The hits are arranged in order of the degree of similarity and the length of sequence overlap. Hits to a database sequence generally represent an overlap over only a fraction of the sequence length of the queried sequence.

The BLASTN, BLASTP, BLASTX, tBLASTN and tBLASTX algorithms also produce “Expect” values for alignments. The Expect value (E) indicates the number of hits one can “expect” to see by chance when searching a database of the same size containing random contiguous sequences. The Expect value is used as a significance threshold for determining whether the hit to a database indicates true similarity. For example, an E value of 0.1 assigned to a polynucleotide hit is interpreted as meaning that in a database of the size of the database screened, one might expect to see 0.1 matches over the aligned portion of the sequence with a similar score simply by chance. For sequences having an E value of 0.01 or less over aligned and matched portions, the probability of finding a match by chance in that database is 1% or less using the BLASTN, BLASTP, BLASTX, tBLASTN or tBLASTX algorithm.

Multiple sequence alignments of a group of related sequences can be carried out with CLUSTALW (Thompson, J. D., Higgins, D. G. and Gibson, T. J. (1994) CLUSTALW: improving the sensitivity of progressive multiple sequence alignment through sequence weighting, positions-specific gap penalties and weight matrix choice. Nucleic Acids Research, 22:4673-4680, http://www-igbmc.u-strasbg.fr/BioInfo/ClustalW/Top.html) or T-COFFEE (Cedric Notredame, Desmond G. Higgins, Jaap Heringa, T-Coffee: A novel method for fast and accurate multiple sequence alignment, J. Mol. Biol. (2000) 302: 205-217)) or PILEUP, which uses progressive, pairwise alignments. (Feng and Doolittle, 1987, J. Mol. Evol. 25, 351).

Pattern recognition software applications are available for finding motifs or signature sequences. For example, MEME (Multiple Em for Motif Elicitation) finds motifs and signature sequences in a set of sequences, and MAST (Motif Alignment and Search Tool) uses these motifs to identify similar or the same motifs in query sequences. The MAST results are provided as a series of alignments with appropriate statistical data and a visual overview of the motifs found. MEME and MAST were developed at the University of California, San Diego.

PROSITE (Bairoch and Bucher, 1994, Nucleic Acids Res. 22, 3583; Hofmann et al., 1999, Nucleic Acids Res. 27, 215) is a method of identifying the functions of uncharacterized proteins translated from genomic or cDNA sequences. The PROSITE database (www.expasy.org/prosite) contains biologically significant patterns and profiles and is designed so that it can be used with appropriate computational tools to assign a new sequence to a known family of proteins or to determine which known domain(s) are present in the sequence (Falquet et al., 2002, Nucleic Acids Res. 30, 235). Prosearch is a tool that can search SWISS-PROT and EMBL databases with a given sequence pattern or signature.

Another example of a protein domain model database is Pfam (Sonnhammer et al., 1997, A comprehensive database of protein families based on seed alignments, Proteins, 28: 405-420; Finn et al., 2010, The Pfam protein families database', Nucl. Acids Res., 38: D211-D222). “Pfam” refers to a large collection of protein domains and protein families maintained by the Pfam Consortium and available at several sponsored world wide web sites, including: pfam.xfam.org/ (European Bioinformatics Institute (EMBL-EBI). The latest release of Pfam is Pfam 30.0 (June 2016). Pfam domains and families are identified using multiple sequence alignments and hidden Markov models (HMMs). Pfam-A family or domain assignments, are high quality assignments generated by a curated seed alignment using representative members of a protein family and profile hidden Markov models based on the seed alignment. (Unless otherwise specified, matches of a queried protein to a Pfam domain or family are Pfam-A matches.) All identified sequences belonging to the family are then used to automatically generate a full alignment for the family (Sonnhammer (1998) Nucleic Acids Research 26, 320-322; Bateman (2000) Nucleic Acids Research 26, 263-266; Bateman (2004) Nucleic Acids Research 32, Database Issue, D138-D141; Finn (2006) Nucleic Acids Research Database Issue 34, D247-251; Finn (2010) Nucleic Acids Research Database Issue 38, D21 1-222). By accessing the Pfam database, for example, using the above-referenced website, protein sequences can be queried against the HMMs using HMMER homology search software {e.g., HMMER2, HMMER3, or a higher version, hmmer.org). Significant matches that identify a queried protein as being in a pfam family (or as having a particular Pfam domain) are those in which the bit score is greater than or equal to the gathering threshold for the Pfam domain. Expectation values (e values) can also be used as a criterion for inclusion of a queried protein in a Pfam or for determining whether a queried protein has a particular Pfam domain, where low e values (much less than 1.0, for example less than 0.1, or less than or equal to 0.01) represent low probabilities that a match is due to chance.

The function of a variant polynucleotide as disclosed herein as encoding 4′-O-glycosyltransferases can be tested for the activity, or can be tested for their capability to alter trilobatin content in plants by methods described in the Examples section herein.

Methods for Isolating or Producing Polynucleotides

The polynucleotide molecules disclosed herein can also be isolated by using a variety of techniques known to those of ordinary skill in the art. By way of example, such polynucleotides can be isolated through use of the polymerase chain reaction (PCR) described in Mullis et al., Eds. 1994 The Polymerase Chain Reaction, Birkhauser, incorporated herein by reference. The polynucleotides as herein disclosed can be amplified using primers, as defined herein, derived from the polynucleotide sequences as herein disclosed.

Further methods for isolating polynucleotides as disclosed herein include use of all, or portions of, the polynucleotides having the sequence set forth herein as hybridization probes. The technique of hybridizing labelled polynucleotide probes to polynucleotides immobilized on solid supports such as nitrocellulose filters or nylon membranes, can be used to screen the genomic or cDNA libraries. Exemplary hybridization and wash conditions are: hybridization for 20 hours at 65° C. in 5.0×SSC, 0.5% sodium dodecyl sulfate, 1×Denhardt's solution; washing (three washes of twenty minutes each at 55° C.) in 1.0×SSC, 1% (w/v) sodium dodecyl sulfate, and optionally one wash (for twenty minutes) in 0.5×SSC, 1% (w/v) sodium dodecyl sulfate, at 60° C. An optional further wash (for twenty minutes) can be conducted under conditions of 0.1×SSC, 1% (w/v) sodium dodecyl sulfate, at 60° C.

The polynucleotide fragments as disclosed herein may be produced by techniques well-known in the art such as restriction endonuclease digestion, oligonucleotide synthesis and PCR amplification.

A partial polynucleotide sequence may be used, in methods well-known in the art to identify the corresponding full-length polynucleotide sequence. Such methods include PCR-based methods, 5′RACE (Frohman MA, 1993, Methods Enzymol. 218: 340-56) and hybridization-based method, computer/database-based methods. Further, by way of example, inverse PCR permits acquisition of unknown sequences, flanking the polynucleotide sequences disclosed herein, starting with primers based on a known region (Triglia et al., 1998, Nucleic Acids Res 16, 8186, incorporated herein by reference). The method uses several restriction enzymes to generate a suitable fragment in the known region of a gene. The fragment is then circularized by intramolecular ligation and used as a PCR template. Divergent primers are designed from the known region. In order to physically assemble full-length clones, standard molecular biology approaches can be utilized (Sambrook et al., Molecular Cloning: A Laboratory Manual, 2nd Ed. Cold Spring Harbor Press, 1987).

It may be beneficial, when producing a transgenic plant from a particular species, to transform such a plant with a sequence or sequences derived from that species. The benefit may be to alleviate public concerns regarding cross-species transformation in generating transgenic organisms. Additionally when down-regulation of a gene is the desired result, it may be necessary to utilise a sequence identical (or at least highly similar) to that in the plant, for which reduced expression is desired. For these reasons among others, it is desirable to be able to identify and isolate orthologues of a particular gene in several different plant species.

Variants (including orthologues) may be identified by the methods described herein.

Methods for Isolating or Producing Polypeptides

The polypeptides as disclosed herein, including variant polypeptides, may be prepared using peptide synthesis methods well known in the art such as direct peptide synthesis using solid phase techniques (e.g. Stewart et al., 1969, in Solid-Phase Peptide Synthesis, WH Freeman Co, San Francisco Calif.), or automated synthesis, for example using an Applied Biosystems 431A Peptide Synthesizer (Foster City, Calif.). Mutated forms of the polypeptides may also be produced during such syntheses.

The polypeptides and variant polypeptides as disclosed herein may also be purified from natural sources using a variety of techniques that are well known in the art (e.g. Deutscher, 1990, Ed, Methods in Enzymology, Vol. 182, Guide to Protein Purification,).

Alternatively the polypeptides and variant polypeptides as disclosed herein may be expressed recombinantly in suitable host cells as disclosed herein and separated from the cells as discussed below.

Constructs, Vectors and Components Thereof

According to one embodiment, the polynucleotides useful in the methods according to some embodiments of the invention may be provided in a nucleic acid construct useful in transforming a plant or host cell. Suitable plant and host cells are described herein.

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a synthetic or recombinant polynucleotide. Once inside the host cell the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary regulatory elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
C) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

Because many microorganisms are capable of expressing multiple gene products from a polycistronic mRNA, multiple polypeptides can be expressed under the control of a single regulatory region for those microorganisms, if desired.

“Operably-linked” means that the sequenced to be expressed is placed under the control of regulatory elements that include promoters, tissue-specific regulatory elements, temporal regulatory elements, enhancers, repressors and terminators. Typically, the translation initiation site of the translational reading frame of the coding sequence is positioned between one and about fifty nucleotides downstream of the regulatory region for a monocistronic gene.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site, or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

The term “noncoding region” includes to untranslated sequences that are upstream of the translational start site and downstream of the translational stop site. These sequences are also referred to respectively as the 5′ UTR and the 3′ UTR. These sequences may include elements required for transcription initiation and termination and for regulation of translation efficiency. The term “noncoding” also includes intronic sequences within genomic clones.

Terminators are sequences, which terminate transcription, and are found in the 3′ untranslated ends of genes downstream of the translated sequence. Terminators are important determinants of mRNA stability and in some cases have been found to have spatial regulatory functions.

The term “promoter” refers to nontranscribed cis-regulatory elements upstream of the coding region that regulate gene transcription. Promoters comprise cis-initiator elements which specify the transcription initiation site and conserved boxes such as the TATA box, and motifs that are bound by transcription factors.

A “transgene” is a polynucleotide that is taken from one organism and introduced into a different organism by transformation. The transgene may be derived from the same species or from a different species as the species of the organism into which the transgene is introduced.

An “inverted repeat” is a sequence that is repeated, where the second half of the repeat is in the complementary strand, e.g.,

(5′)GATCTA . . . TAGATC(3′
(3′CTAGAT . . . ATCTAG(5′)

Read-through transcription will produce a transcript that undergoes complementary base-pairing to form a hairpin structure provided that there is a 3-5 bp spacer between the repeated regions.

Methods for Producing Constructs and Vectors

The genetic constructs as disclosed herein comprise one or more polynucleotide sequences as disclosed herein and/or polynucleotides encoding polypeptides as disclosed herein, and may be useful for transforming, for example, bacterial, fungal, insect, mammalian or plant organisms. The genetic constructs disclosed herein are intended to include expression constructs as herein defined.

Methods for producing and using genetic constructs and vectors are well known in the art and are described generally in Sambrook et al., Molecular Cloning: A Laboratory Manual, 2nd Ed. Cold Spring Harbor Press, 1987; Ausubel et al., Current Protocols in Molecular Biology, Greene Publishing, 1987).

Host Cells

In other embodiments, there is provided a host cell which comprises a genetic construct or vector as disclosed herein. In preferred embodiments, the host cell is genetically modified to i) express a polynucleotide encoding a polypeptide with the amino acid sequence of any one of SEQ ID NO: 1 to 9, or a variant of the polypeptide, or ii) express a polynucleotide comprising a nucleotide sequence selected from any one of the sequences SEQ ID NO: 10 to 18, or a variant thereof.

Host cells comprising genetic constructs, such as expression constructs, as disclosed herein are useful in methods well known in the art (e.g. Sambrook et al., Molecular Cloning: A Laboratory Manual, 2nd Ed. Cold Spring Harbor Press, 1987; Ausubel et al., Current Protocols in Molecular Biology, Greene Publishing, 1987) for recombinant production of polypeptides disclosed herein. Such methods may involve the culture of host cells in an appropriate medium in conditions suitable for or conducive to expression of a polynucleotide or polypeptide disclosed herein. The expressed recombinant polypeptide, which may optionally be secreted into the culture, may then be separated from the medium, host cells or culture medium by methods well known in the art (e.g. Deutscher, Ed, 1990, Methods in Enzymology, Vol 182, Guide to Protein Purification).

Thus, according to some embodiments, the host cells as disclosed herein are useful in the methods for producing trilobatin according to some embodiments of the invention. The host cells as disclosed herein or used according to the methods as disclosed herein preferably are, or serve as, a production strain for the biotechnological production of trilobatin as disclosed herein.

A species and strain selected for use as a trilobatin production strain is first analysed to determine which production genes are endogenous to the strain and which genes are not present. Genes for which an endogenous counterpart is not present in the strain are advantageously assembled in one or more expression constructs, which are then transformed into the strain in order to supply the missing function(s).

Exemplary prokaryotic and eukaryotic species are described in more detail below. However, it will be appreciated that other species can be suitable. For example, suitable species can be in a genus such as Agaricus, Aspergillus, Bacillus, Candida, Corynebacterium, Eremothecium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces or Yarrowia. Exemplary species from such genera include Lentinus tigrinus, Laetiporus sulphureus, Phanerochaete chrysosporium, Pichia pastoris, Pichia methanolica, Cyberlindnera jadinii, Physcomitrella patens, Rhodoturula glutinis 32, Rhodoturula mucilaginosa, Phaffia rhodozyma UBV-AX, Xanthophyllomyces dendrorhous, Fusarium fujikuroi/Gibberella fujikuroi, Candida utilis, Candida glabrata, Candida albicans, and Yarrowia lipolytica.

In some embodiments, a microorganism can be a prokaryote such as Escherichia coli, Saccharomyces cerevisiae, Rhodobacter sphaeroides, Rhodobacter capsulatus, or Rhodotorula toruloides.

In some embodiments, a microorganism can be an Ascomycete such as Gibberella fujikuroi, Kluyveromyces lactis, Schizosaccharomyces pombe, Aspergillus niger, Yarrowia lipolytica, Ashbya gossypii, or Saccharomyces cerevisiae.

In some embodiments, a microorganism can be an algal or cyanobacterial cell such as Blakeslea trispora, Dunaliella salina, Haematococcus pluvialis, Chlorella sp., Undaria pinnatifida, Sargassum, Laminaria japonica, Scenedesmus almeriensis species.

Saccharomyces spp.

Saccharomyces is a widely used chassis organism in synthetic biology, and can be used as the recombinant microorganism platform. For example, there are libraries of mutants, plasmids, detailed computer models of metabolism and other information available for S. cerevisiae, allowing for rational design of various modules to enhance product yield. Methods are known for making recombinant microorganisms.

Aspergillus spp.

Aspergillus species such as A. oryzae, A. niger and A. sojae are widely used microorganisms in food production and can also be used as the recombinant microorganism platform. Nucleotide sequences are available for genomes of A. nidulans, A. fumigatus, A. oryzae, A, clavatus, A. flavus, A. niger, and A. terreus, allowing rational design and modification of endogenous pathways to enhance flux and increase product yield. Metabolic models have been developed for Aspergillus. Generally, A. niger is cultured for the industrial production of a number of food ingredients such as citric acid and gluconic acid, and thus species such as A. niger are generally suitable for producing trilobatin.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

Aaaricus, Gibberella, and Phanerochaete spp.

Agaricus, Gibberella, and Phanerochaete spp. can be useful because they are known to produce large amounts of isoprenoids in culture. Thus, precursors for producing large amounts of phenylpropanoids, including trilobatin, are already produced by endogenous genes.

Arxula adeninivorans (Blastobotrvs adeninivorans)

Arxula adeninivorans is a dimorphic yeast with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica.

Yarrowia lipolytica is also a dimorphic yeast, and belongs to the family Hemiascomycetes. The entire genome of Yarrowia lipolytica is known. Yarrowia is aerobic and considered to be non-pathogenic. Yarrowia is efficient in using hydrophobic substrates (e.g. alkanes, fatty acids, oils) and can grow on sugars. It has a high potential for industrial applications and is an oleaginous microorganism. Yarrowia lipolyptica can accumulate lipid content to approximately 40% of its dry cell weight and is a model organism for lipid accumulation and remobilization.

Rhodotorula sp.

Rhodotorula is a unicellular, pigmented yeast. The oleaginous red yeast, Rhodotorula giutinis, has been shown to produce lipids and carotenoids from crude glycerol. Rhodotorula toruloides strains have been shown to be an efficient fed-batch fermentation system for improved biomass and lipid productivity.

Rhodosporidium toruloides

Rhodosporidium toruloides is an oleaginous yeast and useful for engineering lipid-production pathways.

Rhodobacter spp.

Rhodobacter can be used as the recombinant microorganism platform. Similar to E. coli, there are libraries of mutants available as well as suitable plasmid vectors, allowing for rational design of various modules to enhance product yield. Isoprenoid pathways have been engineered in membraneous bacterial species of Rhodobacter for increased production of carotenoid and CoQ10. Methods similar to those described above for E. coli can be used to make recombinant Rhodobacter microorganisms.

Candida boidinii

Candida boidinii is a methylotrophic yeast. Like other methylotrophic species such as Hansenuia polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH.

Hansenuia polymorpha (Pichia angusta)

Hansenula polymorpha is another methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes.

Kluyveromyces lactis

Kluyveromyces lactis is a yeast regularly applied to producing kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale.

Pichia pastoris

Pichia pastoris is a methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans).

Physcomitrella spp.

Physcomitrella mosses, when grown in suspension culture, have characteristics similar to yeast or other fungal cultures. This genera is becoming an important type of cell for producing plant secondary metabolites, which can be difficult to produce in other types of cells.

Cultivation, Expression and Isolation

In other embodiments, there is provided a method for the biosynthesis of trilobatin comprising the steps of culturing a host cell as herein disclosed, capable of expressing a 4′-O-glycosyltransferase, in the presence of phloretin which may be supplied to, or may be present in the host cell.

Trilobatin biosynthesis typically requires the co-substrates phloretin and UDP-glucose. Thus, according to one embodiment, the host cell comprises phloretin and UDP-glucose. In another embodiment, UDP-glucose and/or phloretin may be supplied to the host cell. The phloretin and UDP-glucose, each separately or combined, may be endogenous to the cell or added exogenously. Additionally, in order to produce, or upregulate production of, trilobatin, the substrates (e.g. phloretin and/or UDP-glucose) may be added exogenously to cells comprising endogenous levels of these substrates. Such a step typically results in an increase of at least about 5%, 10%, 20%, 30%, 40%, 50%, 60%, 70%, 80%, 90%, 100%, or more in substrate levels (e.g. phloretin and/or UDP-glucose) as compared to a host cell not receiving the substrates exogenously.

Additionally or alternatively, in order to produce, or upregulate production of, trilobatin, the substrate (e.g. phloretin and/or UDP-glucose) levels in a cell may be provided, or upregulated, by introducing or increasing a level of a component in the phloretin and/or UDP-glucose biosynthesis pathways. Accordingly, to produce or upregulate phloretin levels, naringin dihydrochalcone, phlorizin, phloretin-4′-O-glucoside or p-dihydrocoumaroyl-CoA may be provided or upregulated. Alternatively, the chalcone synthase or naringenin-chalcone synthase (CHS) may be provided or upregulated along with the co-substrate 3 x Malonyl-CoA for production or upregulated synthesis of phloretin. Likewise, for production or upregulation of UDP-glucose, glucose-1-phosphate may be provided or upregulated. Alternatively, UDP-glucose-pyrophosphorylase may be provided or upregulated along with the co-substrate UTP for synthesis of UDP-glucose.

Exogenous addition of a substrate (e.g. phloretin and/or UDP-glucose) may be effected using any method known in the art, such as by contacting the host cell with the substrates (e.g. phloretin and/or UDP-glucose), such as in a cell culture medium.

Expression of additional enzymes in a cell can be effected using nucleic acid constructs or using genome editing as described herein (e.g. for expression of a polypeptide comprising an amino acid sequence of any one of SEQ ID NO: 1 to 9). It will be appreciated that more than one exogenous polynucleotide(s) (e.g. 2, 3, 4, 5, etc.) may be provided in a single nucleic acid construct, alternatively, two or more (e.g. 3, 4, 5, etc.) nucleic acid constructs may be introduced into a single host cell.

In preferred embodiments, a chalcone synthase (CHS), or a chalcone synthase (CHS) and a double bond reductase (DBR) may be present, or introduced into, the host cell. For example, a suitable chalcone synthase includes HaCHS (NCBI protein accession no: Q9FUB7.1) and HvCHS2 (NCBI protein accession no: Q96562.1), and is preferably HaCHS. Suitable double bond reductases include ScTSC13 (NCBI protein accession no: NP_010269.1) and KITSC13 (NCBI protein accession XP_452392.1), and is preferably ScTSC13.

Host cells disclosed herein can be used in methods to produce trilobatin as disclosed herein, and can be cultivated using conventional fermentation processes, including, inter alia, chemostat, batch, fed-batch cultivations, continuous perfusion fermentation, and continuous perfusion cell culture.

For example, if the host cell is a microorganism (e.g. E. coli), the method can include growing the microorganism in a culture medium under conditions in which the enzyme catalyzing the step of the methods of some embodiments of the invention, e.g. 4′-O-glycosyltransferase (e.g. PGT2), is expressed. The recombinant microorganism may be grown in a fed batch or continuous process. Typically, the recombinant microorganism is grown in a fermenter at a defined temperature(s) for a desired period of time. Such a determination is within the skill of a person of skill in the art.

For example, in one embodiment of a process according to the invention, the recombinant microorganism is cultured under aerobic conditions, preferably until a maximum biomass concentration is reached. In this connection the OD₆₀₀ should preferably be at least in the range from 1 to 15 or higher, preferably in the range from 5 to 300, in particular in the range from 10 to 275, preferably in the range from 15 to 250. The microorganism is then cultured preferably under anaerobic conditions, wherein the expression of the desired amino acid sequences or the desired enzymes based on the introduced genetic construct or vector is carried out, for example by means of induction by isopropyl P-D-1-thiogalactopyranoside (IPTG) and/or lactose (when using a corresponding, suitable promoter or a corresponding, suitable expression system).

Preferably, the culturing takes place at least partially or completely under anaerobic conditions.

Depending on the microorganism, the person skilled in the art can create suitable environment conditions for the purposes of cultivation and in particular can provide a suitable (cultivation) medium. The cultivation is preferably carried out in LB or TB medium. Alternatively a (more complex) medium consisting of or comprising plant raw materials, in particular citrus, grapefruit and orange plants, are used. The cultivation is carried out for example at a temperature of more than 20° C., preferably more than 25° C., in particular more than 30° C. (preferably in the range from 30 to 40° C.).

If one or more suitable inducers, for example IPTG or lactose, are used for the induction (e.g. of the lac operon), it is preferred to use the inductor with regard to the (culture) medium that contains the recombinant microorganisms in an amount of 0.001 to 1 mM, preferably of 0.005 to 0.9 mM, particularly preferably of 0.01 to 0.8 mM.

To isolate or purify the trilobatin, extractions with organic solvents can for example be carried out. These solvents are preferably selected from the following list: isobutane, 2-propanol, toluene, methyl acetate, 2-butanol, hexane, 1-propanol, light petroleum, 1,1,1,2-tetrafluoroethane, methanol, propane, 1-butanol, butane, ethyl methyl ketone, ethyl acetate, diethyl ether, ethanol, dibutyl ether, CO₂, tert. butyl methyl ether, acetone, dichloromethane and N₂O. Particularly preferred are those solvents which form a visually recognisable phase boundary with water. After this a removal of the residual water in the solvent as well as the removal of the solvent itself can be carried out, which in turn can be followed by re-dissolving the trilobatin in a (possibly different) solvent, which for example is suitable for an optionally subsequent crystallisation and drying of the product. Alternatively or in addition an adsorptive, distillative and/or chromatographic purification can be carried out.

Alternatively, drying methods can be used for the isolation or purification of the formed trilobatin, in particular vacuum belt drying, spray drying, distillation or lyophilisation of the cell-containing or cell-free fermentation solution may be used.

Methods for Producing Plant Cells and Plants Comprising Constructs and Vectors

In other embodiments there is provided a plant cell which comprises a genetic construct as disclosed herein, and a plant cell modified to alter expression of a polynucleotide or polypeptide as disclosed herein. Plants comprising such cells are also provided.

Alteration of 4′-O-glycosyltransferase activity may be altered in a plant through methods according to some embodiments of the invention. Such methods may involve the transformation of plant cells and plants, with a construct designed to alter expression of a polynucleotide or polypeptide which modulates 4′-O-glycosyltransferase activity, or trilobatin content in such plant cells and plants. Such methods also include the transformation of plant cells and plants with a combination of the construct as disclosed herein and one or more other constructs designed to alter expression of one or more polynucleotides or polypeptides which modulate 4′-O-glycosyltransferase activity and/or trilobatin content in such plant cells and plants.

Methods for transforming plant cells, plants and portions thereof with polypeptides are described in Draper et al., 1988, Plant Genetic Transformation and Gene Expression. A Laboratory Manual. Blackwell Sci. Pub. Oxford, p. 365; Potrykus and Spangenburg, 1995, Gene Transfer to Plants. Springer-Verlag, Berlin.; and Gelvin et al., 1993, Plant Molecular Biol. Manual. Kluwer Acad. Pub. Dordrecht. A review of transgenic plants, including transformation techniques, is provided in Galun and Breiman, 1997, Transgenic Plants. Imperial College Press, London.

Methods for Genetic Manipulation of Plants

A number of plant transformation strategies are available (e.g. Birch, 1997, Ann Rev Plant Phys Plant Mol Biol, 48, 297, Hellens R P, et al (2000) Plant Mol Biol 42: 819-32, Hellens R et al Plant Meth 1: 13). For example, strategies may be designed to increase expression of a polynucleotide/polypeptide in a plant cell, organ and/or at a particular developmental stage where/when it is normally expressed or to ectopically express a polynucleotide/polypeptide in a cell, tissue, organ and/or at a particular developmental stage which/when it is not normally expressed. The expressed polynucleotide/polypeptide may be derived from the plant species to be transformed or may be derived from a different plant species.

Transformation strategies may be designed to reduce expression of a polynucleotide/polypeptide in a plant cell, tissue, organ or at a particular developmental stage which/when it is normally expressed. Such strategies are known as gene silencing strategies.

Genetic constructs for expression of genes in transgenic plants typically include promoters for driving the expression of one or more cloned polynucleotide, terminators and selectable marker sequences to detect presence of the genetic construct in the transformed plant.

The promoters suitable for use in the constructs as described herein are functional in a cell, tissue or organ of a monocot or dicot plant and include cell-, tissue- and organ-specific promoters, cell cycle specific promoters, temporal promoters, inducible promoters, constitutive promoters that are active in most plant tissues, and recombinant promoters. Choice of promoter will depend upon the temporal and spatial expression of the cloned polynucleotide, so desired. The promoters may be those normally associated with a transgene of interest, or promoters which are derived from genes of other plants, viruses, and plant pathogenic bacteria and fungi. Those skilled in the art will, without undue experimentation, be able to select promoters that are suitable for use in modifying and modulating plant traits using genetic constructs comprising the polynucleotide sequences as herein disclosed. Examples of constitutive plant promoters include the CaMV 35S promoter, the nopaline synthase promoter and the octopine synthase promoter, and the Ubi 1 promoter from maize. Plant promoters which are active in specific tissues, respond to internal developmental signals or external abiotic or biotic stresses are described in the scientific literature. Exemplary promoters are described, e.g., in WO 02/00894, which is herein incorporated by reference.

Exemplary terminators that are commonly used in plant transformation genetic construct include, e.g., the cauliflower mosaic virus (CaMV) 35S terminator, the Agrobacterium tumefaciens nopaline synthase or octopine synthase terminators, the Zea mays zein gene terminator, the Oryza sativa ADP-glucose pyrophosphorylase terminator and the Solanum tuberosum PI-II terminator.

Selectable markers commonly used in plant transformation include the neomycin phophotransferase II gene (NPT II) which confers kanamycin resistance, the aadA gene, which confers spectinomycin and streptomycin resistance, the phosphinothricin acetyl transferase (bar gene) for Ignite (AgrEvo) and Basta (Hoechst) resistance, and the hygromycin phosphotransferase gene (hpt) for hygromycin resistance.

Use of genetic constructs comprising reporter genes (coding sequences which express an activity that is foreign to the host, usually an enzymatic activity and/or a visible signal (e.g., luciferase, GUS, GFP)) which may be used for promoter expression analysis in plants and plant tissues are also contemplated. The reporter gene literature is reviewed in Herrera-Estrella et al., 1993, Nature 303, 209, and Schrott, 1995, In: Gene Transfer to Plants (Potrykus, T., Spangenberg. Eds) Springer Verlag. Berline, pp. 325-336.

Gene silencing strategies may be focused on the gene itself or regulatory elements which effect expression of the encoded polypeptide. “Regulatory elements” is used here in the widest possible sense and includes other genes which interact with the gene of interest.

Genetic constructs designed to decrease or silence the expression of a polynucleotide/polypeptide as herein disclosed may include an antisense copy of a polynucleotide as herein disclosed. In such constructs the polynucleotide is placed in an antisense orientation with respect to the promoter and terminator.

An “antisense” polynucleotide is obtained by inverting a polynucleotide or a segment of the polynucleotide so that the transcript produced will be complementary to the mRNA transcript of the gene, e.g.,

| | |
|---|---|
| 5′-GATCTA-3′ (coding strand) | 3′-CTAGAT-5′ |
| | (antisense strand) |
| 3′-CUAGAU-5′ mRNA | 5′-GAUCUA-3′ |
| | antisense RNA |

Genetic constructs designed for gene silencing may also include an inverted repeat. An ‘inverted repeat’ is a sequence that is repeated where the second half of the repeat is in the complementary strand, e.g.,

5′-GATCTA . . . TAGATC-3′
3′-CTAGAT . . . ATCTAG-5′

The transcript formed may undergo complementary base pairing to form a hairpin structure. Usually a spacer of at least 3-5 bp between the repeated region is required to allow hairpin formation.

Another silencing approach involves the use of a small antisense RNA targeted to the transcript equivalent to an miRNA (Llave et al., 2002, Science 297, 2053). Use of such small antisense RNA corresponding to polynucleotide as herein disclosed is expressly contemplated.

The term genetic construct as used herein also includes small antisense RNAs and other such polypeptides effecting gene silencing.

Transformation with an expression construct, as herein defined, may also result in gene silencing through a process known as sense suppression (e.g. Napoli et al., 1990, Plant Cell 2, 279; de Carvalho Niebel et al., 1995, Plant Cell, 7, 347). In some cases sense suppression may involve over-expression of the whole or a partial coding sequence but may also involve expression of non-coding region of the gene, such as an intron or a 5′ or 3′ untranslated region (UTR). Chimeric partial sense constructs can be used to coordinately silence multiple genes (Abbott et al., 2002, Plant Physiol. 128(3): 844-53; Jones et al., 1998, Planta 204: 499-505). The use of such sense suppression strategies to silence the expression of a polynucleotide as disclosed herein is also contemplated.

The polynucleotide inserts in genetic constructs designed for gene silencing may correspond to coding sequence and/or non-coding sequence, such as promoter and/or intron and/or 5′ or 3′ UTR sequence, or the corresponding gene.

Other gene silencing strategies include dominant negative approaches and the use of ribozyme constructs (McIntyre, 1996, Transgenic Res, 5, 257) Pre-transcriptional silencing may be brought about through mutation of the gene itself or its regulatory elements. Such mutations may include point mutations, frameshifts, insertions, deletions and substitutions.

The following are representative publications disclosing genetic transformation protocols that can be used to genetically transform the following plant species: Rice (Alam et al., 1999, Plant Cell Rep. 18, 572); apple (Yao et al., 1995, Plant Cell Reports 14, 407-412); maize (U.S. patent Ser. Nos. 5,177,010 and 5,981,840); wheat (Ortiz et al., 1996, Plant Cell Rep. 15, 1996, 877); tomato (U.S. patent Ser. No. 5,159,135); potato (Kumar et al., 1996 Plant J. 9, : 821); cassava (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. patent Ser. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. patent Ser. No. 5,792,935); soybean (U.S. Pat. Nos. 5,416,011; 5,569,834; 5,824,877; 5,563,04455 and 5,968,830); pineapple (U.S. patent Ser. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24(1):45-51); Prunus (Ramesh et al., 2006 Plant Cell Rep. 25(8):821-8; Song and Sink 2005 Plant Cell Rep. 2006; 25(2):117-23; Gonzalez Padilla et al., 2003 Plant Cell Rep.22(1):38-45); strawberry (Oosumi et al., 2006 Planta. 223(6):1219-30; Folta et al., 2006 Planta Apr 14; PMID: 16614818), rose (Li et al., 2003), Rubus (Graham et al., 1995 Methods Mol Biol. 1995; 44:129-33), tomato (Dan et al., 2006, Plant Cell Reports V25:432-441), apple (Yao et al., 1995, Plant Cell Rep. 14, 407-412) and Actinidia eriantha (Wang et al., 2006, Plant Cell Rep. 25, 5: 425-31). Transformation of other species is also contemplated by the invention. Suitable methods and protocols are available in the scientific literature.

In one embodiment, there is provided a method of producing a plant cell or plant with increased trilobatin content or increased 4′-O-glycosyltransferase activity, the method comprising upregulating in the plant cell or plant expression of a polypeptide with the amino acid sequence of any one of SEQ ID NO: 1 to 9, or a variant of the polypeptide.

In another embodiment, there is provided a method of producing a plant cell or plant with increased trilobatin content or increased 4′-O-glycosyltransferase activity, the method comprising upregulating in the plant cell or plant expression of a polynucleotide comprising a nucleotide sequence selected from any one of the sequences SEQ ID NO: 10 to 18, or a variant thereof.

Several methods known in the art may be employed to alter expression of a nucleotide and/or polypeptide as herein disclosed. Such methods include but are not limited to Tilling (Till et al., 2003, Methods Mol Biol, 2%, 205), and so called “Deletagene” technology (Li et al., 2001, Plant Journal 27(3), 235) Other methods may involve the use of sequence-specific nucleases that generate targeted double-stranded DNA breaks in genes of interest. Examples of such methods include: zinc finger nucleases (Curtin, et al., 2011, Sander, et al., 2011), transcription activator-like effector nucleases or “TALENs” (Cermak, et al., 2011, Mahfouz, et al., 2011, Li, et al., 2012), and LAGLIDADG homing endonucleases, also termed “meganucleases” (Tzfira, et al., 2012).

Targeted genome editing using engineered nucleases such as clustered, regularly interspaced, short palindromic repeat (CRISPR) technology, is an important new approach for generating RNA-guided nucleases, such as Cas9, with customizable specificities. Genome editing mediated by these nucleases has been used to rapidly, easily and efficiently modify endogenous genes in a wide variety of biomedically important cell types and in organisms that have traditionally been challenging to manipulate genetically. A modified version of the CRISPR-Cas9 system has been developed to recruit heterologous domains that can regulate endogenous gene expression or label specific genomic loci in living cells (Sander and Joung, 2014). The technique is applicable to fungi (Nodvig, et al., 2015).

Upregulating expression of a polypeptide in a plant, for example by genome editing, can be achieved by: (i) replacing an endogenous sequence encoding the polypeptide of interest or a regulatory sequence under the control which it is placed, and/or (ii) inserting a new gene encoding the polypeptide of interest in a targeted region of the genome, and/or (iii) introducing point mutations which result in up-regulation of the endogenous gene encoding the polypeptide of interest (e.g., by altering the regulatory sequences such as promoter, enhancers, 5′-UTR and/or 3′-UTR, or mutations in the coding sequence).

In this manner, an endogenous gene encoding a polypeptide with the amino acid sequence of any one of SEQ ID NO: 1 to 9 or a variant of the polypeptide, or comprising a nucleotide sequence selected from any one of the sequences SEQ ID NO: 10 to 18 or a variant thereof, may be upregulated, resulting in increased trilobatin content or increased 4′-O-glycosyltransferase activity.

Antibodies or fragments thereof, targeted to a particular polypeptide may also be expressed in plants to modulate the activity of that polypeptide (Jobling et al., 2003, Nat. Biotechnol., 21(1), 35). Transposon tagging approaches may also be applied. Additionally peptides interacting with a polypeptide as herein disclosed may be identified through technologies such as phage-display (Dyax Corporation). Such interacting peptides may be expressed in or applied to a plant to affect activity of a polypeptide as herein disclosed. Use of each of the above approaches in alteration of expression of a nucleotide and/or polypeptide as herein disclosed is specifically contemplated.

The terms “to alter expression of” and “altered expression” of a polynucleotide or polypeptide as herein disclosed, are intended to encompass the situation where genomic DNA corresponding to a polynucleotide as herein disclosed is modified thus leading to altered expression of a polynucleotide or polypeptide as herein disclosed. Modification of the genomic DNA may be through genetic transformation or other methods known in the art for inducing mutations. The “altered expression” can be related to an increase or decrease in the amount of messenger RNA and/or polypeptide produced and may also result in altered activity of a polypeptide due to alterations in the sequence of a polynucleotide and polypeptide produced.

Methods of Selecting Plants

Methods are also provided for selecting plants with altered 4′-O-glycosyltransferase or trilobatin content. Such methods involve testing of plants for altered for the expression of a polynucleotide or polypeptide as herein disclosed. Such methods may be applied at a young age or early developmental stage when the altered 4′-O-glycosyltransferase activity or trilobatin content may not necessarily be easily measurable.

The expression of a polynucleotide, such as a messenger RNA, is often used as an indicator of expression of a corresponding polypeptide. Exemplary methods for measuring the expression of a polynucleotide include but are not limited to Northern analysis, RT-PCR and dot-blot analysis (Sambrook et al., Molecular Cloning: A Laboratory Manual, 2nd Ed. Cold Spring Harbor Press, 1987). Polynucleotides or portions of the polynucleotides as herein disclosed are thus useful as probes or primers, as herein defined, in methods for the identification of plants with altered levels of 4′-O-glycosyltransferase or trilobatin. The polynucleotides as herein disclosed may be used as probes in hybridization experiments, or as primers in PCR based experiments, designed to identify such plants.

Alternatively antibodies may be raised against polypeptides as herein disclosed. Methods for raising and using antibodies are standard in the art (see for example: Antibodies, A Laboratory Manual, Harlow A Lane, Eds, Cold Spring Harbour Laboratory, 1998). Such antibodies may be used in methods to detect altered expression of the polypeptides disclosed herein. Such methods may include ELISA (Kemeny, 1991, A Practical Guide to ELISA, NY Pergamon Press) and Western analysis (Towbin & Gordon, 1994, J Immunol Methods, 72, 313).

These approaches for analysis of polynucleotide or polypeptide expression and the selection of plants with altered 4′-O-glycosyltransferase or altered trilobatin content are useful in conventional breeding programs designed to produce varieties with altered 4′-O-glycosyltransferase activity or trilobatin content.

Plants

The term “plant” is intended to include a whole plant, any part of a plant, propagules and progeny of a plant.

The term ‘propagule’ means any part of a plant that may be used in reproduction or propagation, either sexual or asexual, including seeds and cuttings.

A “transgenic” or transformed” plant refers to a plant which contains new genetic material as a result of genetic manipulation or transformation. The new genetic material may be derived from a plant of the same species as the resulting transgenic or transformed plant or from a different species. A transformed plant includes a plant which is either stably or transiently transformed with new genetic material.

The plants according to some embodiments of the invention may be grown and either self-ed or crossed with a different plant strain and the resulting hybrids, with the desired phenotypic characteristics, may be identified. Two or more generations may be grown to ensure that the subject phenotypic characteristics are stably maintained and inherited. Plants resulting from such standard breeding approaches also form an aspect of the present invention.

The function of a variant polynucleotide disclosed herein as encoding a 4′-O-glycosyltransferase may be assessed for example by expressing such a sequence in bacteria and testing activity of the encoded protein as described in the Example section herein.

Alteration of 4′-O-glycosyltransferase activity and/or trilobatin content may also be altered in a plant through methods according to some embodiments of the invention. Such methods may involve the transformation of plant cells and plants, with a construct as herein disclosed designed to alter expression of a polynucleotide or polypeptide which modulates 4′-O-glycosyltransferase activity and/or trilobatin content in such plant cells and plants. Such methods preferably also include the transformation of plant cells and plants with a combination of the construct as herein disclosed and one or more other constructs designed to alter expression of one or more other polynucleotides or polypeptides which modulate trilobatin content in such plant cells and plants. Preferably a combination of 4′-O-glycosyltransferase, a chalcone synthase (CHS), and a double bond reductase (DBR) is expressed in the plant cells or plants.

Plants that are particularly useful in the methods of the invention disclosed herein include all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arabidopsis spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incarnata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Populus spp., Prosopis cineraria, Pseudotsuga menziesii, Pterolobium stellatum, Pyrus spp., Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash and tea, amongst others.

In some embodiments, plants grown specifically for “biomass” may be used. For example, suitable plants include corn, switchgrass, sorghum, miscanthus, sugarcane, poplar, pine, wheat, rice, soy, cotton, barley, turf grass, tobacco, potato, bamboo, rape, sugar beet, sunflower, willow, and eucalyptus. In further embodiments, the plant is switchgrass (Panicum virgatum), giant reed (Arundo donax), reed canarygrass (Phalaris arundinacea), Miscanthusxgiganteus, Miscanthus sp., sericea lespedeza (Lespedeza cuneata), millet, ryegrass (Lolium multiflorum, Lolium sp.), timothy, Kochia (Kochia scoparia), forage soybeans, alfalfa, clover, sunn hemp, kenaf, bahiagrass, bermudagrass, dallisgrass, pangolagrass, big bluestem, indiangrass, fescue (Festuca sp.), Dactylis sp., Brachypodium distachyon, smooth bromegrass, orchardgrass, or Kentucky bluegrass amongst others.

Alternatively algae and other non-Viridiplantae can be used for the methods of some embodiments of the invention. In one embodiment, the plant is a plant of the Cucurbitaceae family, such as S. grosvenorii.

According to one embodiment, the plant is a plant of the Rosaceae family, such as but not limited to, apple tree, pear tree, quince tree, apricot tree, plum tree, cherry tree, peach tree, raspberry bush, loquat tree, strawberry plant, almond tree, and ornamental trees and shrubs (e.g. roses, meadowsweets, photinias, firethorns, rowans, and hawthorns).

A preferred pear genus is Pyrus.

Preferred pear species include: Pyrus calleryana, Pyrus caucasica, Pyrus communis, Pyrus elaeagrifolia, Pyrus hybrid cultivar, Pyrus pyrifolia, Pyrus salicifolia, Pyrus ussuriensis and Pyrus x bretschneideri.

A particularly preferred genus is Malus.

Preferred Malus species include: Malus aldenhamensis, Malus angustifolia, Malus asiatica, Malus baccata, Malus coronaria, Malus domestica, Malus doumeri, Malus florentina, Malus floribunda, Malus fusca, Malus halliana, Malus honanensis, Malus hupehensis, Malus ioensis, Malus kansuensis, Malus mandshurica, Malus micromalus, Malus niedzwetzkyana, Malus ombrophilia, Malus orientalis, Malus prattii, Malus prunifolia, Malus pumila, Malus sargentii, Malus sieboldii, Malus sieversii, Malus sylvestris, Malus toringoides, Malus transitoria, Malus trilobata, Malus tschonoskii, Malus x domestica, Malus x domestica x Malus sieversii, Malus x domestica x Pyrus communis Malus xiaojinensis, Malus yunnanensis, Malus sp., and Mespilus germanica.

A particularly preferred plant species is Malus domestica.

In a specific embodiment, the plant is a Malus domestica, Malus trilobata or Malus sieboldii.

In another embodiment, the plant is a plant of a Vitis species. Exemplary Vitis species include, but are not limited to, Vitis piasezkii maxim and Vitis saccharifera makino, In a preferred embodiment the plant is a plant from a species selected from a group comprising but not limited to the following genera: Smilax (eg Smilax glyciphy//a), Lithocarpus (eg Lithocarpus polystachyus), and Fragaria.

Methods for Extracting Trilobatin from Plants

Methods are also provided for the production of trilobatin by extraction of trilobatin from a plant according to some embodiments of the invention. Trilobatin may be extracted from plants by many different methods known to those skilled in the art.

Various method for extracting dihydrochalcones are known. For example, Sun et al. (2015) (incorporated herein by reference) extract trilobatin from Sweet Tea (Lithocarpus polystachyus Rehd) using a two-phase solvent system (n-Hexane-ethyl acetate-ethanol-water). Yields of 48.4 mg of trilobatin at 98.4% purity from 130 mg of crude Sweet Tea extract were obtained. Tanaka T. et al., Isolation of Trilobatin, a Sweet Dihydrochalcone-Glucoside from Leaves of Vitis piasezkii Maxim, and V. saccharifera Makino, Agricultural and Biological Chemistry, (1983) 47: 10, 2403-2404 (incorporated herein by reference) provide methods of isolation of trilobatin from Vitis leaves. Xiang-Dong Qin and Ji-Kai Liu Z., Naturforsch. (2003) 58c, 759-761 (incorporated herein by reference) provide methods of isolation of trilobatin from leaves of Lithocarpus pachyphyllus. Qin, X. et al., Dihydrochalcone Compounds Isolated from Crabapple Leaves Showed Anticancer Effects on Human Cancer Cell Lines. Molecules 2015, 20, 21193-21203 (incorporated herein by reference) provide methods of extracting trilobatin from the leaves of Malus crabapples using 50% ethanol/water. Furthermore, Xiao Z. et al., Extraction, identification, and antioxidant and anticancer tests of seven dihydrochalcones from Malus ‘Red Splendor’ fruit. Food Chem. 2017 Sep. 15; 231:324-331 (incorporated herein by reference) extract trilobatin and other dihydrochalcones from Malus ‘Red Splendor’ fruit by extraction in 80% ethanol, followed by extraction in petroleum ether and then ethyl acetate.

These methods may be up-scaled for larger scale trilobatin extraction using approaches well-known to those skilled in the art.

The term ‘comprising’ as used in this specification and claims means ‘consisting at least in part of’. When interpreting statements in this specification and claims which include the term ‘comprising’, other features besides the features prefaced by this term in each statement can also be present. Related terms such as ‘comprise’ and ‘comprised’ are to be interpreted in a similar manner.

As used herein the term ‘and/or’ means ‘and’ or ‘or’, or where the context allows both.

As used herein the term ‘(s)’ following a noun means the plural and/or singular form of that noun.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

### Examples

The invention will now be illustrated with reference to the following non-limiting examples.

It is not the intention to limit the scope of the invention to the abovementioned examples only. As would be appreciated by a skilled person in the art, many variations are possible without departing from the scope of the invention.

### 1. Example 1—Identification of 4′-O-Glycosyltransferase Genes in Apple

1.1 Materials and Methods

1.1.1 Plant material

Trilobatin production was mapped in an F1 seedling population between ‘Royal Gala’ and Y3 grown in a greenhouse at the Mt Albert Research Centre of Plant & Food Research (PFR), Auckland, New Zealand. Y3 is derived from the crabapple hydrid ‘Aotea’ x M. x domestica ‘M9’. ‘Aotea’ is derived from an open cross of M. sieboldii (which produces sieboldin). M. trilobata and ‘Aotea’ were grown at the PFR research orchard in Havelock North, New Zealand. M. micromalus ‘Makino’ and the F1 population for differential gene expression analysis between the crabapple hybrid ‘Radiant’ and M. domestica ‘Fuji’ were grown at the Luochuan Apple Experimental Station, Northwest A&F University, Shaanxi, China. All other material was grown in an experimental orchard at Northwest A&F University, Yangling, Shaanxi, China. All trees were grown on their own roots and managed using standard horticultural growth practices and management for disease and pest control.

1.1.2 Chemicals

Trilobatin was purified from Malus ‘Red Splendor’ (Xiao et al., 2017). Sieboldin, 3-OH phloretin and quercetin glycosides were purchased from PlantMetaChem (www. PlantMetaChem.com) and cyanidin from Extrasynthese (www.extrasynthese.com). All other chemicals, including phloridzin and phloretin, were obtained from Sigma Aldrich (sigmaaIdrich.com).

1.1.3 Mapping the Trilobatin Locus

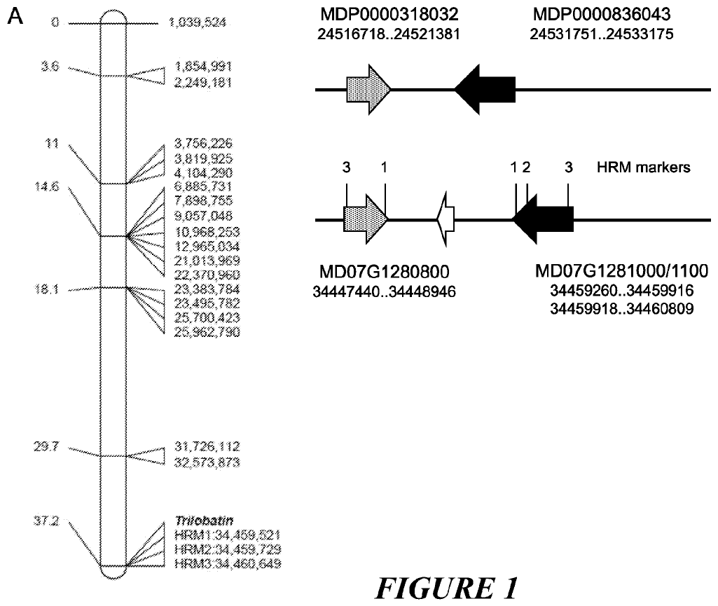

Leaf tissue from seedlings in the ‘Royal Gala’ x Y3 population were harvested and weighed before snap-freezing in liquid nitrogen. Phenolics were extracted from 100-250 mg of leaf tissue as described in Dare et al., (2017) and polyphenols quantified by Dionex-HPLC on an Ultimate 3000 system (Dionex, Sunnyvale, Calif., USA) equipped with a diode array detector at 280 nm as described in Andre et al., (2012). Seedling DNA was extracted using the DNAeasy Plant Mini Kit (Qiagen) and genotypes determined using the IRSC 8K SNP array (Chagné et al., 2012). The SNP array data was analyzed using the Genotyping Module of the GenomeStudio Data Analysis Software (Illumina). The genetic map was constructed using JoinMap version 4.0 (van Ooijen et al., 2006) and the position of the Trilobatin locus on LG7 of Y3 identified. The position of PGT2 was then defined using HRM primers designed within the PGT2 candidate genes (FIG. 1, Table 1) and PCR conditions as in Chagné et al., (2012).

TABLE 1
HRM analysis
Primer sequences for HRM anaylsis.

| | | | Co-ordinates in | Co-ordinates in |
|---|---|---|---|---|
| Marker | Name | Primers (5′→3′) | MD07G1280800 | MD07G1281000/1000 |
| HRM1 | Chr07: 34,459,521_F | ACCACTCGATTGAATCTCTG | 34,448,637-34,448,656 | 34,459,492-34,459,511 |
| | Chr07: 34,459,521_R | GTGGGTTTGTGAGTCATTG | 34,448,548-34,448,566 | 34,459,582-34,459,600 |
| HRM2 | Chr07: 34,459,729_F | AATTCCGGATCGGACTCTTT | | 34,459,705-34,459,724 |
| | Chr07: 34,459,729_R | GCGATTGGGTTGGAGAATAG | | 34,459,789-34,459,808 |
| HRM3 | Chr07: 34,460,649_F | GGATGTGGATGCACAGAGAA | 34,447,556-34,447,575 | 34,460,655-34,460,679 |
| | Chr07: 34,460,649_R | GGCGACAGCTATAGTTTTATATC | 34,447,466-34,447,490 | 34,460,570-34,460,589 |
| | | CA | | |
| HRM4 | Chr07: 34,459,983_F | GTAGACCGGTGGAGTTGG | | 34,459,956-34,459,973 |
| | Chr07: 34,459,983_R | AGCAATATGGGACGGTCT | | 34,459,993-34,460,010 |

1.1.4 Activity-Directed Protein Purification

The following protocol was used for activity-directed purification of 4′-oGT activity from the crabapple hybrid ‘Adams’ and 2′-oGT activity from M. micromalus ‘Makino’. Flower petals (50 g) were ground into fine powder with an All grinder from IKA® Works (VWR, Radnor, Pa., USA) in liquid nitrogen. The frozen powder was homogenized using a XHF-D high speed dispersator (Ningbo Scientz Biotechnology, Ningbo, China), after adding 40 ml extraction buffer (100 mM Tris-HCl, pH 7.0, 14 mM β-mercaptoethanol, 5 mM DTT, 10% glycerol, 2 mM EDTA disodium salt, and 0.5% Triton X-100) and 0.05 g·ml⁻¹ polyvinylpolypyrrolidone.

The homogenate was centrifuged at 12 000 g for 20 min, and the supernatant was collected as protein crude extract. Proteins in the supernatant were precipitated by ammonium sulfate at 30%-70% saturation. The collected pellet was dissolved in extraction buffer and desalted using PD-10 desalting columns (GE Healthcare) with buffer A (20 mM Tris-HCl, pH 8.0, 2 mM DTT). Protein solution was loaded onto a XK16/20 column packed with 10 ml Q-sepharose High Performance (GE Healthcare) which was previously equilibrated with buffer A using an AKTA Prime Plus protein chromatography system (GE Healthcare). Proteins were eluted with a liner gradient of 0%-100% of buffer B (buffer A +1 M NaCl) in ten column volumes at a flow rate of 1 ml·min⁻¹. Each fraction of 2 ml was collected and assayed for GT activity using HPLC. Fractions with high GT activity were pooled and the solvent was exchanged to buffer C (20 mM phosphate buffer, pH 7.0, 2 mM DTT, 1 M ammonium sulfate) with an ultrafiltration centrifuge tube (Vivaspin Turbo15, www.sartorius.com). This fraction was then loaded onto another XK16/20 column packed with 10 ml Phenyl Sepharose High Performance (GE Healthcare) equilibrated with buffer C. The protein was eluted with a linear gradient of 100%-0% buffer C with 10x column volumes at a flow rate of 1 ml·min⁻¹. Each fraction of 2 ml was collected and the active fractions were pooled and desalted using an ultrafiltration centrifuge tube in buffer A. The proteins were further purified on a XK16/70 column packed with 120 ml Superdex 75 preparative grade (GE Healthcare), equilibrated, and eluted with buffer A at a flow rate of 0.8 ml·min⁻¹. Each fraction of 1 ml was collected and assayed for GT activity. Each active fraction was concentrated separately using an ultrafiltration centrifuge tube, and then used for SDS-PAGE analysis. All protein purification steps were performed at 0-4° C. Column temperatures were controlled using a THD-06H circulating water bath (Tianheng Instruments, Ningbo, China).

Purified protein fractions were separated on 12% SDS-PAGE gels and visualized by Coomassie Blue R-250 staining. Target bands were cut and digested in gel with trypsin according to Gao et al., (2017). The peptide mixture was then loaded onto a reverse phase trap column (Thermo Scientific Acclaim PepMap 100, 100 μm×2 cm, nanoViper C18) connected to the C18-reversed phase analytical column (Thermo Scientific Easy Column, 10 cm long, 75 μm inner diameter, 3 μm resin) in buffer A (0.1% formic acid) and separated with a linear gradient of buffer B (84% acetonitrile and 0.1% formic acid) at a flow rate of 300 nL·min⁻¹ controlled by IntelliFlow technology. LC-MS/MS analysis was performed on a Q Exactive mass spectrometer (Thermo Scientific) that was coupled to Easy nLC (Proxeon Biosystems, now Thermo Scientific) for 60 min, and the mass spectrometer was operated in positive ion mode. MS/MS spectra were searched using MaxQuant software version 1.5.3.17 (Max Planck Institute of Biochemistry, Martinsried, Germany) against NCBI and the M. x domestica database (Malus_x_domestica.v1.0-primary.protein.fa.gz) available at www.rosaceae.orq.

1.1.5 Differential Gene Expression Analysis

The F1 population developed from a cross between the crabapple hybrid ‘Radiant’ and M. x domestica ‘Fuji’ was screened for trilobatin and phloridzin by HPLC. Eighty-one plants containing trilobatin+phloridzin (T+P) and 81 plants containing only phloridzin (P) were identified. One expanding leaf was collected from each seedling (˜20 cm tall) and three pooled replicate samples for T+P and P were prepared (each replicate containing leaves from 27 plants). Total RNA was extracted from frozen ground powder using Trizol Reagent (Life Technologies) following the manufacturer's instructions and checked for RNA integrity on an Agilent Bioanalyzer 2100. Sequencing libraries were generated from 3 μg RNA per sample using NEBNext Ultra RNA Library Prep Kit for Illumina (Thermo Fisher) following the manufacturer's recommendations and index codes were added to attribute sequences to each sample. RNA was sequenced by Novogene (Beijing, China) using the Illumina HiSeq4000 platform.

For transcriptome analysis, RNA from three biological replicates of each sample was sequenced by Novogene (Beijing, China) using the Illumina Hiseq4000 platform. Reads were aligned to the M. x domestica ‘Golden Delicious’ v1.0p assembly (https://www.rosaceae.org/species/malus/malus_x_domestica/genome_v1.0) with BOWTIE v2.2.3 and TopHat v2.0.12. Differential gene expression analysis was performed using the DEGSeq R package (1.26.0).

1.1.6 qRT-PCR Analysis

Total RNA was extracted from young leaves as described by Malnoy et al., (2001). First-strand cDNA was synthesized from 1 μg of total RNA using the PrimeScript RT Reagent Kit (Takara, Dalian, China), according to the manufacturer's instructions. qRT-PCR was performed with a Bio-Rad CFX96 system (Bio-Rad Laboratories, Hercules, Calif., USA) using the TB Green Premix Ex Taq (Takara, Dalian, China). MdActin was used as the reference gene. The relative expression levels were calculated according to the 2 ^{ΔΔCT} method (Livak and Schmittgen, 2001). Three biological replicates each with three technical repeats were used for qRT-PCR analysis. Gene-specific primers are listed in Table 2.

TABLE 2
qRT-PCR
Primer sequences for qRT-PCR. All primer efficiencies were >1.85.

| | | | | | Product size |
|---|---|---|---|---|---|
| Target | Name | Primer (5′→3′) | Name | Primer (5′→3′) | (bp) |
| MdActin | MdActinF | TGACCGAATGAGCAAGGAAATTACT | MdActinR | TACTCAGCTTTGGCAATCCACATC | 156 |
| PGT1 | PGT1F | AGCAAACCAAAACCACCGAG | PGTIR | TTGGGAAGATGTGAGCAGAAATA | 163 |
| PGT2 | Q88A1F | TCGGGGTCTCGTGGTCAAG | Q88A1R | ACTCGTTCATCGGCATAGC | 212 |
| PGT3 | Q88A1- | CCCCACCATTCACAACATCAC | Q88A1-304R | TAGGAAATGTTCGTACGCCTT | 142 |
| | 304F | | | | |
| MdCHS | CHSF | CAGCGTTGATTTATCTATCTGCTTC | CHSR | TGCACCAAGTTAACCCCATGACG | 133 |
| | | TGC | | | |

1.2 Results

1.2.1 Mapping Levels of Trilobatin in a Segregating Population

Trilobatin levels were mapped in a segregating population developed from a cross between domesticated and wild apples (‘Royal Gala’ x Y3). The female parent ‘Royal Gala’ (M. x domestica) produced only phloridzin, whilst the male parent Y3 (derived from M. sieboldii) produced both trilobatin and phloridzin. Of the fifty-one plants phenotyped, 30 contained trilobatin and phloridzin and 21 phloridzin alone. The segregation ratio (1:0.7; X²=1.58) suggested trilobatin content was segregating as a qualitative trait controlled by a single gene. Data obtained by screening leaf DNA with the International RosBREED SNP Consortium (IRSC) 8K SNP array (Chagné et al., 2012) were analyzed using JoinMap version 4.0 (van Ooijen et al., 2006). A single locus for control of trilobatin biosynthesis (Trilobatin) was identified on the lower arm of Linkage Group (LG)7 distal to a single nucleotide polymorphism (SNP) marker located at position 32,527,873 bp (FIG. 1A) on the ‘Golden Delicious’ doubled-haploid genome assembly (GDDH13 v1.1) (Daccord et al., 2017). The locus was further defined using high resolution melting (HRM) SNP markers developed from two candidate PGT genes at 34,460,00-34,461,000 bp FIG. 1A, B) close to the base of LG7 (total length of LG7 is 36,691,129 in GDDH13 v1.1). The position mapped on LG7 was consistent with one of the three independently segregating loci for dihydrochalcone content reported recently in Malus, using linkage and association analysis (Gutierrez et al., 2018a).

1.2.2 Candidate Glycosyltransferases Identified by Activity-Directed Protein Purification

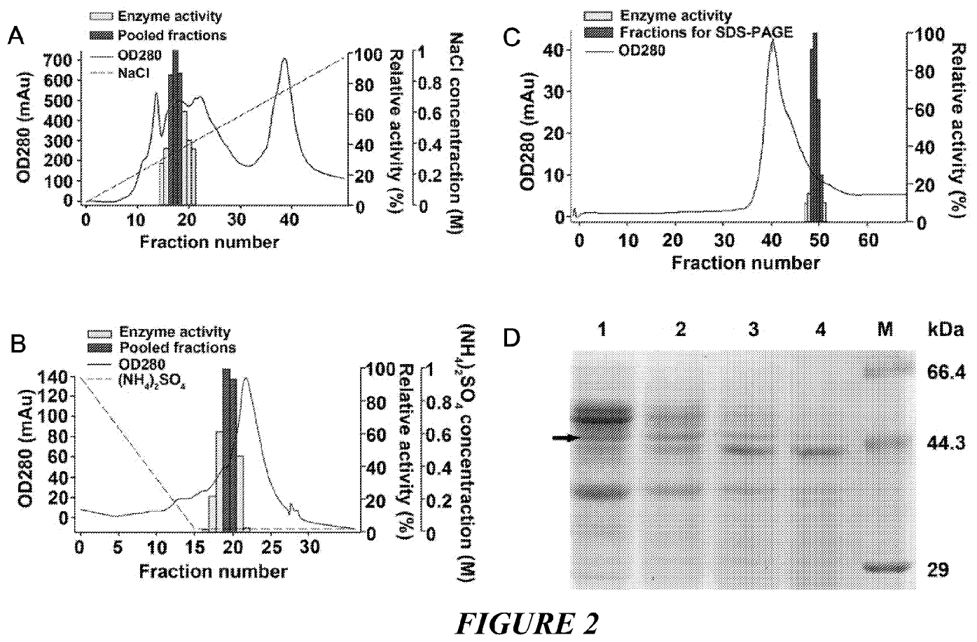

Tissues high in 4′-oGT activity required for trilobatin production, but containing very low 2′-oGT activity for phloridzin synthesis, were used to identify candidate 4′-oGTs by activity-directed protein purification. Flower petals of the crabapple hybrid ‘Adams’ were identified as a suitable experimental material as they have low levels of Rubisco compared with leaves, but higher 4′-oGT activity compared with fruit. Purification involved sequential chromatographic steps (Q-sepharose, phenyl sepharose and Superdex 75; FIG. 2A-C), after which fractions with high 4′-oGT activity were pooled and used for further purification. In the final step, after size exclusion chromatography, four protein fractions with different 4′-oGT enzyme activities were analyzed by SDS-PAGE. The abundance of a single band between 44-66 kDa (the size expected of a typical UGT) changed in a similar pattern (FIG. 2D) to that of the 4′-oGT activities (FIG. 2C). This band was subjected to LC-MS/MS analysis and peptides corresponding to 50 proteins were identified in the M. x domestica ‘Golden Delicious’ v1.0 genome assembly (Velasco et al., 2010). The five most abundant proteins are listed in

Table. Gene model descriptions in Column 2 were obtained by BLASTn searches of NCBI. IBAQ=sum of all peptide intensities divided by the number of observable peptides of a protein. The analysis was performed twice and the most abundant proteins found in both analyses (R1, R2) are given in Columns 3 and 4. Peptides corresponding to gene models MDP0000836043/MDP0000318032, encoding predicted UDP-glycosyltransferase 88A1-like proteins, were observed at highest abundance (26% of total peptides).

TABLE 3
Abundant proteins identified by LC-MS/MS analysis of
bands isolated after activity-directed purification
of 4′-oGT activity from flowers of the crabapple
hybrid ‘Adams’ (containing trilobatin and not phloridzin).

| Protein | Description | iBAQ R1 | iBAQ R2 |
|---|---|---|---|
| MDP0000836043/ | M. × domestica | 3786800000 | 3146000000 |
| MDP0000318032 | UDP-glycosyl- | | |
| | transferase | | |
| | 88A1-Iike | | |
| MDP0000155691 | M. × domestica | 96169000 | 33639000 |
| | pentatricopeptide | | |
| | repeat-containing | | |
| | protein At4g14190, | | |
| | chloroplastic | | |
| MDP0000267350 | M. × domestica | 85148000 | 273050000 |
| | monodehydroascorbate | | |
| | reductase-like | | |
| MDP0000705244 | M. × domestica | 75047000 | 628300 |
| | UDP-glycosyl- | | |
| | transferase | | |
| | 76B1-Iike | | |
| MDP0000234480 | M. × domestica | 31272000 | 246460000 |
| | transaldolase-like | | |

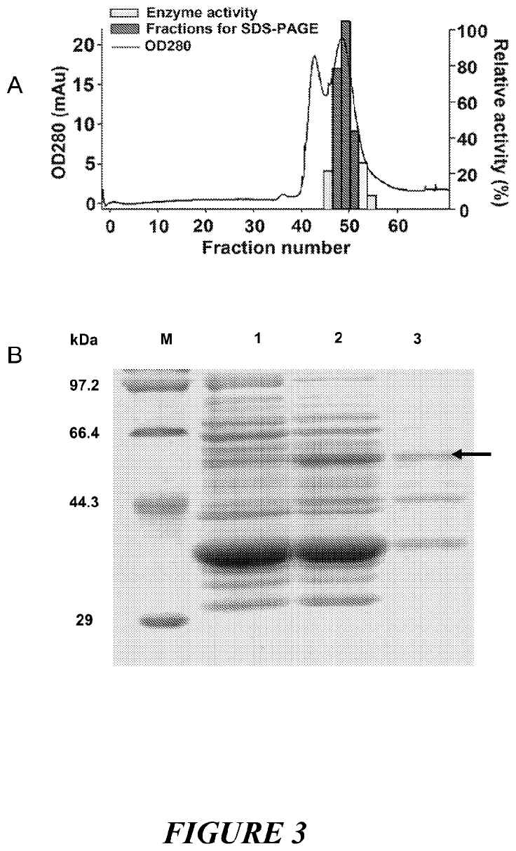

M. micromalus ‘Makino’ flower petals that show high 2′-oGT activity for phloridzin biosynthesis, but no 4′-oGT activity, were used for activity-directed purification of candidate 2′-oGTs using the method described above. After size exclusion chromatography, the abundance of a single band between 44-66 kDa changed in a pattern corresponding with 2′-oGT activity (FIG. 3). After LC-MS/MS analysis of this band, peptides corresponding to MDP0000219282/MDP0000052862 were observed at highest abundance (88% of the total peptides). MDP0000219282 encodes MdPGT1 (UDP-glycosyltransferase 88F1), a phloretin-specific 2′-oGT previously described by Jugdé et al., (2008).

1.2.3 Candidate Glycosyltransferases Identified by Differential Gene Expression Analysis

A second approach using differential gene expression (DGE) analysis as described in section 1.1.5 was used to identify candidate 4′-oGTs in tissues high in trilobatin but low in phloridzin. A cross was produced between the ornamental crabapple hybrid ‘Radiant’ (containing both trilobatin and phloridzin), and M. x domestica ‘Fuji’ (containing only phloridzin). The F1 progeny were separated into two phenotypes with or without trilobatin for RNA extraction and transcriptome analysis. Expression levels of 109 genes were up-regulated at least log 2-fold change >4 in progeny producing trilobatin. The five genes showing the greatest log-fold change are shown in

Table 4. Gene model descriptions in Column 2 were obtained by BLASTn searches of NCBI. The expression level of the predicted UDP-glycosyltransferase 88A1-like protein MDP0000836043 exhibited the largest differential expression, and was up-regulated over log 2-fold change >7 in plants with trilobatin compared to those without.

TABLE 4
The five most differentially expressed genes identified after
transcriptome analysis of pooled leaf samples of an F1 population
between the crabapple hybrid ‘Radiant’ (containing both
trilobatin and phloridzin) and M. × domestica ‘Fuji’
(containing only phloridzin).

| | | log2-fold |
|---|---|---|
| Gene | Description | change |
| MDP0000836043 | M. × domestica | 7.54 |
| | UDP-glycosyltransferase | |
| | 88A1-Iike | |
| | (LOC103410306), mRNA | |
| MDP0000204525 | M. × domestica | 6.89 |
| | cinnamoyl-CoA reductase 1 | |
| | (LOC103427062), mRNA | |
| MDP0000206483 | M. × domestica | 6.77 |
| | cytokinin hydroxylase-like | |
| | (LOC114826167), mRNA | |
| MDP0000219066 | M. × domestica | 6.68 |
| | cytochrome P450 CYP72A219-like | |
| | (LOC103427349), mRNA | |
| MDP0000737403 | M. × domestica | 6.63 |
| | probable mannitol dehydrogenase | |
| | (LOC103446373), mRNA | |

1.2.4 Genetic Mapping of Candidate Genes and Expression Analysis

Genetic mapping by HRM marker analysis of gene model MDP0000836043, the candidate UDP-glycosyltransferase 88A1-like protein identified by activity-directed protein purification and DEG analysis, demonstrates that it co-locates with the locus identified for trilobatin production on LG7 (FIG. 1A). In the ‘Golden Delicious’ v1.0p assembly (Velasco et al., 2010) MDP0000836043 is located at -24,531,751 bp and corresponds to gene models MD07G1281000/1100 (located at 34,459,260 bp) in the doubled-haploid assembly GDDH13 v1.1 (FIG. 1B). The second UDP-glycosyltransferase 88A1-like protein identified by activity-directed protein purification encodes by MDP0000318032 (MD07G1280800) is located ˜10.3 kb away in both assemblies (FIG. 1B). Four SNP variants identified in the region of these two UDP-glycosyltransferase gene models were used to develop markers for HRM analysis (HRM primer sequences in Table 1). The mapping results validated the position of the MDP0000836043 and MDP0000318032 on LG7 (FIG. 1A), and three of the markers (HRM1-3) showed precise concordance with presence/absence of trilobatin in the segregating progeny.

TABLE 5
Phenotype-to-genotype comparisons for individuals used to construct
the genetic map (FIG. 1A) and in HRM assays (FIG. 1B). The phenotype
column shows presence (+) and absence (−) of trilobatin determined
by HLPC analysis of leaf samples. The genotype columns show presence
(+) and absence (−) of bands obtained in the four HRM assays.

| HPLC | Sample | HRM1 | HRM2 | HRM3 | HRM4 |
|---|---|---|---|---|---|
| phenotype | name | 34,459,521 | 34,459,729 | 34,460,649 | 34,459,983 |
| | 4031-17* | | | | |
| − | 4031-07* | − | − | − | − |
| − | 4031-11* | − | − | − | − |
| − | 4031-12* | − | − | − | − |
| − | 4031-06* | − | − | − | + |
| − | 4031-13* | − | − | − | − |
| − | 4051-30* | − | − | − | + |
| − | 4031-02* | − | − | − | − |
| − | 4031-03* | − | − | − | + |
| + | 4051-08* | + | + | + | + |
| + | 4031-20* | + | + | + | + |
| + | 4051-14* | + | + | + | − |
| + | 4031-01* | + | + | + | − |
| + | 4031-08* | + | + | + | + |
| + | 4051-16* | + | + | + | − |
| + | 4031-09* | + | + | + | + |
| + | 4031-26* | + | + | + | − |
| + | 4051-19* | + | + | + | − |
| + | 4031-10* | + | + | + | + |
| + | 4051-26* | + | + | + | − |
| + | 4031-30* | + | + | + | + |
| + | 4031-04* | + | + | + | + |
| + | 4031-14* | + | + | + | + |
| + | 4031-32* | + | + | + | + |
| + | 4051-35* | + | + | + | − |
| + | 4031-15* | + | + | + | − |
| + | 4031-19* | + | + | + | + |
| + | 4031-16* | + | + | + | + |
| + | 4024-01 | + | + | + | − |
| + | 4024-02 | + | + | + | + |
| + | 4031-01 | + | + | + | − |
| − | 4031-21 | − | − | − | + |
| − | 4031-22 | − | − | − | − |
| + | 4031-23 | + | + | + | + |
| + | 4031-25 | + | + | + | − |
| − | 4031-27 | − | − | − | + |
| − | 4031-29 | − | − | − | − |
| + | 4031-31 | + | + | + | − |
| + | 4031-33 | + | + | + | − |
| − | 4031-36 | − | − | − | − |
| + | 4031-37 | + | + | + | + |
| − | 4031-38 | − | − | − | − |
| + | 4051-03 | + | + | + | + |
| − | 4051-06 | − | − | − | + |
| − | 4051-27 | − | − | − | − |
| − | 4051-37 | − | − | − | + |

*samples included in construction of the genetic map using 8K SNP array data.

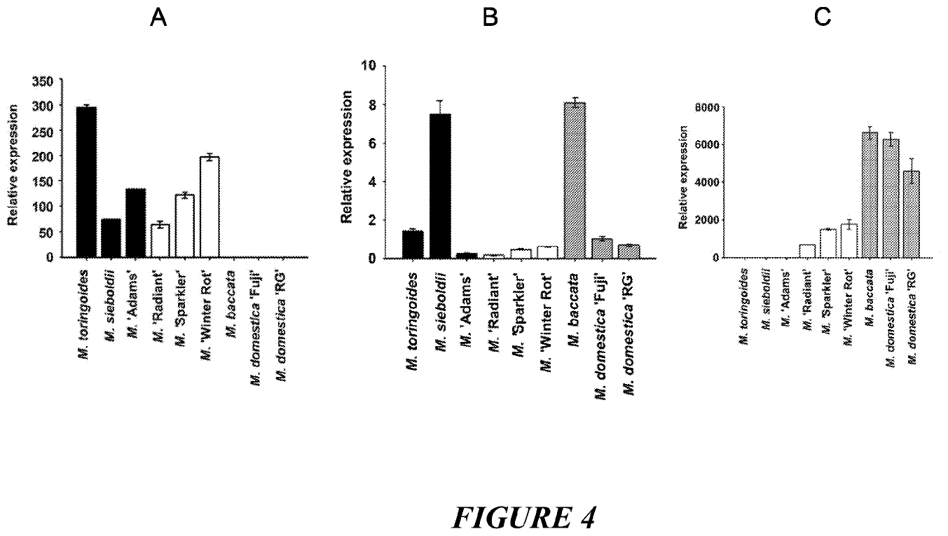

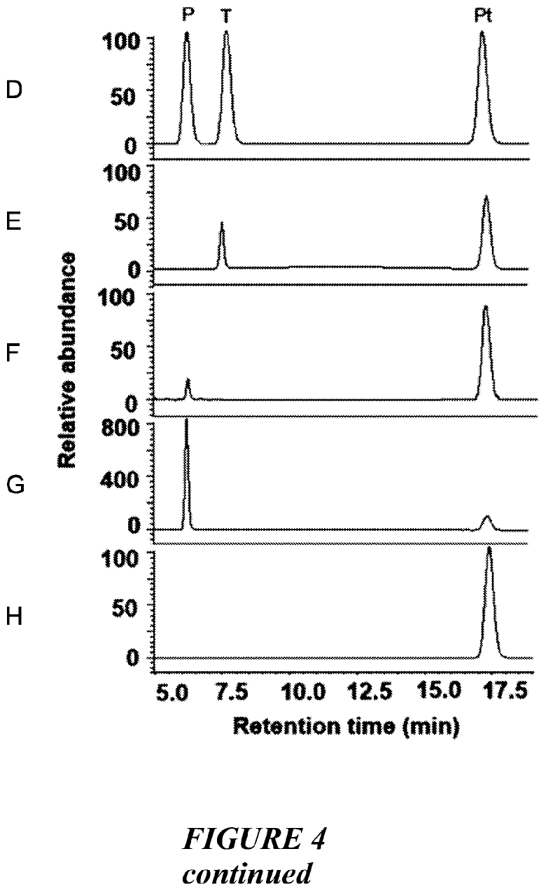

The relative expression of MDP0000836043 (hereafter termed PGT2) and MDP0000318032 (termed PGT3) were determined by qRT-PCR in the leaves of nine Malus accessions (FIG. 4); three producing predominantly trilobatin, three producing both trilobatin and phloridzin, and three producing only phloridzin. PGT2 was highly expressed in all six Malus accessions producing trilobatin, however expression was essentially absent in the three accessions that do not synthesize trilobatin (FIG. 4A). Conversely the expression of PGT1 was high in the six Malus accessions producing phloridzin (FIG. 4B). Expression of PGT3 was observed in all nine accessions, and did not correlate with the presence/absence of trilobatin or phloridzin in the samples (FIG. 4C).

1.3 Discussion

Glycosyltransferases are encoded by large gene families and identifying enzymes with specific activities based on homology is difficult. Two enzymes capable of 4′-O-glycosylation of phloretin in vitro have been reported (Gosch et al., 2012; Yahyaa et al., 2016), but these genes are expressed in tissues that produce only phloridzin. In this Example, the inventors used multiple approaches to show that phloretin glycosyltransferase 2 is responsible for production of trilobatin in apple. The genetic locus for trilobatin production co-located with the PGT2 gene and HRM markers developed to PGT2 segregated strictly with trilobatin production. In addition, molecular and biochemical analysis described in Example 2 demonstrates that PGT2 was only expressed in accessions where trilobatin (or sieboldin) was produced and that the enzyme showed 4′-oGT activity in vitro.

### 2. Example 2—Expression and Biochemical Analysis of PGT2 and PGT3 in Escherichia coli

2.1 Materials and Methods

2.1.1 Chemicals

Trilobatin was purified from Malus ‘Red Splendor’ (Xiao et al., 2017). Sieboldin, 3-OH phloretin and quercetin glycosides were purchased from PlantMetaChem (www. PlantMetaChem.com) and cyanidin from Extrasynthese (www.extrasynthese.com). All other chemicals, including phloridzin and phloretin, were obtained from Sigma Aldrich (sigmaaIdrich.com).

2.1.2 Cloning

The ORFs of PGT1-3 were amplified using primers in Table 6 and ligated into pET28a(+) (www.novagen.com) using the One Step Cloning Kit (www.vazyme.com).

TABLE 6
Cloning
Primer sequences for cloning. Restriction sites used for cloning are underlined.

| | Forward primer | | Reverse primer | |
|---|---|---|---|---|
| Name | (5′→3′) | Name | (5′→3′) | Purpose |
| 28a-88A1F | TAAGAAGGAGATATACCATGG | 28a-88A1R | GTGGTGGTGGTGGTGCTCGAG | Cloning PGT2 |
| | AGGCGACAGCTATAGTTTTA | | ACAGGTTTTGCCCCAGAATTC | (M. toringoides-2) into |
| | | | A | pET28a(+) for expression |
| | | | | in E. coli |
| 28a-88F1F | TAAGAAGGAGATATACCATGG | 28a-88F1R | GTGGTGGTGGTGGTGCTCGAG | Cloning PGT1 |
| | GAGACGTCATTGTACTGTA | | TGTTATGCTATTAACAAAGTT | (M. x domestica ‘Fuji’) |
| | | | GACCAA | into pET28a(+) |
| 28a-304F | TAAGAAGGAGATATACCATGG | 28a-304R | GTGGTGGTGGTGGTGCTCGAG | Cloning PGT3 |
| | ATGGAGGCGGCAGCTATAGTT | | CTAACCAGTTTTGCCCCACAA | (M. sieboldii-2) |
| | TTATA | | TTGAA | into pET28a(+) |

2.1.3 Protein expression in E. coli

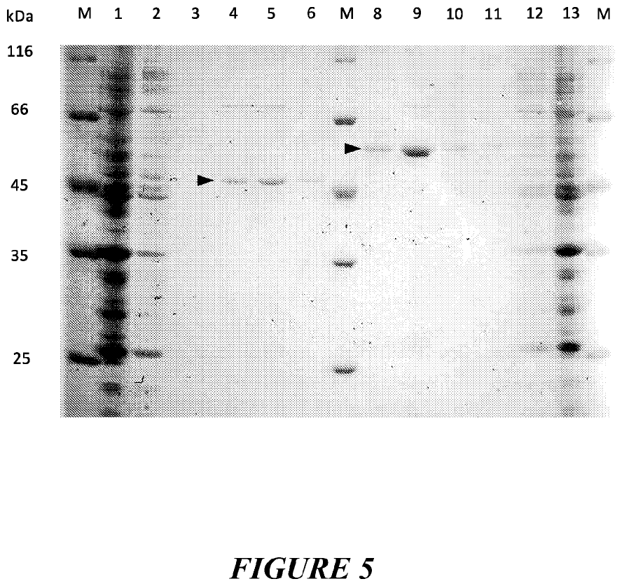

Recombinant proteins were expressed in E. coli BL21 (DE3) cells with 0.5 mM isopropyl-1 thio-β-galactopyranoside (IPTG) at 16° C. for 24 h at 80 rpm. Purification of recombinant proteins was performed using Ni-NTA agarose (Millipore). Eluted fractions were used for determining enzyme activity and for SDS-PAGE analysis (FIG. 5). Active fractions were concentrated using Vivaspin 2 concentrators (Sartorius, Germany).

2.1.4 Glycosyltransferase Activity Assays

GT activity assays were performed in 200 μL reactions containing 50 mM Tris-HCl (pH 9.0), 1 mM DTT, 0.5 mM phloretin, 0.5 mM UDP-glucose, and 30-80 ng enzyme. Reaction mixtures were incubated for 10 min at 40° C. and reactions stopped by adding 40 μL of 1 M HCl. NaOH (1 M) was used to adjust the pH to neutral for HPLC analysis of the products at 280 nm.

The activity of PGT2 and PGT1 were tested at 37° C., over the pH range 4-12, using a number of buffer systems: 0.1 M Na-citrate buffer at pH 4.0, 5.0, 6.0; 0.1 M Tris-HCl buffer at pH 7.0, 8.0, 9.0, 10.0 and 0.1 M Na₂HPO₄-NaOH at pH 11.0, 12.0; Glycine-NaOH buffer at pH 8.6, 9.0, 10.0, 10.6; and Britton-Robinson buffer at pH 6.0-11.0.

To determine the temperature optima of PGT2 and PGT1, reactions were carried out at pH 9.0 in 0.1 M Tris-HCl buffer at 15-50° C. and 15-60° C. respectively. Reactions to determine Kₘ values were performed at pH 9.0 in 0.1 M Tris-HCl buffer, 40° C. for 10 min. The Kₘ values of phloretin (E) were determined at concentrations from 4-500 μM at a fixed UDP-glucose concentration of 500 μM. The Kₘ values of UDP-glucose were determined at concentrations from 2-500 μM with a fixed phloretin concentration of 500 μM. Kₘ values were calculated by non-linear regression in Sigmaplot.

2.2 Results

2.2.1 Cloning PGT2 and PGT3

The complete open reading frame (ORF) of PGT2 was amplified from the leaves of six Malus accessions. The PGT2 ORFs from five accessions synthesizing trilobatin showed 91-94% amino acid identity to the MDP0000836043 gene model from the M. x domestica ‘Golden Delicious’ v1.0p assembly available at www.rosaceae.ora (FIG. 6). GenBank accession numbers: M. x domestica ‘Fuji’-1 (MN381003), M. toringoides-1 (MN380999), M. toringoides-2 (MN381000), M. sieboldii-1 (MN381001), crabapple hybrid ‘Adams’-1 (MN381002), crabapple hybrid ‘Aotea’-1 (MN381006), M. trilobata-1 (MN381004) and M. trilobata-2 (MN381005).

All accessions produce trilobatin except M. x domestica ‘Fuji’. A complete ORF for PGT2 obtained from the leaves of M. domestica ‘Fuji’ was identical to that obtained from M. toringoides, although PGT2 was difficult to obtain from ‘Fuji’ due to very low expression levels.

2.2.2 Expression in E. coli and activity assays

PGT2 and PGT3 from five Malus accessions and PGT1 from ‘Fuji’ were expressed in E. coli and the products formed using phloretin and UDP-glucoside as substrates were determined by HPLC. All PGT2 enzymes produced a single peak at 7.5 min that ran at the same retention time as the trilobatin standard (FIG. 4D). A representative HPLC trace for the product produced by PGT2 from M. toringoides is shown in FIG. 4E. All PGT3 enzymes (FIG. 4F) and PGT1 from ‘Fuji’ (FIG. 4G) produced a peak at 6.0 min with the same retention time as phloridzin (FIG. 4D), but no trilobatin. No phloridzin or trilobatin were produced by the empty vector control (FIG. 4H).

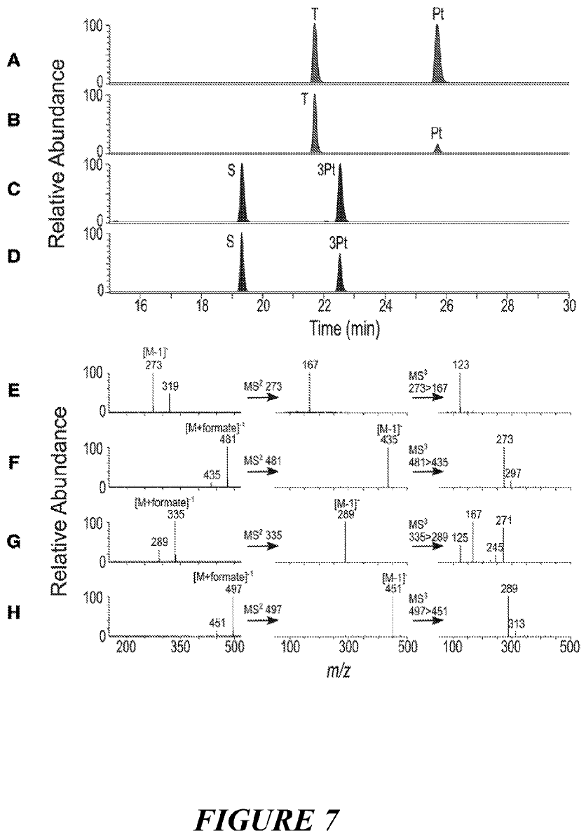

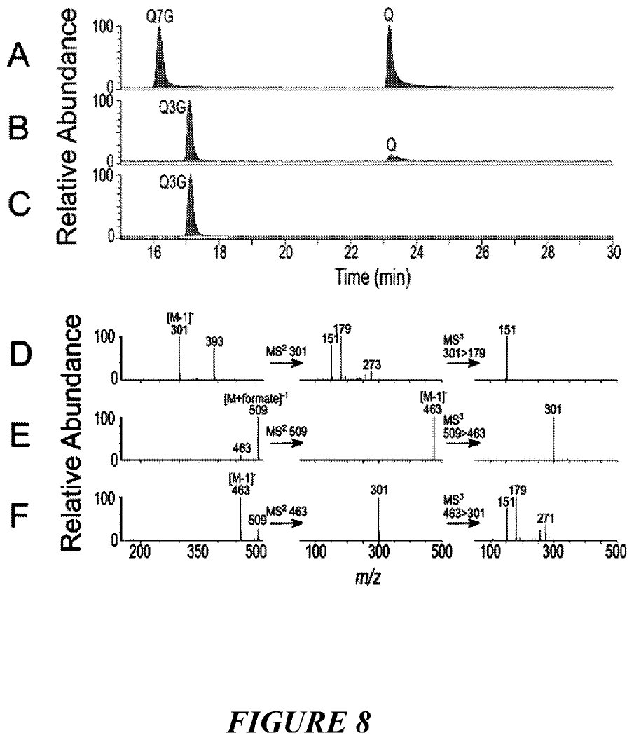

The substrate specificity of recombinant PGT2 from M. toringoides was further characterized using UDP-glucoside as the sugar donor and twelve substrates typically found in apple or with structural homology to phloretin. The products of each reaction were determined by LC-MS/MS. Phloretin was the best acceptor for PGT2 and base peak plots indicated that a single peak at 21.5 min was formed that co-eluted with the trilobatin standard (FIG. 7A, B). PGT2 also catalyzed glycosylation of 3-OH phloretin to produce sieboldin (FIG. 7C, D) with a relatively high conversion rate of ˜60% (Table 7). Quercetin-3-O-glucoside was detected as a reaction product using quercetin (FIG. 8A-F) with a lower conversion rate of 9.1% (Table 7).

TABLE 7
Substrate specificity of recombinant PGT2 cloned from
M. toringoides. The products of reactions using UDP-
glucoside as the sugar donor and the twelve substrates
shown were determined by LC-MS/MS. Conversion % is the
amount of product formed relative to the conversion
of phloretin to trilobatin which was set at 100%.

| | | Conversion |
|---|---|---|
| Substrate | Product | (%) |
| phloretin | Trilobatin | 100.0 ± 6.1 |
| 3-OH phloretin | Sieboldin | 58.7 ± 2.3 |
| quercetin | quercetin 3-O- | 9.1 ± 0.1 |
| | glucoside | |
| phloridzin | Nd | 0 |
| trilobatin | Nd | 0 |
| sieboldin | Nd | 0 |
| naringenin | Nd | 0 |
| cyanidin | Nd | 0 |
| caffeic acid | Nd | 0 |
| 4-coumaric acid | Nd | 0 |
| neohesperidin | Nd | 0 |
| chlorogenic acid | Nd | 0 |
| boiled protein | Nd | 0 |

nd = no products detected.

This result is surprising as the 4′ position of dihydrochalcones corresponds to the 7 position of quercetin, but no quercetin-7-O-glucoside was observed. No products were detected in reactions with the other substrates. Fullscan and MS/MS mass spectral data were used to further characterize the products of the PGT2 reactions. Phloretin (FIG. 7E) was detected as its pseudo-molecular ion m/z 273 [M-1]-), whereas trilobatin (FIG. 7F), 3-OH phloretin (FIG. 7G) and sieboldin (FIG. 7H) were detected predominately as the corresponding formate adducts [M+formate])-1. MS2 on the formate adducts, identified the expected pseudo-molecular ion at m/z 435 and 451 [M-1]-) for the trilobatin and sieboldin glucosides. MS3 on the m/z 435 and 451 [M-1]-) glucoside ions identified the m/z 273 and m/z 289 [M-1]-) ions of the phloretin and 3-OH phloretin aglycones respectively.

PGT2 and PGT1 enzyme activities were compared over a pH range of 4-12 and with temperatures from 15-60° C., as follows:

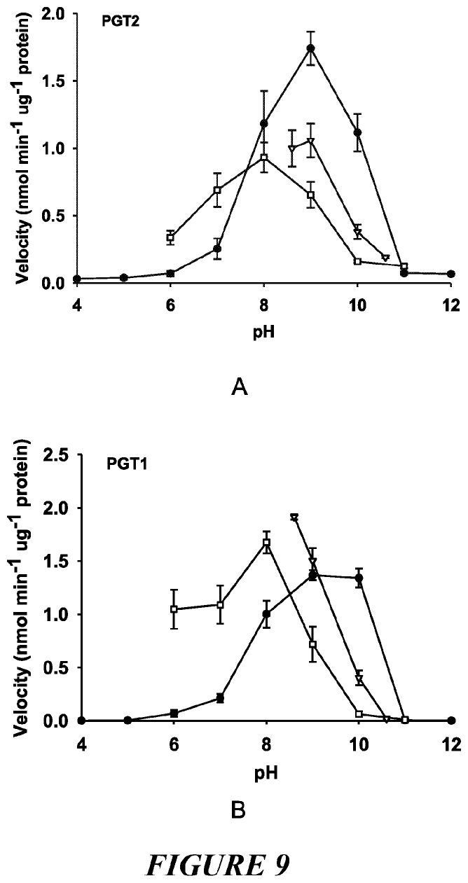

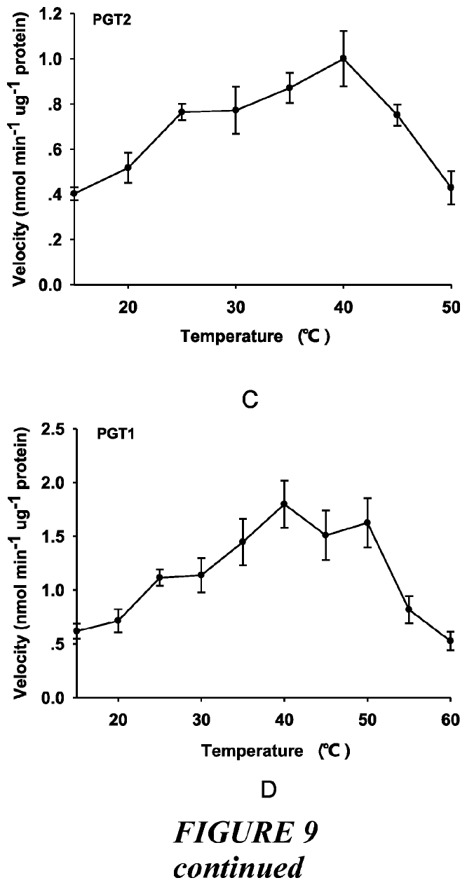

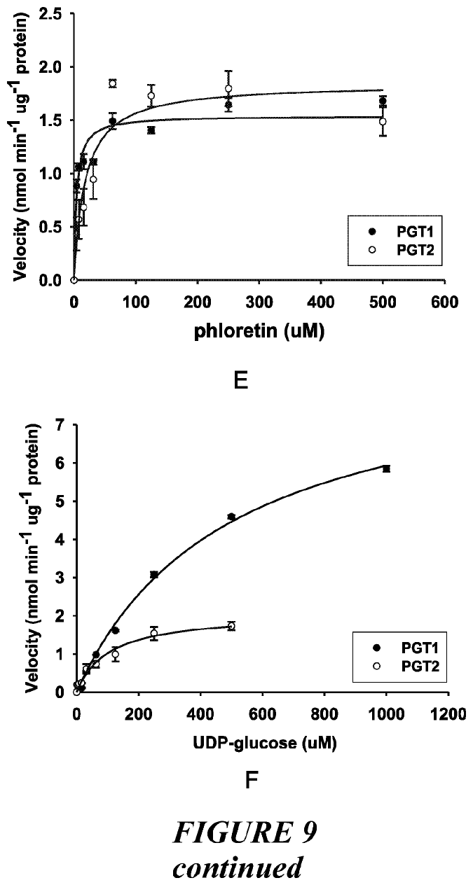

Activity of PGT2 (A) and PGT1 (B) were tested at 37° C., over the pH range 4-12, using three buffer systems: Black line=0.1 M Na-citrate buffer at pH 4.0, 5.0, 6.0; 0.1 M Tris-HCl buffer at pH 7.0, 8.0, 9.0, 10.0 and 0.1 M Na₂HPO₄-NaOH at pH 11.0, 12.0. Red line=Glycine-NaOH buffer at pH 8.6, 9.0, 10.0, 10.6. Green line=Britton-Robinson buffer at pH 6.0-11.0. The pH optima of both enzymes was between pH 8.0-9.0 (FIG. 9A, B).

The temperature-dependent activity of PGT2 and PGT1 are shown in (C) and (D) respectively. The Kₘ values of phloretin (E) were determined at concentrations from 4-500 μM at a fixed UDP-glucose concentration of 500 μM. The optimum temperature was ˜40° C. (FIG. 9C, D).

The Kₘ values of UDP-glucose (F) were determined at concentrations from 2-500 μM with a fixed phloretin concentration of 500 μM. The Km values of PGT2 for phloretin were 18.0+6.7 μM (Vmax=1.85+0.17 nmol·min−1) and for UDP-glucose 103.6+23.0 μM: (Vmax=2.07+0.17 nmol·min−1) (FIG. 9E, F). These Km values are comparable to those obtained for PGT1 for phloretin of 4.1+1.2 μM (Vmax=1.54+0.08 nmol·min−1) and for UDP-glucose of 491+41 μM (Vmax=8.84+0.35 nmol·min−1) under the same purification conditions (FIG. 9E, F).

2.3 Discussion

In this Example the inventors further show that PGT2 is responsible for trilobatin biosynthesis. They also show that PGT2 can be expressed in E. coli and produce an enzyme with 4-O-glycosyltransferase activity, and that this enzyme can produce trilobatin when contacted with phloretin and UDP-glucose.

### 3. Example 3—Structural Analysis

3.1 Materials and Methods

The sequences for PGT1-3 were independently submitted to the iTASSER server (Yang and Zhang, 2015). C-scores of the best models used for structural analysis were −0.38, 0.94 and 1.52 for PGT1, 2 and 3, respectively. Superimposition, structural analysis and figures were performed using the PyMOL Molecular Graphics System, Version 2.0 (Schrödinger, LLC, 2015).

3.2 Results

To investigate the structural basis for the difference in positional specificity for UDP-glucose, structural homology models were independently obtained for PGT1-3 using the iTASSER server (Yang and Zhang, 2015). The models were superimposed and compared with the crystal structure of UGT72B1 bound with UDP-glucose (donor) and 2,4,5-trichlorophenol (TCP, acceptor; Brazier-Hicks et al., 2007; PDB entry 2VCE). Overall, all structures were very similar, with RMSDs of ˜1 Å between each other. Sequence identity between PGT2/PGT1, PGT2/PGT3, and PGT1/PGT3 are 48, 86 and 47%, respectively. Around the predicted UDP binding site, however, the amino acid conservation between the three enzymes was much higher (>95% identity) (Table 8), consistent with the ability of these enzymes to bind the same donor molecule. Furthermore, the positions of the catalytic dyad residues in the models (His16/Asp118 in PGT2 and PGT3, His15/Asp118 in PGT1) were in excellent agreement with the crystal structure of UGT72B1. In contrast, the amino acid conservation between PGT2 and PGT1 was considerably lower among the 13 amino acids shaping the acceptor binding pocket (23% identity; 3 residues). Similarly, although less pronounced, the amino acid conservation between PGT2 and PGT3 around the acceptor binding pocket dropped to 69% (9 residues).

TABLE 8
Amino acids surrounding the donor and acceptor binding
sites in PGT1-PGT3, identified from the respective
3D models. Residues highlighted in bold in PGT1
and PGT3 are different when compared to PGT2.

| Binding site | PGT1 | PGT2 | PGT3 |
|---|---|---|---|
| Uridine | Trp359 | Trp349 | Trp350 |
| | Ala360 | Ala350 | Ala351 |
| | Pro361 | Pro351 | Pro352 |
| | Gly289 | Gly279 | Gly279 |
| | Phe285 | Phe275 | Phe275 |
| | Cys287 | Cys277 | Cys277 |
| | Gln362 | Gln352 | Gln353 |
| | Glu385 | Glu375 | Glu376 |
| | Val17 | Val18 | Ile18 |
| Diphosphate | Gly14 | Gly15 | Gly15 |
| | Ser382 | Ser372 | Ser373 |
| | Asn381 | Asn371 | Asn372 |
| | His377 | His367 | His368 |
| | Gly379 | Gly369 | Gly370 |
| | Ser290 | Ser280 | Ser280 |
| | Tyr399 | Tyr389 | Tyr390 |
| Glucose | His15 | His16 | His16 |
| | Asp118 | Asp118 | Asp118 |
| | Ser141 | Ser141 | Ser141 |
| | Glu401 | Glu391 | Glu392 |
| | Gln402 | Gln392 | Gln393 |
| | Thr140 | Thr140 | Thr140 |
| | Met291 | Leu281 | Leu281 |
| Acceptor | Gly12 | Leu13 | Pro13 |
| | Pro85 | Glu85 | Glu85 |
| | Leu190 | Pro186 | Pro186 |
| | Ala400 | Ala390 | Ala391 |
| | Phe120 | Phe120 | Phe120 |
| | Leu119 | Phe119 | Phe119 |
| | Phe149 | Phe149 | Phe149 |
| | Val84 | His84 | His84 |
| | Thr88 | Ile88 | Thr88 |
| | Met202 | Phe198 | Phe198 |
| | Val188 | Pro184 | Ala184 |
| | Ile145 | Asn145 | Phe145 |
| | Cys139 | Phe139 | Phe139 |

3.3 Discussion

The enzymatic conversion of the same acceptor (phloretin) into two distinct isomers (phloridzin vs trilobatin) results from the ability of the enzymes to bind the acceptor inside the active site in a specific conformation, positioning the hydroxyl group of the acceptor to be glycosylated (in the 2′ and 4′ position of phloretin, respectively) in the vicinity of the catalytic histidine and of the donor sugar group. The modelling results for PGT2 and PGT1, highlighting large variations in the amino acid composition of their respective acceptor binding pocket, are consistent with the ability of these two enzymes to have different activities and to generate different products. However, due to the inherent uncertainty of the position of the individual side chains in the 3D models, further modelling of the conformations of phloretin inside the acceptor binding pocket cannot be performed. In the case of PGT3, the 3D model analysis suggests that the low enzymatic activity may be due to one (or to a combination) of the four amino acids differing with PGT2 in the acceptor binding pocket. Among these, the substitution at position 145 of an Asn in PGT2 for a bulkier Phe in PGT3 may restrict the size of the pocket and impair the binding of the acceptor. However, crystallographic work is required to confirm this hypothesis and to further understand the 2′-oGT activity of PGT3, compared to the 4′-oGT activity of PGT2.

### 4. Example 4—Metabolic Engineering of Trilobatin Production in Tobacco

4.1 Materials and Methods

PGT2 was amplified from M. trilobata, pHEX2-MdCHS and MdDBR from ‘Royal Gala’ using the following primers:

Cloning

| | Forward primer | | Reverse primer | |
|---|---|---|---|---|
| Name | (5′→3′) | Name | (5′→3′) | Purpose |
| 28a-88A1F | TAAGAAGGAGATATACCATGG | 28a-88A1R | GTGGTGGTGGTGGTGCTC | Cloning PGT2 |
| | AGGCGACAGCTATAGTTTTA | | GAGACAGGTTTTGCCCCA | (M. toringoides-2) into |
| | | | GAATTCA | pET28a(+) for expression |
| | | | | in E. coli |
| 28a-88F1F | TAAGAAGGAGATATACCATGG | 28a-88F1R | GTGGTGGTGGTGGTGCTC | Cloning PGT1 |
| | GAGACGTCATTGTACTGTA | | GAGTGTTATGCTATTAAC | (M. x domestica ‘Fuji’) |
| | | | AAAGTTGACCAA | into pET28a(+) |
| 28a-304F | TAAGAAGGAGATATACCATGG | 28a-304R | GTGGTGGTGGTGGTGCTC | Cloning PGT3 |
| | ATGGAGGCGGCAGCTATAGTT | | GAGCTAACCAGTTTTGCC | (M. sieboldii-2) |
| | TTATA | | CACAATTGAA | into pET28a(+) |

Genes were cloned into pHEX2 to generate the binary vectors pHEX2-PGT2, pHEX2-CHS and pHEX2-DBR respectively. Construction of pHEX2-Myb10, pBIN61-p19 (containing the suppressor of gene silencing p19) and the control construct pHEX2-GUS have been reported previously (Voinnet et al., 2003; Espley et al., 2007; Nieuwenhuizen et al., 2013). All constructs were electroporated in Agrobacterium tumefaciens strain GV3101. Freshly grown cultures were mixed in equal ratio and infiltrated into N. benthamiana leaves as described in Hellens et al., (2005). After 7 d, leaves were harvested and phenolic compounds extracted for Dionex-HPLC analysis.

4.2 Results

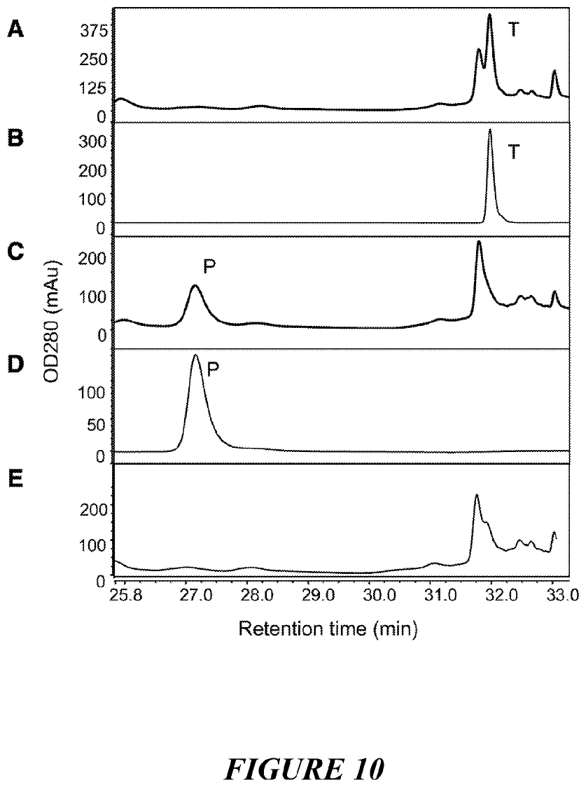

To reconstitute the full apple pathway for trilobatin and phloridzin production in Nicotiana benthamiana, MdMyb10 and two biosynthetic genes MdDBR and MdCHS were transiently expressed together to catalyze the synthesis of phloretin substrate for glycosylation. The MdMyb10 transcription factor was required to increase substrate flux through the phenylpropanoid pathway. Leaves infiltrated with MdMyb10, MdDBR, MdCHS and PGT2 were analyzed by Dionex-HPLC and exhibited a peak at 32 min that corresponded to the trilobatin standard (FIG. 10A, B), whilst those infiltrated with PGT1 exhibited a peak at 27.2 min corresponding to phloridzin (FIG. 10C, D). Neither phloridzin nor trilobatin were detected in leaves inoculated with a GUS control vector (FIG. 10E). These results indicate that three biosynthetic genes and a transcription factor are sufficient to reconstitute the pathway to trilobatin and phloridzin production in tobacco (and likely any other plant) for biotechnological applications.

4.3 Discussion

In this example the inventors show that the 4′-oGT activity and trilobatin content of plants can be increased by expression of PGT2.

Identification of the 4′-oGT for trilobatin production and reconstitution of the apple pathway to trilobatin and phloridzin production in tobacco can allow high levels of trilobatin to be produced via biotechnological means such by biopharming and metabolic engineering in yeast. The utility of this approach has already been demonstrated for PGT1 in yeast (Eichenberger et al., 2017), but not in planta. The ability to produce large quantities of trilobatin would allow it to be tested as a natural sweetener in the food and beverage industry but also for its potential health benefits (Fan et al., 2015; Xiao et al., 2017).

### 5. Example 5—Over-Expression of PGT2 in M. x Domestica and Sensory Evaluation

5.1 Materials and Methods

5.1.1 Generation of transgenic apple plants The coding region of PGT2 was amplified from M. toringoides using the primers below and cloned into pCAMBIA2300 using the One Step Cloning Kit (www.vazyme.com):

Cloning

| | Forward primer | | Reverse primer | |
|---|---|---|---|---|
| Name | (5′→3′) | Name | (5′→3′) | Purpose |
| 2300- | GAGAACACGGGGGACTCTAGA | 2300- | GTGGTCCTTGTAATCGGTACC | Cloning PGT2 |
| 88A1F | ATGGAGGCGACAGCTATAGT | 88A1R | CTAACAGGTTTTGCCCCAGAA | (M. toringoides-2) into |
| | | | T | pCambia2300 for |
| | | | | transformation of ‘GL3’ |
| MT2GTF | [symbol] CATGGA | MT2GTR | [symbol] CTAACCAG | Gateway cloning PGT2 |
| | GGCGGCAGCTATAGT | | TTTTGCCCCACA | (M. trilobata-2) into pHEX2 for |
| | | | | transient expression and |
| | | | | transformation of |
| | | | | M. x domestica ‘Royal Gala’ |
| CHS1F | [symbol] CATGGT | CHS1R | [symbol] TCAAGCAC | Cloning MdCHS |
| | GACCGTCGAAGAAGT | | CCACACTGTGAA | (AAY45748) into pHEX2 for |
| | | | | transient expression |

| | |
|---|---|
| MdDBR | MdDBR was cloned directly from EST EB156073 by digestion with SpeI + XhoI into |
| | the corresponding sites of pSAK778 for transient expression |

The PGT2:pCAMBIA plasmid was then transformed to Agrobacterium tumefaciens (strain GV3101) cells. Transgenic ‘GL3’ apple plants were generated by Agrobacterium-mediated transformation according to Dai et al. (2013) and Sun et al. (2018). Transgenic ‘Royal Gala’ plants were transformed with pHEX2-PGT2 and plants regenerated as described by Yao et al. (1995, 2013).

PGT2 expression levels and dihydrochalcone content in transgenic ‘GL3’ apple lines were determined using qRT-PCR and HPLC. The relative expression of PGT2 in fourteen transgenic ‘GL3’ lines (#) was determined by qRT-PCR using RNA extracted from young leaves. Expression was corrected against Mdactin and is given relative to the wildtype (WT) ‘GL3’ control (value set at 1). Primers and product sizes are given in Table 2. Phenolic compounds were extracted from young leaves into a solution containing 50% methanol and 2% formic acid and individual DHC content determined by HPLC.

5.1.2 Sensory Panel Analysis

Apple leaves from wildtype and two PGT2 transgenic ‘GL3’ lines were washed with water and dried at room temperature. Leaves were held at 200° C. for 1 min to inactivate enzymes, then dried at 80° C. in an oven for 60 min. Apple leaf tea was made using 5 g of dried leaves with the ratio of leaves:water being 1:100 (g:ml). Water at ˜80° C. was added to the leaves for 15 min, then all leaves were removed to stop further extraction. The tea was then kept at 50° C. in water bath for sensory analysis. The sensory panel consisted of 23 individuals and included 14 females and 9 males (all 20-30 years of age). Participation was voluntary and all participants gave their written consent prior to participation in the study. For the triangle tests, participants were given three trays, each tray had three cups (2 ml tea in each cup) where transgenic and wildtype leaf tea were in a random design, either two transgenic and one wildtype or two wildtype and one transgenic. Participants were asked to sequentially taste the three samples on each tray and select which sample was different. To assess the relative sweetness of wildtype vs transgenic apple leaf teas, two samples (one transgenic and one wildtype) were presented and the 23 panelists were asked to score the two samples on an unanchored scale sweetness scale from 1-10. For all the tasting tests, participants kept the samples in their mouths for 1-2 seconds, then spat them out into a waste container. Participants rinsed their mouths between samples with water and a dry biscuit was provided between each sample set.

Five participants with high acuity for trilobatin in the triangle test were selected to perform the isosweetness comparison test between trilobatin and sucrose. Each participant was given one trilobatin solution and eight sucrose solutions at different concentrations to taste. Solutions were prepared as described above for the apple leaf teas. The trilobatin solutions were presented at 12.3, 18.5, 27.8 and 41.7 mg per 100 ml, while the sucrose solutions were presented at 296.3, 444.4, 592.6, 666.7, 888.9, 1000, 1333.3 and 2000 mg per 100 ml.

5.2 Results

5.2.1 Over-expression in M. x domestica

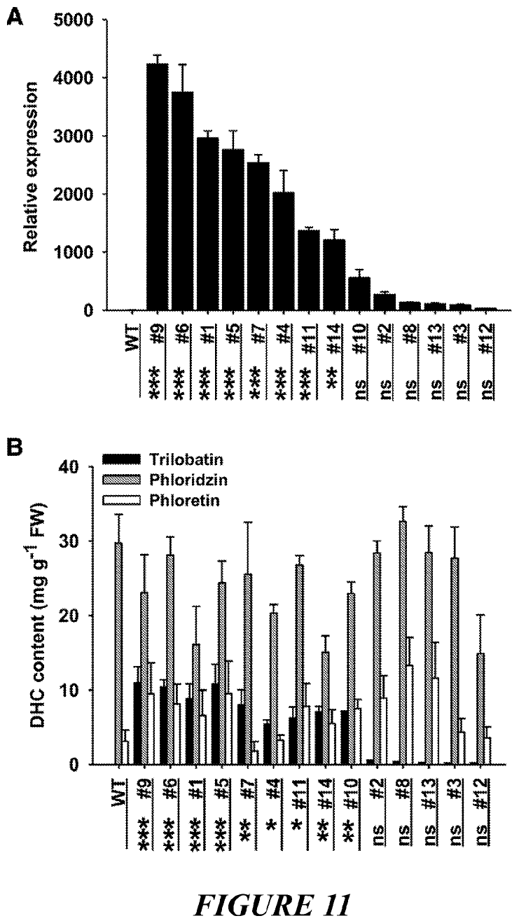

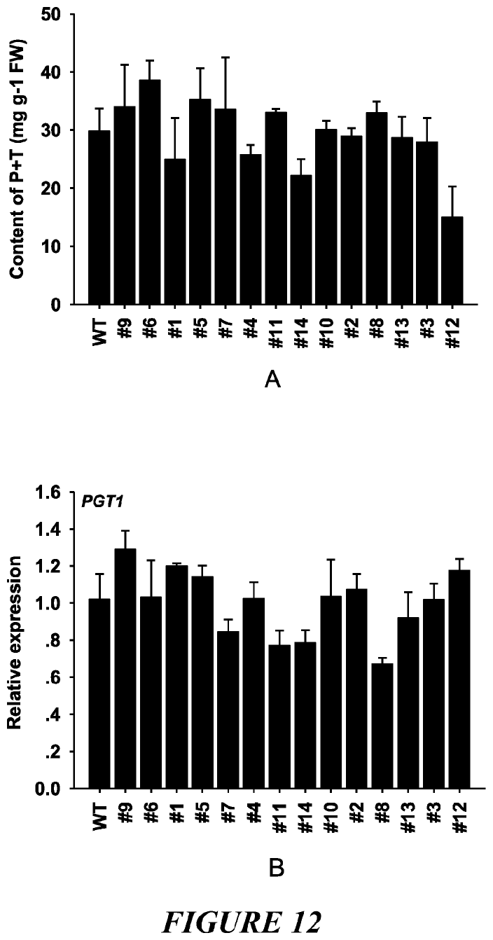

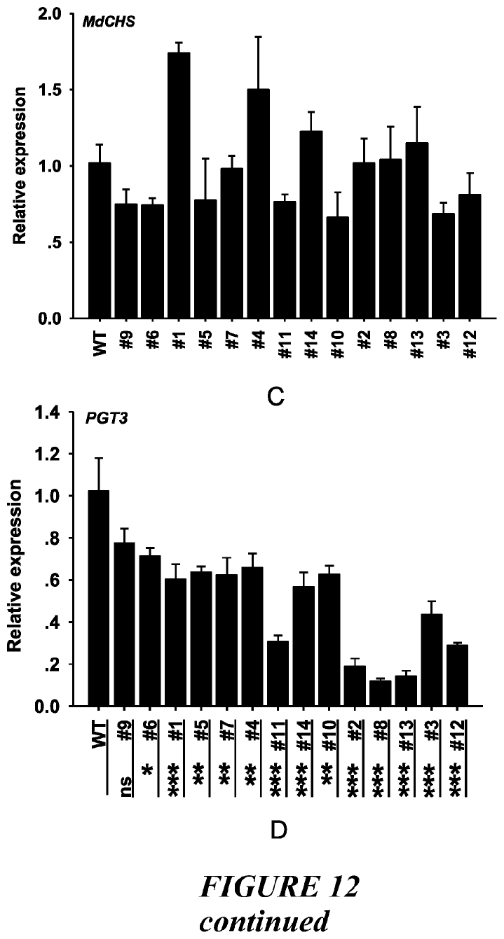

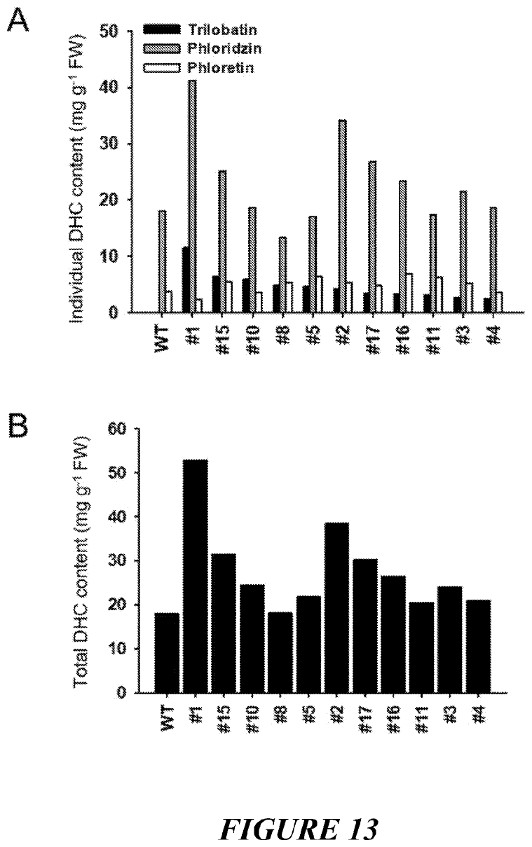

PGT2 was over-expressed in two M. x domestica backgrounds ‘GL3’ and ‘Royal Gala’. Fourteen transgenic ‘GL3’ lines were obtained and PGT2 expression was significantly increased in the leaves of 4 week old plants from eight lines (#'s 1, 4, 5, 6, 7, 9, 11, 14) compared to wildtype (FIG. 11A). Levels of trilobatin were significantly increased in the same eight lines+line #10 compared to wildtype, with levels ranging from 5.4-11.0 mg·g⁻¹ FW (FIG. 11B). No significant differences were observed in phloridzin, phloretin (FIG. 11B) or total content of trilobatin and phloridzin (FIG. 12A) among the ‘GL3’ transgenic lines. Eleven transgenic ‘Royal Gala’ lines over-expressing PGT2 were also regenerated and shown to contain increased levels of trilobatin compared to wildtype, with levels ranging from 3-11 mg·g⁻¹ FW (FIG. 13A) and with similar total content of trilobatin and phloridzin (FIG. 13B).

The relative expression of PGT1, MdCHS and PGT3 were also analyzed by qRT-PCR in the ‘GL3’ transgenic PGT2 over-expression lines. The expression levels of PGT1 (FIG. 12B) and MdCHS (FIG. 12C) were not significantly altered in the 14 transgenic apple lines. Interestingly, the relative expression of PGT3 in all 13/14 transgenic ‘GL3’ lines decreased significantly (FIG. 12D). Strongest suppression was observed in lines expressing PGT2 and trilobatin at the lowest levels suggesting co-suppression of the endogenous PGT3 gene by the introduced PGT2 transgene.

5.2.2 Sensory Evaluation of Apple Leaf Teas from PGT2 Transgenic Plants

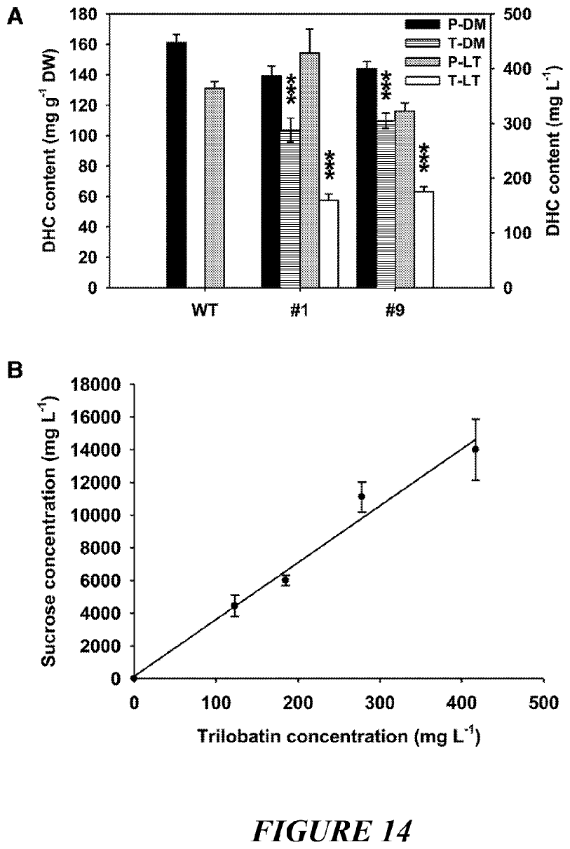

Sensory analysis was used to investigate the impact of PGT2 over-expression on the taste of apple leaf tea. Leaves were harvested from 4 month old wildtype ‘GL3’ plants and two transgenic lines (#'s 1, 9). After drying, the phloridzin content in the wildtype and transgenic leaves were similar (˜150 mg·g⁻¹ DW). The transgenic lines also contained trilobatin (˜100 mg·g⁻¹ DW), whilst the wildtype contained none (FIG. 14A). After steeping, ˜27% of the phloridzin and 16% of the trilobatin was extracted into the tea (FIG. 14A).

In triangle tests, panelists were clearly able to distinguish the flavor of tea produced from the transgenic PGT2 leaves compared to tea produced from wildtype (p<0.01, n=70 observations). To determine the basis for this discrimination, panelists were then asked to rate the sweetness of each sample on an unanchored sweetness scale (1-10). The average sweetness of the two transgenic lines was rated significantly (p<0.05, n=23) higher at 4.8 and 4.6 respectively, compared to that of wildtype rated at 3.2.

The isosweetness comparison test between trilobatin purified from leaves of the crabapple hybrid ‘Adams’ and sucrose indicated that trilobatin was ˜35-fold sweeter than sucrose (FIG. 14B). This number is slightly lower than figures reported previously (Jia et al., 2008), which may relate to purity of the trilobatin tested, the delivery system, or variation in panelist sensitivity to sucrose or trilobatin.

5.3 Discussion

Sensory analysis of apple leaf teas made from transgenic plants over-expressing PGT2 demonstrated that they could be clearly distinguished from teas made from wildtype apple leaves. The levels of trilobatin extracted into tea (FIG. 14A, ˜150 mg·L⁻¹) are above the sweetness detection threshold reported for trilobatin (3-200 mg·L⁻¹; Jia et al., 2008). The perception of increased sweetness in the transgenic leaf teas is consistent with increased production of trilobatin and not a decrease in levels of bitter tasting phloridzin.

The production of both trilobatin and phloridzin in the leaves of the transgenic plants indicates that PGT2 is reasonably competitive with PGT1 for the pool of phloretin substrate available in leaves, and that PGT2 should also be competitive with PGT1 for the smaller pool of phloretin produced in fruit. It is expected that when the transgenic ‘Royal Gala’ plants reach maturity, a sensory analysis of apple fruit from the transgenic plants with PGT2 over-expression in the fruit should also demonstrate that the fruit can be distinguished from apple fruit from wildtype plants.

Preferred embodiments of the invention have been described by way of example only and modifications may be made thereto without departing from the scope of the invention.

### 6. Example 6—Production of Trilobatin in Escherichia coli

6.1 Materials and Methods

6.1.1 Cloning

The coding sequence of PGT2-2 from Malus toringoides (SEQ ID NO. 11; NCBI accession number MN381000; Wang et al., 2020) was codon optimised for E. coli in GeneArt and synthesised by TWIST Bioscience USA (https://www.twistbioscience.com/). The PGT2-2 coding sequence was cloned into pCDFDuet™−1 (Novagen, USA) by restriction/ligation cloning using the EcoRV and KpnI restriction sites.

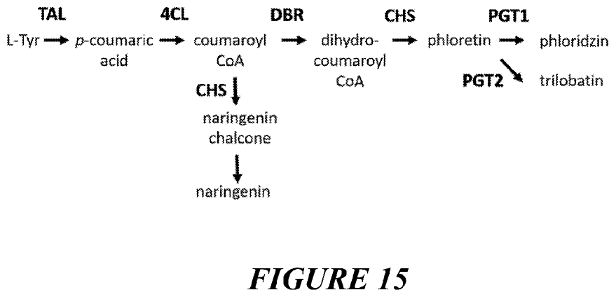

The other components of the trilobatin production pathway (FIG. 15) were cloned into co-expression plasmids using the same method, as shown in Table 9.

TABLE 9
Plasmid constructs for expression in E. coli.

| Plasmid | | | |
|---|---|---|---|
| no. | Plasmid | Gene 1 | Gene 2 |
| 1 | pRSFDuet ™-1 | TAL (tyrosine ammonia lyase) | 4CL (4-coumarate-CoA ligase) |
| | | from Rhodotorula glutinis | from Solarium lycopersicum |
| | | | (GenBank: AK328438) |
| 2 | pCDFDuet ™-1 | CHS2 (chalcone synthase 2) | PGT2 (phloretin 4′-O- |
| | | from Hordeum vulgare | glycosyltransferase) from |
| | | (GenBank: Y09233) | Malus toringoides |
| | | | (GenBank: MN381000) |
| 3 | pETDuet ™-1 | ErED (enoate reductase) | |
| | | from Eubacterium ramulus | |
| | | (GenBank: AGS82961) | |
| 4 | pETDuet ™-1 | TSC13 (very-long-chain | |
| | | enoyl-CoA reductase) from | |
| | | Saccharomyces cerevisiae | |
| | | (GenBank: NM_001180074.1) | |

Plasmid DNA was extracted by NucleoSpin ® Plasmid kit (Macherey-Nagel, Germany) and sequenced by Sanger sequencing.

6.1.2 Expression

BL21(DE3) electrocompetent E. coli cells were co-transformed with plasmids 1 and 2, providing all of the trilobatin metabolic pathway except for the double bond reductase, and either plasmid 3 or 4 to provide the double bond reductase (Table 9 and FIG. 15). Control strains were also obtained—C-1′ lacking a double bond reductase; C-2 lacking a double bond reductase and PGT2; and C-3 lacking a double bond reductase, PGT2, and CHS2.

Bacteria strains were grown in LB (1% w/v tryptone, 0.5% w/v yeast extract, 1% w/v NaCl) at 370C, 180 rpm until OD₆₀₀=0.7. Then, 1 mM isopropyl-D-thiogalactopyranoside (IPTG) was added to induce gene expression and protein accumulation, and cultures were grown at 280C, 100 rpm for 16 h. Cells were harvested by centrifugation and resuspended in the same volume (20 mL) of terrific broth (1.2% w/v tryptone, 2.4% w/v yeast extract, 0.4% v/v glycerol, 0.17 M KH₂PO₄, 0.72 M K₂HPO₄) supplemented with 1 mM IPTG and 250 μM L-tyrosine. Feeding with L-tyrosine was conducted at 28° C., 100 rpm for 4 h until metabolite extraction.

6.1.3 Metabolite Extraction and Analysis

E. coli BL21(DE3) cultures (1 mL) were extracted with an equal volume of ethyl acetate (EtOAc) by mixing for 1 min, followed by centrifugation at 16,000 g for 2 min. The EtOAc phase was removed and the remaining lower aqueous phase was re-extracted as before. Supernatants were collected and the ethyl acetate was evaporated by incubation for 1 h 30 min at 30° C. under negative pressure in an Eppendorf Concentrator Plus™. Pellets were resuspended in 200 μL 80% v/v methanol and stored at 4° C.

Metabolite analysis was conducted on an UHPLC/QqQ-MS/MS system, as previously reported (Vrhovsek et al., 2012). 2 μL were injected and concentrations were calculated by calibration curves with authentic standards. Samples were analysed in triplicate.

6.2 Results

Two different double bond reductases, ERED and TSC13, were tested for their ability to function as part of a trilobatin production pathway in E. coli.

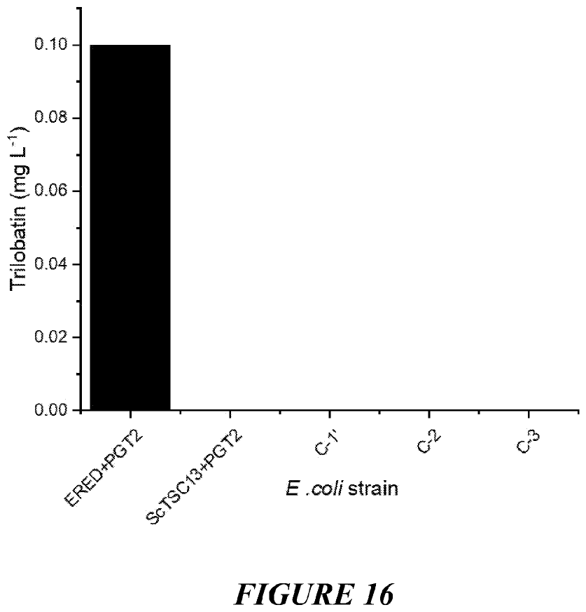

Co-expression in E. coli of TAL, 4CL, CHS2, ERED, and PGT2 resulted in the production of trilobatin (Table 10; FIG. 16). The use of S. cerevisiae TSC13 as the double-bond reductase instead of ERED did not result in any detectable trilobatin production.

Control experiments that lacked a double-bond reductase did not produce any detectable trilobatin (C-1′ in Table 10 and FIG. 16). Neither did controls lacking a double bond reductase and PGT2, or controls lacking a double bond reductase, PGT2, and CHS2 (C-2 and C-3 respectively in Table 10 and FIG. 16).

TABLE 10
Metabolite production in E. coli.

| | p-coumaric | | | | |
|---|---|---|---|---|---|
| Expression | acid | Phloretin | Phloridzin | Trilobatin | Naringenin |
| construct | (mg/L) | (mg/L) | (mg/L) | (mg/L) | (mg/L) |
| ERED + PGT2* | 3.46 ± 0.11 | 0.5 ± 0 | 0.53 ± 0.05 | 0.1 ± 0 | 0.3 ± 0 |
| TSC13 + PGT2* | 1.1 ± 0.1 | 0 ± 0 | 0 ± 0 | 0 ± 0 | 0 ± 0 |
| TSC13 + PGT1* | 0.8 ± 0 | 0 ± 0 | 0.06 ± 0.11 | 0 ± 0 | 0 ± 0 |
| C-1′ | 3.06 ± 0.11 | 0 ± 0 | 0 ± 0 | 0 ± 0 | 0.96 ± 0.05 |
| C-2 | 3.53 ± 0.05 | 0 ± 0 | 0 ± 0 | 0 ± 0 | 2.03 ± 0.05 |
| C-3 | 6.13 ± 0.05 | 0 ± 0 | 0 ± 0 | 0 ± 0 | 0 ± 0 |

*Also expressed TAL, 4CL, and CHS2.

6.3 Discussion

In this Example, the inventors show that genes involved in the trilobatin production pathway can be expressed in E. coli, and that trilobatin can be produced by E. coli grown in culture.

### 7. Example 7—Production of Trilobatin in Saccharomyces cerevisiae

7.1 Materials and Methods

7.1.1 Cloning

The coding sequence of PGT2-2 from Malus toringoides (SEQ ID NO. 11; NCBI accession number MN381000; Wang et al., 2020) was codon optimised for S. cerevisiae in GeneArt and synthesised by TWIST Bioscience USA (https://www.twistbioscience.com/). The PGT2-2 coding sequence was cloned into pAT425 (Ishii et al., 2014) by restriction/ligation cloning using the Sa/I and NotI restriction sites.

Ligated plasmids were transformed into E. coli TOP10 cells and sequenced as described in Example 6 section 6.1.1.

7.1.2 Expression

A uracil auxotrophic strain of Saccharomyces cerevisiae producing phloretin, harbouring HaCHS (Hypericum androsaemum; UniProt: Q9FUB7.1), ScTSC13 (Saccharomyces cerevisiae; GenBank: NM_001180074.1), At4CL2 (Arabidopsis thaliana; GenBank: NP_188761.1), AtPAL2 (Arabidopsis thaliana; GenBank: NP_190894), AmC4H (Ammi majus; GenBank: AA062904.1) and ScCPR1 (Saccharomyces cerevisiae; GenBank: NP_011908), was transformed with the PGT2-2-containing vector or the empty vector according to Gietz and Schiestl (2007), plated in synthetic drop-out (SD) media (Sigma-Aldrich, Germany) without uracil and leucine (SD-U-L) and incubated at 30° C. for 3 days. Then, a single transformant colony was grown overnight in 5 mL SD-U-L at 300C, 200 rpm and used to inoculate 50 mL SD-U-L. Yeast cultures were grown at 300C, 200 rpm for 48 and 72 h.

7.1.3 Metabolite Analysis

Metabolite analysis was performed as described in Example 6, section 6.1.3.

7.2 Results

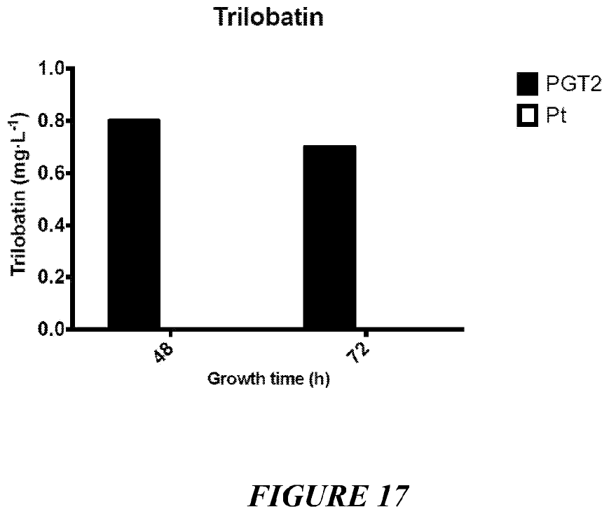

The production of trilobatin by S. cerevisiae was determined at 48 and 72 hours. Trilobatin production was detectable at both time-points for the PGT2-2 expression strain, and no production was detected for the phloretin strain control (Table 11 and FIG. 17).

TABLE 11
Metabolite production in S. cerevisiae.

| | | p-coumaric | | | |
|---|---|---|---|---|---|
| | | acid | Phloretin | Phloridzin | Trilobatin |
| Strain | OD₆₀₀ | (mg/L) | (mg/L) | (mg/L) | (mg/L) |
| PGT2-2 48 h | 0.406 | 56.33 ± 0.64 | 1.73 ± 0.12 | 4.40 ± 0.10 | 0.80 ± 0.00 |
| PGT2-2 72 h | 0.371 | 60.60 ± 1.25 | 0.90 ± 0.10 | 3.43 ± 0.21 | 0.70 ± 0.00 |
| Pt 48 h | 0.212 | 1.70 ± 0.20 | 0.37 ± 0.06 | nd | nd |
| Pt 72 h | 0.217 | 1.67 ± 0.15 | 0.10 ± 0.00 | nd | nd |

nd = not detected.
Pt = phloretin strain control.

7.3 Discussion

In this Example, the inventors show that the genes involved in the trilobatin production pathway can be expressed in S. cerevisiae, and that trilobatin can be produced by S. cerevisiae when grown in culture.

### SUMMARY OF SEQUENCES

TABLE 12
PGT2 sequences.

| SEQ | Molecule | | | GenBank |
|---|---|---|---|---|
| ID NO. | type | Species | Classification | Accession No. |
| 1 | polypeptide | Malus | phloretin 4′-O- | MN380999 |
| | | toringoides | glycosyltransferase | |
| | | | (MtgPGT2-1) | |
| 2 | polypeptide | Malus | phloretin 4′-O- | MN381000 |
| | | toringoides | glycosyltransferase | |
| | | | (MtgPGT2-2) | |
| 3 | polypeptide | Malus sieboldii | phloretin 4′-O- | MN381001 |
| | | | glycosyltransferase | |
| | | | (MsPGT2-1) | |
| 4 | polypeptide | Malus ‘Adams’ | phloretin 4′-O- | MN381002 |
| | | | glycosyltransferase | |
| | | | (AdamsPGT2-1) | |
| 5 | polypeptide | Malus | phloretin 4′-O- | MN381003 |
| | | domestica | glycosyltransferase | |
| | | | (MdPGT2-1) | |
| 6 | polypeptide | Malus trilobata | phloretin 4′-O- | MN381004 |
| | | | glycosyltransferase | |
| | | | (MtbPGT2-1) | |
| 7 | polypeptide | Malus trilobata | phloretin 4′-O- | MN381005 |
| | | | glycosyltransferase | |
| | | | (MtbPGT2-2) | |
| 8 | polypeptide | Malus ‘Aotea’ | phloretin 4′-O- | MN381006 |
| | | | glycosyltransferase | |
| | | | (AoteaPGT2-1) | |
| 9 | polypeptide | Malus | Predicted UDP- | Cazyme ID |
| | | domestica | glucosyl transferase | MDP0000836043 |
| | | | 88A1 | |
| 10 | polynucleotide | Malus | phloretin 4′-O- | MN380999 |
| | | toringoides | glycosyltransferase | |
| | | | (MtgPGT2-1) | |
| 11 | polynucleotide | Malus | phloretin 4′-O- | MN381000 |
| | | toringoides | glycosyltransferase | |
| | | | (MtgPGT2-2) | |
| 12 | polynucleotide | Malus sieboldii | phloretin 4′-O- | MN381001 |
| | | | glycosyltransferase | |
| | | | (MsPGT2-1) | |
| 13 | polynucleotide | Malus | phloretin 4′-O- | MN381002 |
| | | | glycosyltransferase | |
| | | | (AdamsPGT2-1) | |
| 14 | polynucleotide | Malus | phloretin 4′-O- | MN381003 |
| | | domestica | glycosyltransferase | |
| | | | (MdPGT2-1) | |
| 15 | polynucleotide | Malus trilobata | phloretin 4′-O- | MN381004 |
| | | | glycosyltransferase | |
| | | | (MtbPGT2-1) | |
| 16 | polynucleotide | Malus trilobata | phloretin 4′-O- | MN381005 |
| | | | glycosyltransferase | |
| | | | (MtbPGT2-2) | |
| 17 | polynucleotide | Malus | phloretin 4′-O- | MN381006 |
| | | | glycosyltransferase | |
| | | | (AoteaPGT2-1) | |
| 18 | polynucleotide | Malus | Predicted UDP- | Cazyme ID |
| | | domestica | glucosyl transferase | MDP0000836043 |
| | | | 88A1 | |

SEQUENCE LISTING:

>SEQ1 [organism = Malus toringoides] phloretin 4′-O-glycosyltransferase (MtgPGT2-1),
polypeptide
MEATAIVLYPSPLIGHLVSMVELGKLILTRHPSLCIHILITTPPYRANDTDSYITSVSAANPSLIFHHLPTISLPPS
LASSRNHEILAFELAPLYNPNVHQALVSISHNFSIKAFVMDFFCYVGLPVATELNIPSYFFFTSSANTLASSLYLPT
LHNIIDKSLKDLNILLNIPGVPPMPSSDMPQPTLDRNQKVYEHVQGSSKQFPKSAGIIVNTFESLEPRALRAIWDGL
CLPENVPTPPVYPIGPLIVSHGGGGRGAECLKWLDSQPSGSVVFLCFGSLGLFSKEQLKEIAIGLENSGHRFLWVVR
NPPAQNQIGLDIKESDPELKSLLPDGFLDRTKDRGLVVKSWAPQAAVLNHNSVGGFVSHCGWNSVLESVCAGVPIVA
WPLYAEQRFNRVVLVKEIKIAMPMNESEDGFVRAAEVEKRITELMDSEEGASIRKRTKDLQNNAHAALGETGSSGVA
LTKLLELWGKTC*
>SEQ2 [organism = Malus toringoides] phloretin 4′-O-glycosyltransferase (MtgPGT2-2),
polypeptide
MEATAIVLYPSPLIGHLVSMVELGKLILTRHPSLCIHILITTPPYRANDTDSYITSVSAANPSLIFHHLPTISLPPS
LASSRNHEILAFELAPLYNPNVHQALVSISHNFSIKAFVMDFFCYVGLPVATELNIPSYFFFTSSANTLASSLYLPT
LHNIIDKSLKDLNILLNIPGVPPMPSSDMPQPTLDRNQKVYEHVQGSSKQFPKSAGIIVNTFESLEPRALRAIWDGL
CLPENVPTPPVYPIGPLIVSHGGGGRGAECLKWLDSQPSGSVVFLCFGSLGLFSKEQLKEIAIGLENSGHRFLWVVR
NPPAQNQIGLDIKESDPELKSLLPDGFLDRTKDRGLVVKSWAPQAAVLNHNSVGGFVSHCGWNSVLESVCAGVPIVA
WPLYAEQRFNRVVLVKEIKIAMPMNESEDGFVRAAEVEKRITELMDSEEGASIRKRTKDLQNNAHAALGETGSSGVA
LTKLLEFWGKTC*
>SEQ3 [organism = Malus sieboldii] phloretin 4′-O-glycosyltransferase (MsPGT2-1),
polypeptide
MEATAIVLYPSPLIGHLVSMVELGKLILTRHPSLCIHILITTPPYRANDTDSYITSVSAANPSLIFHHLPTISLPPS
LASSRNHEILAFELAPLYNPNVHQALVSISHNFSIKAFVMDFFCYVGLPVATELNIPSYFFFTSSANTLASSLYLPT
LHNIIDKSLKDLNILLNIPGVPPMPSSDMPQPTLDRNQKVYEHVQGSSKQFPKSAGIIVNTFESLEPRALRAIWDGL
CLPENVPTPPVYPIGPLIVSHGGGGRGAECLKWLDSQPSGSVVFLCFGSLGLFSKEQLKEIAIGLENSGHRFLWVVR
NPPAQNQIGLDIKESDPELKSLLPDGFLDRTKDRGLVVKSWAPQAAVLNHNSVGGFVSHCGWNSVLESVCAGVPIVA
WPLYAEQRFNRVVLVKEIKIAMPMNESEDGFVRAAEVEKRITELMDSEEGASIRKRTKDLQNNAHAALGETGSSGVA
LTKLLEFWGKTC*
>SEQ4 [organism = Malus] phloretin 4′-O-glycosyltransferase (AdamsPGT2-1), polypeptide
MEATAIVLYPSPLIGHLVSMVELGKLILTRHPSLCIHILITTPPYRANDTDSYITSVSAANPSLIFHHLPTISLPPS
LASSRNHEILAFELAPLYNPNVHQALVSISHNFSIKAFVMDFFCYVGLPVATELNIPSYFFFTSSANTLASSLYLPT
LHNIIDKSLKDLNILLNIPGVPPMPSSDMPQPTLDRNQKVYEHVQGSSKQFPKSAGIIVNTFESLEPRALRAIWDGL
CLPENVPTPPVYPIGPLIVSHGGGGRGAECLKWLDSQPSGSVVFLCFGSLGLFSKEQLKEIAIGLENSGHRFLWVVR
NPPAQNQIGLDIKESDPELKSLLPDGFLDRTKDRGLVVKSWAPQAAVLNHNSVGGFVSHCGWNSVLESVCAGVPIVA
WPLYAEQRFNRVVLVKEIKIAMPMNESEDGFVRAAEVEKRITELMDSEEGASIRKRTKDLQNNAHAALGETGSSGVA
LTKLLEFWGKTC*
>SEQ5 [organism = Malus domestica] phloretin 4′-O-glycosyltransferase (MdPGT2-1),
polypeptide
MEATAIVLYPSPLIGHLVSMVELGKLILTRHPSLCIHILITTPPYRANDTDSYITSVSAANPSLIFHHLPTISLPPS
LASSRNHEILAFELAPLYNPNVHQALVSISHNFSIKAFVMDFFCYVGLPVATELNIPSYFFFTSSANTLASSLYLPT
LHNIIDKSLKDLNILLNIPGVPPMPSSDMPQPTLDRNQKVYEHVQGSSKQFPKSAGIIVNTFESLEPRALRAIWDGL
CLPENVPTPPVYPIGPLIVSHGGGGRGAECLKWLDSQPSGSVVFLCFGSLGLFSKEQLKEIAIGLENSGHRFLWVVR
NPPAQNQIGLDIKESDPELKSLLPDGFLDRTKDRGLVVKSWAPQAAVLNHNSVGGFVSHCGWNSVLESVCAGVPIVA
WPLYAEQRFNRVVLVKEIKIAMPMNESEDGFVRAAEVEKRITELMDSEEGASIRKRTKDLQNNAHAALGETGSSGVA
LTKLLEFWGKTC*
>SEQ6 [organism = Malus trilobata] phloretin 4′-O-glycosyltransferase (MtbPGT2-1),
polypeptide
MEAAAIVLYPSPPIGHLVSMVELGKLILTRHPSLCIHILITTPPYRANDTDSYITSVSAANPSLIFHHLPTISLPPS
LASSRNHETLTFGLAPLNNPNVHQALLSISHNFSIKAFVMDFFCSVGLPIATELNIPSYFFFTSSATTLASFLYLPT
IHNITDKSLKDLNILLNIPGVPPIPSSDMPQPILERNNKVYEQCQESSKQFPKSAGIIVNTFESLEPRALRAIWDGL
CLTENVPTPPVYPIGPLIVSHGGGGRGAECLKWLDSQPSGSVVFLCFGSLGLFSKEQLKEIAIGLENSGHRFLWVVR
NPPAQNQIGLVIKESDPELKSLLPDGFLDRTKDRGLVVKSWVPQVAVLNHNSVGGFVSHCGWNSVLESVCAGVPIVS
WPLYAEQRLNRVVLVEEIKIAMPMNESEDGFVRAAEVEKRVTELMDSEEGESIRKRTKDLQNDAHAALGETGSSGVA
FTKLLELWGKTG
>SEQ7 [organism = Malus trilobata] phloretin 4′-O-glycosyltransferase (MtbPGT2-2),
polypeptide
MEAAAIVLYPSPVIGHLIAMVELGKLIITRHPSLCIHILITTPPYRANDTDSYITSVSAANPSLIFHHLPTISLPPS
LASSRNHETLTFGLAPLNNPNVHQALLSISHNFSIKAFVMDFFCSVGLPIATELNIPSYFFFTSSATTLASFLYLPT
IHNITDKSLKDLNILLNIPGVPPIPSSDMPQPILERNNKVYEQCQESSKQFPKSAGIIVNTFESLEPRALRAIWDGL
CLTENVPTPPVYPIGPLIVSHGGGGRGAECLKWLDSQPSGSVVFLCFGSLGLFSKEQLKEIAIGLENSGHRFLWVVR
NPPAQNQIGLVIKESDPELKSLLPDGFLDRTKDRGLVVKSWVPQVAVLNHNSVGGFVSHCGWNSVLESVCAGVPIVS
WPLYAEQRLNRVVLVEEIKIAMPMNESEDGFVRAAEVEKRVTELMDSEEGESIRKRTKDLQNDAHAALGETGSSGVA
FTKLLELWGKTG
>SEQ8 [organism = Malus] phloretin 4′-O-glycosyltransferase (AoteaPGT2-1), polypeptide
MEAAAIVLYPSPLIGHLVSMVELGKLILTRHPSLCIHILITTPPYRANDTDSYITSVSAANPSLIFHHLPTISLPPS
LASSRNHEILAFELAPLYNPNVHQALVSISHNFSIKAFVMDFFCYVGLPVATELNIPSYFFFTSSANTLASSLYLPT
LHNIIDKSLKDLNILLNIPGVPPMPSSDMPQPTLDRNQKVYEHVQGSSKQFPKSAGIIVNTFESLEPRALRAIWDGL
CLPENVPTPPVYPIGPLIVSHGGGGRGAECLKWLDSQPSGSVVFLCFGSLGLFSKEQLKEIAIGLENSGHRFLWVVR
NPPAQNQIGLDIKESDPELKSLLPDGFLDRTKDRGLVVKSWAPQAAVLNHNSVGGFVSHCGWNSVLESVCAGVPIVA
WPLYAEQRFNRVVLVKEIKIAMPMNESEDGFVRAAEVEKRITELMDSEEGASIRKRTKDLQNNAHAALGETGSSGVA
LTKLLELWGKTG
>SEQ9 [organism = Malus domestica] Cazyme ID: MDP0000836043. UDP-glucosyl
transferase 88A1, polypeptide
MEATAIVLYPSPLIGHLVSMVELGKLILTRHPSLCIHILITTPPYRANDTDSYITSVSAANPSLIFHHLPTISLPPS
LSPSRNHETPIFEVLLLNNPYVHQALLSISHNFSIKAFVMDFFCSVGLPIATELNIPSYFFFTSSAANLACFLYLPT
IHSITDKSLKDLNILLNIPGVQPIPSSDMPKPILERNNKVYEHFQESSKQFPKSAGIIVNTFESLEPRVLRAIWDGL
CLTENVPTPPVYPIGPLIISHGGGGRGAEYLKWLDSQPSGSVVFLCFGSLGLFSKEQLKEIAIGLENSGHRFLWVVR
NPPAQNQIGLAIKESDPELKSLLPDGFLDRTKGRGLVVKSWAPQVAVLNHNSVGGFVSHCGWNSVLESVCAGVPIVA
WPLYAEQRFNRVVLVEEIKIAMPMNESEDGFVRAAEVEKRVTELMDSEEGESIRKRTKDLQNDAHAALGETGSSRVA
FTKLLEFWGKTC
>Seq10 [organism = Malus toringoides] phloretin 4′-O-glycosyltransferase (MtgPGT2-1),
complete cds
ATGGAGGCGACAGCTATAGTTTTATATCCATCACCGCTAATTGGGCACTTAGTCTCCATGGTAGAGCTAGGCAAGCT
CATACTCACCCGCCACCCTTCTCTGTGCATCCACATCCTCATCACCACCCCGCCCTACCGCGCCAACGACACCGACT
CATACATCACCTCCGTCTCCGCTGCCAACCCTTCCCTCATCTTCCACCACCTCCCCACCATCTCCCTCCCTCCCTCC
CTCGCCTCCTCCCGCAACCACGAAATCCTAGCCTTCGAACTCGCCCCCCTCTACAACCCTAACGTCCACCAAGCCCT
CGTCTCCATCTCCCACAACTTCTCCATCAAAGCTTTTGTCATGGACTTCTTCTGCTATGTCGGGCTCCCCGTTGCCA
CCGAGCTGAACATCCCCAGCTACTTCTTCTTCACATCCAGCGCCAACACCCTCGCTTCCTCCCTCTACCTCCCCACC
CTTCACAACATCATTGACAAAAGCCTCAAAGACCTAAATATCCTTCTCAACATTCCAGGAGTCCCGCCGATGCCTTC
CTCCGATATGCCGCAACCGACTCTTGACCGAAACCAAAAAGTGTATGAACATGTCCAAGGAAGCTCAAAGCAGTTCC
CGAAATCAGCTGGGATTATCGTAAACACGTTTGAATCTCTCGAACCCAGAGCTCTCAGAGCAATATGGGACGGTCTG
TGCTTGCCCGAGAACGTTCCAACTCCACCGGTCTACCCCATCGGACCGCTGATTGTTTCCCATGGCGGTGGAGGCCG
CGGGGCCGAGTGTTTGAAATGGCTGGACTCACAGCCAAGTGGAAGCGTGGTGTTCCTCTGTTTTGGGAGCTTGGGAT
TGTTTTCAAAGGAGCAGTTGAAGGAAATTGCGATTGGGTTGGAGAATAGTGGGCACAGATTTTTGTGGGTGGTCCGT
AATCCTCCAGCCCAAAATCAAATTGGGCTGGATATTAAAGAGTCCGATCCGGAATTGAAATCTTTGCTTCCGGACGG
GTTTTTGGATCGGACTAAGGATCGGGGTCTCGTGGTCAAGTCATGGGCCCCGCAAGCGGCAGTGTTGAATCATAACT
CGGTGGGTGGGTTTGTGAGTCATTGCGGGTGGAACTCGGTGTTGGAATCGGTGTGTGCCGGTGTGCCGATTGTGGCT
TGGCCGCTCTACGCGGAGCAGAGATTCAATCGAGTGGTTTTGGTGAAGGAGATTAAGATTGCTATGCCGATGAACGA
GTCAGAAGACGGGTTTGTGAGAGCAGCGGAGGTGGAGAAGCGAATTACGGAGTTGATGGACTCGGAGGAGGGCGCGT
CGATCAGGAAGCGTACAAAGGATTTGCAAAACAATGCCCATGCAGCATTGGGTGAGACCGGGTCGTCTGGGGTTGCA
TTGACTAAACTACTTGAATTGTGGGGCAAAACCTGTTAG
>Seq11 [organism = Malus toringoides] phloretin 4′-O-glycosyltransferase (MtgPGT2-2),
complete cds
ATGGAGGCGACAGCTATAGTTTTATATCCATCACCGCTAATTGGGCACTTAGTCTCCATGGTAGAGCTAGGCAAGCT
CATACTCACCCGCCACCCTTCTCTGTGCATCCACATCCTCATCACCACCCCGCCCTACCGCGCCAACGACACCGACT
CATACATCACCTCCGTCTCCGCTGCCAACCCTTCCCTCATCTTCCACCACCTCCCCACCATCTCCCTCCCTCCCTCC
CTCGCCTCCTCCCGCAACCACGAAATCCTAGCCTTCGAACTCGCCCCCCTCTACAACCCTAACGTCCACCAAGCCCT
CGTCTCCATCTCCCACAACTTCTCCATCAAAGCTTTTGTCATGGACTTCTTCTGCTATGTCGGGCTCCCCGTTGCCA
CCGAGCTGAACATCCCCAGCTACTTCTTCTTCACATCCAGCGCCAACACCCTCGCTTCCTCCCTCTACCTCCCCACC
CTTCACAACATCATTGACAAAAGCCTCAAAGACCTAAATATCCTTCTCAACATTCCAGGAGTCCCGCCGATGCCTTC
CTCCGATATGCCGCAACCGACTCTTGACCGAAACCAAAAAGTGTATGAACATGTCCAAGGAAGCTCAAAGCAGTTCC
CTCCGATATGCCGCAACCGACTCTTGACCGAAACCAAAAAGTGTATGAACATGTCCAAGGAAGCTCAAAGCAGTTCC
TGCTTGCCCGAGAACGTTCCAACTCCACCGGTCTACCCCATCGGACCGCTGATTGTTTCCCATGGCGGTGGAGGCCG
CGGGGCCGAGTGTTTGAAATGGCTGGACTCACAGCCAAGTGGAAGCGTGGTGTTCCTCTGTTTTGGGAGCTTGGGAT
TGTTTTCAAAGGAGCAGTTGAAGGAAATTGCGATTGGGTTGGAGAATAGTGGGCACAGATTTTTGTGGGTGGTCCGT
AATCCTCCAGCCCAAAATCAAATTGGGCTGGATATTAAAGAGTCCGATCCGGAATTGAAATCTTTGCTTCCGGACGG
GTTCTTGGATCGGACTAAGGATCGGGGTCTCGTGGTCAAGTCATGGGCCCCGCAAGCGGCAGTGTTGAATCACAACT
CGGTGGGTGGGTTTGTGAGTCATTGCGGGTGGAACTCGGTGTTGGAATCGGTGTGTGCCGGTGTGCCGATTGTGGCT
TGGCCGCTCTACGCGGAGCAGAGATTCAATCGAGTGGTTTTGGTGAAGGAGATTAAGATTGCTATGCCGATGAACGA
GTCAGAAGACGGGTTTGTGAGAGCAGCGGAGGTGGAGAAGCGAATTACGGAGTTGATGGACTCGGAGGAGGGCGCGT
CGATCAGGAAGCGTACAAAGGATTTGCAAAACAATGCCCATGCAGCATTGGGTGAGACCGGGTCGTCTGGGGTTGCA
TTGACTAAACTACTTGAATTCTGGGGCAAAACCTGTTAG
>Seq12 [organism = Malus sieboldii] phloretin 4′-O-glycosyltransferase (MsPGT2-1),
complete cds
ATGGAGGCGACAGCTATAGTTTTATATCCATCACCGCTAATTGGGCACTTAGTCTCCATGGTAGAGCTAGGCAAGCT
CATACTCACCCGCCACCCTTCTCTGTGCATCCACATCCTCATCACCACCCCGCCCTACCGCGCCAACGACACCGACT
CATACATCACCTCCGTCTCCGCTGCCAACCCTTCCCTCATCTTCCACCACCTCCCCACCATCTCCCTCCCTCCCTCC
CTCGCCTCCTCCCGCAACCACGAAATCCTAGCCTTCGAACTCGCCCCCCTCTACAACCCTAACGTCCACCAAGCCCT
CGTCTCCATCTCCCACAACTTCTCCATCAAAGCTTTTGTCATGGACTTCTTCTGCTATGTCGGGCTCCCCGTTGCCA
CCGAGCTGAACATCCCCAGCTACTTCTTCTTCACATCCAGCGCCAACACCCTCGCTTCCTCCCTCTACCTCCCCACC
CTTCACAACATCATTGACAAAAGCCTCAAAGACCTAAATATCCTTCTCAACATTCCAGGAGTCCCGCCGATGCCTTC
CTCCGATATGCCGCAACCGACTCTTGACCGAAACCAAAAAGTGTATGAACATGTCCAAGGAAGCTCAAAGCAGTTCC
CGAAATCAGCTGGGATTATCGTAAACACGTTTGAATCTCTCGAACCCAGAGCTCTCAGAGCAATATGGGACGGTCTG
TGCTTGCCCGAGAACGTTCCAACTCCACCGGTCTACCCCATCGGACCGCTGATTGTTTCCCATGGCGGTGGAGGCCG
CGGGGCCGAGTGTTTGAAATGGCTGGACTCACAGCCAAGTGGAAGCGTGGTGTTCCTCTGTTTTGGGAGCTTGGGAT
TGTTTTCAAAGGAGCAGTTGAAGGAAATTGCGATTGGGTTGGAGAATAGTGGGCACAGATTTTTGTGGGTGGTCCGT
AATCCTCCAGCCCAAAATCAAATTGGGCTGGATATTAAAGAGTCCGATCCGGAATTGAAATCTTTGCTTCCGGACGG
GTTCTTGGATCGGACTAAGGATCGGGGTCTCGTGGTCAAGTCATGGGCCCCGCAAGCGGCAGTGTTGAATCACAACT
CGGTGGGTGGGTTTGTGAGTCATTGCGGGTGGAACTCGGTGTTGGAATCGGTGTGTGCCGGTGTGCCGATTGTGGCT
TGGCCGCTCTACGCGGAGCAGAGATTCAATCGAGTGGTTTTGGTGAAGGAGATTAAGATTGCTATGCCGATGAACGA
GTCAGAAGACGGGTTTGTGAGAGCAGCGGAGGTGGAGAAGCGAATTACGGAGTTGATGGACTCGGAGGAGGGCGCGT
CGATCAGGAAGCGTACAAAGGATTTGCAAAACAATGCCCATGCAGCATTGGGTGAGACCGGGTCGTCTGGGGTTGCA
TTGACTAAACTACTTGAATTCTGGGGCAAAACCTGTTAG
>Seq13 [organism = Malus] phloretin 4′-O-glycosyltransferase (AdamsPGT2-1), complete
cds
ATGGAGGCGACAGCTATAGTTTTATATCCATCACCGCTAATTGGGCACTTAGTCTCCATGGTAGAGCTAGGCAAGCT
CATACTCACCCGCCACCCTTCTCTGTGCATCCACATCCTCATCACCACCCCGCCCTACCGCGCCAACGACACCGACT
CATACATCACCTCCGTCTCCGCTGCCAACCCTTCCCTCATCTTCCACCACCTCCCCACCATCTCCCTCCCTCCCTCC
CTCGCCTCCTCCCGCAACCACGAAATCCTAGCCTTCGAACTCGCCCCCCTCTACAACCCTAACGTCCACCAAGCCCT
CGTCTCCATCTCCCACAACTTCTCCATCAAAGCTTTTGTCATGGACTTCTTCTGCTATGTCGGGCTCCCCGTTGCCA
CCGAGCTGAACATCCCCAGCTACTTCTTCTTCACATCCAGCGCCAACACCCTCGCTTCCTCCCTCTACCTCCCCACC
CTTCACAACATCATTGACAAAAGCCTCAAAGACCTAAATATCCTTCTCAACATTCCAGGAGTCCCGCCGATGCCTTC
CTCCGATATGCCGCAACCGACTCTTGACCGAAACCAAAAAGTGTATGAACATGTCCAAGGAAGCTCAAAGCAGTTCC
CGAAATCAGCTGGGATTATCGTAAACACGTTTGAATCTCTCGAACCCAGAGCTCTCAGAGCAATATGGGACGGTCTG
TGCTTGCCCGAGAACGTTCCAACTCCACCGGTCTACCCCATCGGACCGCTGATTGTTTCCCATGGCGGTGGAGGCCG
CGGGGCCGAGTGTTTGAAATGGCTGGACTCACAGCCAAGTGGAAGCGTGGTGTTCCTCTGTTTTGGGAGCTTGGGAT
TGTTTTCAAAGGAGCAGTTGAAGGAAATTGCGATTGGGTTGGAGAATAGTGGGCACAGATTTTTGTGGGTGGTCCGT
AATCCTCCAGCCCAAAATCAAATTGGGCTGGATATTAAAGAGTCCGATCCGGAATTGAAATCTTTGCTTCCGGACGG
GTTTTTGGATCGGACTAAGGATCGGGGTCTCGTGGTCAAGTCATGGGCCCCGCAAGCGGCAGTGTTGAATCATAACT
CGGTGGGTGGGTTTGTGAGTCATTGCGGGTGGAACTCGGTGTTGGAATCGGTGTGTGCCGGTGTGCCGATTGTGGCT
TGGCCGCTCTACGCGGAGCAGAGATTCAATCGAGTGGTTTTGGTGAAGGAGATTAAGATTGCTATGCCGATGAACGA
GTCAGAAGACGGGTTTGTGAGAGCAGCGGAGGTGGAGAAGCGAATTACGGAGTTGATGGACTCGGAGGAGGGCGCGT
CGATCAGGAAGCGTACAAAGGATTTGCAAAACAATGCCCATGCAGCATTGGGTGAGACCGGGTCGTCTGGGGTTGCA
TTGACTAAACTACTTGAATTCTGGGGCAAAACCTGTTAG
>Seq14 [organism = Malus domestica] phloretin 4′-O-glycosyltransferase (MdPGT2-1),
complete cds
ATGGAGGCGACAGCTATAGTTTTATATCCATCACCGCTAATTGGGCACTTAGTCTCCATGGTAGAGCTAGGCAAGCT
CATACTCACCCGCCACCCTTCTCTGTGCATCCACATCCTCATCACCACCCCGCCCTACCGCGCCAACGACACCGACT
CATACATCACCTCCGTCTCCGCTGCCAACCCTTCCCTCATCTTCCACCACCTCCCCACCATCTCCCTCCCTCCCTCC
CTCGCCTCCTCCCGCAACCACGAAATCCTAGCCTTCGAACTCGCCCCCCTCTACAACCCTAACGTCCACCAAGCCCT
CGTCTCCATCTCCCACAACTTCTCCATCAAAGCTTTTGTCATGGACTTCTTCTGCTATGTCGGGCTCCCCGTTGCCA
CCGAGCTGAACATCCCCAGCTACTTCTTCTTCACATCCAGCGCCAACACCCTCGCTTCCTCCCTCTACCTCCCCACC
CTTCACAACATCATTGACAAAAGCCTCAAAGACCTAAATATCCTTCTCAACATTCCAGGAGTCCCGCCGATGCCTTC
CTCCGATATGCCGCAACCGACTCTTGACCGAAACCAAAAAGTGTATGAACATGTCCAAGGAAGCTCAAAGCAGTTCC
CGAAATCAGCTGGGATTATCGTAAACACGTTTGAATCTCTCGAACCCAGAGCTCTCAGAGCAATATGGGACGGTCTG
TGCTTGCCCGAGAACGTTCCAACTCCACCGGTCTACCCCATCGGACCGCTGATTGTTTCCCATGGCGGTGGAGGCCG
CGGGGCCGAGTGTTTGAAATGGCTGGACTCACAGCCAAGTGGAAGCGTGGTGTTCCTCTGTTTTGGGAGCTTGGGAT
TGTTTTCAAAGGAGCAGTTGAAGGAAATTGCGATTGGGTTGGAGAATAGTGGGCACAGATTTTTGTGGGTGGTCCGT
AATCCTCCAGCCCAAAATCAAATTGGGCTGGATATTAAAGAGTCCGATCCGGAATTGAAATCTTTGCTTCCGGACGG
GTTCTTGGATCGGACTAAGGATCGGGGTCTCGTGGTCAAGTCATGGGCCCCGCAAGCGGCAGTGTTGAATCACAACT
CGGTGGGTGGGTTTGTGAGTCATTGCGGGTGGAACTCGGTGTTGGAATCGGTGTGTGCCGGTGTGCCGATTGTGGCT
TGGCCGCTCTACGCGGAGCAGAGATTCAATCGAGTGGTTTTGGTGAAGGAGATTAAGATTGCTATGCCGATGAACGA
GTCAGAAGACGGGTTTGTGAGAGCAGCGGAGGTGGAGAAGCGAATTACGGAGTTGATGGACTCGGAGGAGGGCGCGT
CGATCAGGAAGCGTACAAAGGATTTGCAAAACAATGCCCATGCAGCATTGGGTGAGACCGGGTCGTCTGGGGTTGCA
TTGACTAAACTACTTGAATTCTGGGGCAAAACCTGTTAG
>Seq15 [organism = Malus trilobata] phloretin 4′-O-glycosyltransferase (MtbPGT2-1),
complete cds
ATGGAGGCGGCAGCTATAGTTTTATATCCATCACCACCAATTGGCCACTTAGTCTCCATGGTAGAGCTAGGCAAGCT
CATACTCACCCGCCACCCTTCTCTGTGCATCCACATCCTCATCACCACCCCGCCCTACCGCGCCAACGACACCGACT
CATACATCACCTCCGTCTCCGCTGCCAACCCTTCCCTCATCTTCCACCACCTCCCCACCATCTCCCTCCCTCCCTCC
CTCGCCTCCTCCCGCAACCACGAAACCCTAACCTTCGGACTCGCCCCCCTCAACAACCCTAACGTCCACCAAGCCCT
CCTCTCCATCTCCCACAACTTCTCCATCAAAGCTTTTGTCATGGACTTCTTCTGCTCTGTCGGGCTCCCCATTGCCA
CCGAGCTGAACATCCCCAGCTACTTCTTCTTCACATCCAGCGCCACCACCCTCGCTTCCTTCCTCTACCTCCCCACC
ATTCACAACATCACTGACAAAAGCCTCAAAGACCTAAATATCCTTCTCAACATTCCAGGAGTCCCGCCGATTCCTTC
CTCCGATATGCCGCAACCGATTCTTGAACGAAACAACAAAGTGTATGAACAGTGCCAAGAAAGCTCAAAGCAGTTCC
CGAAATCAGCTGGGATTATCGTAAACACGTTTGAATCTCTCGAACCCAGAGCTCTCAGAGCAATATGGGACGGTCTG
TGCTTGACCGAGAACGTTCCAACTCCACCGGTCTACCCCATCGGACCGCTGATTGTTTCCCACGGCGGTGGAGGCCG
CGGGGCCGAGTGTTTGAAATGGCTGGACTCACAGCCAAGTGGAAGCGTGGTGTTCCTCTGTTTTGGGAGCTTGGGAT
TGTTTTCAAAGGAGCAGTTGAAGGAAATTGCGATTGGGTTGGAGAATAGTGGGCACAGATTTTTGTGGGTGGTCCGT
AATCCTCCAGCCCAAAATCAAATTGGGCTGGTTATTAAAGAGTCCGATCCGGAATTGAAATCTTTGCTTCCGGACGG
GTTCTTGGATCGGACTAAGGATCGGGGTCTGGTGGTCAAGTCATGGGTCCCGCAAGTGGCAGTGTTGAATCACAACT
CGGTGGGTGGGTTTGTGAGTCATTGCGGGTGGAACTCGGTGTTGGAATCGGTGTGTGCCGGTGTGCCGATTGTGTCT
TGGCCGCTCTACGCGGAGCAGAGATTAAATCGAGTGGTTTTGGTGGAGGAGATTAAGATTGCTATGCCGATGAACGA
GTCTGAAGACGGGTTTGTGAGAGCAGCGGAGGTGGAGAAGCGAGTTACGGAGTTGATGGACTCGGAGGAGGGCGAGT
CGATCAGGAAGCGTACAAAGGATTTGCAAAACGATGCCCATGCAGCATTGGGTGAGACCGGGTCGTCTGGGGTTGCA
TTTACTAAACTACTTGAATTGTGGGGCAAAACTGGTTAG
>Seq16 [organism = Malus trilobata] phloretin 4′-O-glycosyltransferase (MtbPGT2-2),
complete cds
ATGGAGGCGGCAGCTATAGTTTTATATCCATCACCAGTAATTGGCCACTTGATCGCCATGGTAGAGCTAGGCAAGCT
CATAATCACCCGCCACCCTTCTCTGTGCATCCACATCCTCATCACCACCCCGCCCTACCGCGCCAACGACACCGACT
CATACATCACCTCCGTCTCCGCTGCCAACCCTTCCCTCATCTTCCACCACCTCCCCACCATCTCCCTCCCTCCCTCC
CTCGCCTCCTCCCGCAACCACGAAACCCTAACCTTCGGACTCGCCCCCCTCAACAACCCTAACGTCCACCAAGCCCT
CCTCTCCATCTCCCACAACTTCTCCATCAAAGCTTTTGTCATGGACTTCTTCTGCTCTGTCGGGCTCCCCATTGCCA
CCGAGCTGAACATCCCCAGCTACTTCTTCTTCACATCCAGCGCCACCACCCTCGCTTCCTTCCTCTACCTCCCCACC
ATTCACAACATCACTGACAAAAGCCTCAAAGACCTAAATATCCTTCTCAACATTCCAGGAGTCCCGCCGATTCCTTC
CTCCGATATGCCGCAACCGATTCTTGAACGAAACAACAAAGTGTATGAACAGTGCCAAGAAAGCTCAAAGCAGTTCC
CGAAATCAGCTGGGATTATCGTAAACACGTTTGAATCTCTCGAACCCAGAGCTCTCAGAGCAATATGGGACGGTCTG
TGCTTGACCGAGAACGTTCCAACTCCACCGGTCTACCCCATCGGACCGCTGATTGTTTCCCACGGCGGTGGAGGCCG
CGGGGCCGAGTGTTTGAAATGGCTGGACTCACAGCCAAGTGGAAGCGTGGTGTTCCTCTGTTTTGGGAGCTTGGGAT
TGTTTTCAAAGGAGCAGTTGAAGGAAATTGCGATTGGGTTGGAGAATAGTGGGCACAGATTTTTGTGGGTGGTCCGT
AATCCTCCAGCCCAAAATCAAATTGGGCTGGTTATTAAAGAGTCCGATCCGGAATTGAAATCTTTGCTTCCGGACGG
GTTCTTGGATCGGACTAAGGATCGGGGTCTGGTGGTCAAGTCATGGGTCCCGCAAGTGGCAGTGTTGAATCACAACT
CGGTGGGTGGGTTTGTGAGTCATTGCGGGTGGAACTCGGTGTTGGAATCGGTGTGTGCCGGTGTGCCGATTGTGTCT
TGGCCGCTCTACGCGGAGCAGAGATTAAATCGAGTGGTTTTGGTGGAGGAGATTAAGATTGCTATGCCGATGAACGA
GTCTGAAGACGGGTTTGTGAGAGCAGCGGAGGTGGAGAAGCGAGTTACGGAGTTGATGGACTCGGAGGAGGGCGAGT
CGATCAGGAAGCGTACAAAGGATTTGCAAAACGATGCCCATGCAGCATTGGGTGAGACCGGGTCGTCTGGGGTTGCA
TTTACTAAACTACTTGAATTGTGGGGCAAAACTGGTTAG
>Seq17 [organism = Malus] phloretin 4′-O-glycosyltransferase (AoteaPGT2-1),
complete cds
ATGGAGGCGGCAGCTATAGTTTTATATCCATCACCGCTAATTGGGCACTTAGTCTCCATGGTAGAGCTAGGCAAGCT
CATACTCACCCGCCACCCTTCTCTGTGCATCCACATCCTCATCACCACCCCGCCCTACCGCGCCAACGACACCGACT
CATACATCACCTCCGTCTCCGCTGCCAACCCTTCCCTCATCTTCCACCACCTCCCCACCATCTCCCTCCCTCCCTCC
CTCGCCTCCTCCCGCAACCACGAAATCCTAGCCTTCGAACTCGCCCCCCTCTACAACCCTAACGTCCACCAAGCCCT
CGTCTCCATCTCCCACAACTTCTCCATCAAAGCTTTTGTCATGGACTTCTTCTGCTATGTCGGGCTCCCCGTTGCCA
CCGAGCTGAACATCCCCAGCTACTTCTTCTTCACATCCAGCGCCAACACCCTCGCTTCCTCCCTCTACCTCCCCACC
CTTCACAACATCATTGACAAAAGCCTCAAAGACCTAAATATCCTTCTCAACATTCCAGGAGTCCCGCCGATGCCTTC
CTCCGATATGCCGCAACCGACTCTTGATCGAAACCAAAAAGTGTATGAACATGTCCAAGGAAGCTCAAAGCAGTTCC
CGAAATCAGCTGGGATTATCGTAAACACGTTTGAATCTCTCGAACCCAGAGCTCTCAGAGCAATATGGGACGGTCTG
TGCTTGCCCGAGAACGTTCCAACTCCACCGGTCTACCCCATCGGACCGCTGATTGTTTCCCATGGCGGTGGAGGCCG
CGGGGCCGAGTGTTTGAAATGGCTGGACTCACAGCCAAGTGGAAGCGTGGTGTTCCTCTGTTTTGGGAGCTTGGGAT
TGTTTTCAAAGGAGCAGTTGAAGGAAATTGCGATTGGGTTGGAGAATAGTGGGCACAGATTTTTGTGGGTGGTCCGT
AATCCTCCAGCCCAAAATCAAATTGGGCTGGATATTAAAGAGTCCGATCCGGAATTGAAATCTTTGCTTCCGGACGG
GTTTTTGGATCGGACTAAGGATCGGGGTCTCGTGGTCAAGTCATGGGCCCCGCAAGCGGCAGTGTTGAATCATAACT
CGGTGGGTGGGTTTGTGAGTCATTGCGGGTGGAACTCGGTGTTGGAATCGGTGTGTGCCGGTGTGCCGATTGTGGCT
TGGCCGCTCTACGCGGAGCAGAGATTCAATCGAGTGGTTTTGGTGAAGGAGATTAAGATTGCTATGCCGATGAACGA
GTCAGAAGACGGGTTTGTGAGAGCAGCGGAGGTGGAGAAGCGAATTACGGAGTTGATGGACTCGGAGGAGGGCGCGT
CGATCAGGAAGCGTACAAAGGATTTGCAAAACAATGCCCATGCAGCATTGGGTGAGACCGGGTCGTCTGGGGTTGCA
TTGACTAAACTACTTGAATTGTGGGGCAAAACTGGTTAG
>Seq18 [organism = Malus domestica] Cazyme ID: MDP0000836043. UDP-glucosyl
transferase 88A1, complete cds
ATGGAGGCGACAGCTATAGTTTTATATCCATCACCTCTAATTGGGCACTTAGTCTCCATGGTAGAGCTAGGCAAGCT
CATACTCACCCGCCACCCTTCTCTGTGCATCCACATCCTCATCACCACCCCGCCCTACCGTGCCAACGACACCGACT
CATACATCACCTCCGTCTCCGCCGCCAACCCTTCCCTCATTTTCCACCACCTCCCCACCATCTCCCTCCCTCCCTCC
CTCTCCCCCTCCCGCAACCACGAAACCCCAATCTTCGAAGTCCTTCTCCTCAACAACCCTTACGTCCACCAAGCCCT
CCTCTCCATCTCCCACAACTTCTCCATCAAAGCTTTTGTCATGGACTTCTTCTGCTCTGTCGGGCTCCCCATTGCCA
CCGAGCTGAACATCCCCAGCTACTTCTTCTTCACATCCAGCGCCGCCAACCTCGCTTGCTTCCTCTACCTCCCCACC
ATTCACAGCATCACTGACAAAAGCCTCAAAGACCTAAATATCCTTCTCAACATTCCAGGAGTCCAGCCGATTCCTTC
CTCCGATATGCCGAAACCGATTCTTGAACGAAACAACAAAGTGTATGAACATTTCCAAGAAAGCTCAAAGCAGTTCC
CGAAATCAGCTGGGATTATCGTAAACACGTTTGAATCTCTCGAACCCAGAGTTCTCAGAGCAATATGGGACGGTCTG
TGCTTGACGGAGAACGTTCCAACTCCACCGGTCTACCCCATCGGACCGCTGATTATTTCCCATGGCGGTGGAGGCCG
CGGGGCCGAGTATTTGAAATGGCTGGACTCACAGCCAAGTGGAAGCGTGGTGTTCCTCTGTTTTGGGAGCTTGGGAT
TGTTTTCAAAGGAGCAGTTGAAGGAAATTGCGATTGGGTTGGAGAATAGTGGGCACAGATTTTTGTGGGTGGTCCGT
AATCCTCCAGCCCAAAATCAAATTGGGCTGGCTATTAAAGAGTCCGATCCGGAATTGAAATCTTTGCTTCCGGACGG
GTTCTTGGATCGGACTAAGGGTCGGGGTCTCGTGGTCAAGTCATGGGCCCCGCAAGTGGCAGTGTTGAATCACAACT
CGGTGGGTGGGTTTGTGAGTCATTGCGGGTGGAACTCGGTGTTGGAATCGGTGTGTGCCGGTGTGCCGATTGTGGCT
TGGCCGCTCTACGCGGAGCAGAGATTCAATCGAGTGGTTTTGGTGGAGGAGATTAAGATTGCTATGCCGATGAACGA
GTCAGAAGACGGGTTTGTGAGAGCAGCGGAGGTGGAGAAGCGAGTTACGGAGTTGATGGACTCGGAGGAGGGCGAGT
CGATCAGGAAGCGTACAAAGGATTTGCAAAACGATGCCCATGCAGCATTGGGTGAGACCGGGTCGTCTCGGGTTGCA
TTTACTAAACTACTTGAATTCTGGGGCAAAACCTGTTAG

### REFERENCES

Andre, C. M., Greenwood, J. M., Walker, E. G., Rassam, M., Sullivan, M., Evers, D., Perry, N. B., Laing, W. A., 2012. Anti-inflammatory procyanidins and triterpenes in 109 apple varieties. J. Agric. Food Chem. 60, 10546-10554. https://doi.org/10.1021/jf302809k
Brazier-Hicks, M., Offen, W. A., Gershater, M. C., Revett, T. J., Lim, E.-K., Bowles, D. J., Davies, G. J., Edwards, R., 2007. Characterization and engineering of the bifunctional N- and O-glucosyltransferase involved in xenobiotic metabolism in plants. Proc. Natl. Acad. Sci. U.S.A 104, 20238-20243. https://doi.org/10.1073/pnas.0706421104
Caputi, L., Malnoy, M., Goremykin, V., Nikiforova, S., Martens, S., 2012. A genome-wide phylogenetic reconstruction of family 1 UDP-glycosyltransferases revealed the expansion of the family during the adaptation of plants to life on land. Plant J. Cell Mol. Biol. 69, 1030-1042. https://doi.org/10.1111/j.1365-313X.2011.04853.x
Chagné, D., Crowhurst, R. N., Troggio, M., Davey, M. W., Gilmore, B., Lawley, C., Vanderzande, S., Hellens, R. P., Kumar, S., Cestaro, A., Velasco, R., Main, D., Rees, J. D., Iezzoni, A., Mockler, T., Wilhelm, L., Van de Weg, E., Gardiner, S. E., Bassil, N., Peace, C., 2012. Genome-wide SNP detection, validation, and development of an 8K SNP array for apple. PloS One 7, e31745. https://doi.org/10.1371/journal.pone.0031745
Daccord, N., Celton, J.-M., Linsmith, G., Becker, C., Choisne, N., Schijlen, E., van de Geest, H., Bianco, L., Micheletti, D., Velasco, R., Di Pierro, E. A., Gouzy, J., Rees, D. J. G., Guérif, P., Muranty, H., Durel, C.-E., Laurens, F., Lespinasse, Y., Gaillard, S., Aubourg, S., Quesneville, H., Weigel, D., van de Weg, E., Troggio, M., Bucher, E., 2017. High-quality de novo assembly of the apple genome and methylome dynamics of early fruit development. Nat. Genet. 49, 1099-1106. https://doi.org/10.1038/ng.3886
Dai, H., Li, W., Han, G., Yang, Y., Ma, Y., Li, H., Zhang, Z., 2013. Development of a seedling clone with high regeneration capacity and susceptibility to Agrobacterium in apple. Sci. Hortic. 164, 202-208. https://doi.org/10.1016/j.scienta.2013.09.033
Dare, A. P., Tomes, S., Jones, M., McGhie, T. K., Stevenson, D. E., Johnson, R. A., Greenwood, D. R., Hellens, R. P., 2013. Phenotypic changes associated with RNA interference silencing of chalcone synthase in apple (Malus x domestica). Plant J. Cell Mol. Biol. 74, 398-410. https://doi.org/10.1111/tpj.12140
Dare, A. P., Yauk, Y.-K., Tomes, S., McGhie, T. K., Rebstock, R. S., Cooney, J. M., Atkinson, R. G., 2017. Silencing a phloretin-specific glycosyltransferase perturbs both general phenylpropanoid biosynthesis and plant development. Plant J. Cell Mol. Biol. 91, 237-250. https://doi.org/10.1111/tpj.13559
Eichenberger, M., Lehka, B. J., Folly, C., Fischer, D., Martens, S., Simón, E., Naesby, M., 2017. Metabolic engineering of Saccharomyces cerevisiae for de novo production of dihydrochalcones with known antioxidant, antidiabetic, and sweet tasting properties. Metab. Eng. 39, 80-89. https://doi.org/10.1016/j.ymben.2016.10.019
Elejalde-Palmett, C., Billet, K., Lanoue, A., De Craene, J.-O., Glévarec, G., Pichon, O., Clastre, M., Courdavault, V., St-Pierre, B., Giglioli-Guivarc'h, N., Dugé de Bernonville, T., Besseau, S., 2019. Genome-wide identification and biochemical characterization of the UGT88F subfamily in Malus x domestica Borkh. Phytochemistry 157, 135-144. https://doi.org/10.1016/j.phytochem.2018.10.019
Espley, R. V., Hellens, R. P., Putterill, J., Stevenson, D. E., Kutty-Amma, S., Allan, A. C., 2007. Red colouration in apple fruit is due to the activity of the MYB transcription factor, MdMYB10. Plant J. 49, 414-427. https://doi.org/10.1111/j.1365-313X.2006.02964.x
Fan, X., Zhang, Y., Dong, H., Wang, B., Ji, H., Liu, X., 2015. Trilobatin attenuates the LPS-mediated inflammatory response by suppressing the NF-KB signaling pathway. Food Chem. 166, 609-615. https://doi.org/10.1016/j.foodchem.2014.06.022
Fukuchi-Mizutani, M., Okuhara, H., Fukui, Y., Nakao, M., Katsumoto, Y., Yonekura-Sakakibara, K., Kusumi, T., Hase, T., Tanaka, Y., 2003. Biochemical and molecular characterization of a novel UDP-glucose:anthocyanin 3′-O-glucosyltransferase, a key enzyme for blue anthocyanin biosynthesis, from gentian. Plant Physiol. 132, 1652-1663. https://doi.org/10.1104/pp. 102.018242
Gao, L., Li, Z., Xia, C., Qu, Y., Liu, M., Yang, P., Yu, L., Song, X., 2017. Combining manipulation of transcription factors and overexpression of the target genes to enhance lignocellulolytic enzyme production in Penicillium oxalicum. Biotechnol. Biofuels 10, 100. https://doi.org/10.1186/s13068-017-0783-3
Gietz, R. D. and Schiestl, R. H. (2007) Quick and easy yeast transformation using the LiAc/SS carrier DNA/PEG method. Nat. Protoc., 2, 35-37.
Gosch, C., Flachowsky, H., Halbwirth, H., Thill, J., Mjka-Wittmann, R., Treutter, D., Richter, K., Hanke, M.-V., Stich, K., 2012. Substrate specificity and contribution of the glycosyltransferase UGT71A15 to phloridzin biosynthesis. Trees 26, 259-271. https://doi.org/10.1007/s00468-011-0669-0
Gosch, C., Halbwirth, H., Kuhn, J., Miosic, S., Stich, K., 2009. Biosynthesis of phloridzin in apple (Malus domestica Borkh.). Plant Sci. 176, 223-231. https://doi.org/10.1016/j.plantsci.2008.10.011
Gosch, C., Halbwirth, H., Schneider, B., Hölscher, D., Stich, K., 2010. Cloning and heterologous expression of glycosyltransferases from Malus x domestica and Pyrus communis, which convert phloretin to phloretin 2′-O-glucoside (phloridzin). Plant Sci. 178, 299-306. https://doi.org/10.1016/j.plantsci.2009.12.009
Gutierrez, B. L., Arro, J., Zhong, G.-Y., Brown, S. K., 2018a. Linkage and association analysis of dihydrochalcones phloridzin, sieboldin, and trilobatin in Malus. Tree Genet. Genomes 14, 91. https://doi.org/10.1007/si1295-018-1304-7
Gutierrez, B. L., Zhong, G.-Y., Brown, S. K., 2018b. Genetic diversity of dihydrochalcone content in Malus germplasm. Genet. Resour. Crop Evol. 65, 1485-1502. https://doi.org/10.1007/s10722-018-0632-7
Hellens, R. P., Allan, A. C., Friel, E. N., Bolitho, K., Grafton, K., Templeton, M. D., Karunairetnam, S., Gleave, A. P., Laing, W. A., 2005. Transient expression vectors for functional genomics, quantification of promoter activity and RNA silencing in plants. Plant Methods 1, 13. https://doi.org/10.1186/1746-4811-1-13
Hsu, Y.-H., Tagami, T., Matsunaga, K., Okuyama, M., Suzuki, T., Noda, N., Suzuki, M., Shimura, H., 2017. Functional characterization of UDP-rhamnose-dependent rhamnosyltransferase involved in anthocyanin modification, a key enzyme determining blue coloration in Lobelia erinus. Plant J. Cell Mol. Biol. 89, 325-337. https://doi.org/10.1111/tpj.13387
Ibdah, M., Berim, A., Martens, S., Valderrama, A. L. H., Palmieri, L., Lewinsohn, E., Gang, D. R., 2014. Identification and cloning of an NADPH-dependent hydroxycinnamoyl-CoA double bond reductase involved in dihydrochalcone formation in Malusxdomestica Borkh. Phytochemistry 107, 24-31. https://doi.org/10.1016/j.phytochem.2014.07.027
Ishii, J., Kondo, T., Makino, H., Ogura, A., Matsuda, F. and Kondo, A. (2014) Three gene expression vector sets for concurrently expressing multiple genes in Saccharomyces cerevisiae. FEMS Yeast Res., 14, 399-411.
Jia, Z., Yang, X., Hansen, C. A., Naman, C. B., Simons, C. T., Slack, J. P., Gray, K., 2008. Consumables. WO2008148239A1.
Jugdé, H., Nguy, D., Moller, I., Cooney, J. M., Atkinson, R. G., 2008. Isolation and characterization of a novel glycosyltransferase that converts phloretin to phlorizin, a potent antioxidant in apple. FEBS J. 275, 3804-3814. https://doi.org/10.1111/j.1742-4658.2008.06526.x
Lei, L., Huang, B., Liu, A., Lu, Y.-J., Zhou, J.-L., Zhang, J., Wong, W.-L., 2018. Enzymatic production of natural sweetener trilobatin from citrus flavanone naringin using immobilised α-I-rhamnosidase as the catalyst. Int. J. Food Sci. Technol. 53, 2097-2103. https://doi.org/10.1111/ijfs.13796
Li, Y., Baldauf, S., Lim, E. K., Bowles, D. J., 2001. Phylogenetic analysis of the UDP-glycosyltransferase multigene family of Arabidopsis thaliana. J. Biol. Chem. 276, 4338-4343. https://doi.org/10.1074/jbc.M007447200
Livak, K. J., Schmittgen, T. D., 2001. Analysis of relative gene expression data using real-time quantitative PCR and the 2(-Delta Delta C(T)) Method. Methods San Diego Calif. 25, 402-408. https://doi.org/10.1006/meth.2001.1262
Malnoy, M., Reynoird, J. P., Mourgues, F., Chevreau, E., Simoneau, P., 2001. A method for isolating total RNA from pear leaves. Plant Mol. Biol. Report. 19, 69-69. https://doi.org/10.1007/BF02824081
Nieuwenhuizen, N. J., Green, S. A., Chen, X., Bailleul, E. J. D., Matich, A. J., Wang, M. Y., Atkinson, R. G., 2013. Functional genomics reveals that a compact terpene synthase gene family can account for terpene volatile production in apple. Plant Physiol. 161, 787-804. https://doi.org/10.1104/pp. 112.208249
Ross, J., Li, Y., Lim, E., Bowles, D. J., 2001. Higher plant glycosyltransferases. Genome Biol. 2, REVIEWS3004. https://doi.org/10.1186/gb-2001-2-2-reviews3004
Schrödinger, LLC, 2015. The PyMOL Molecular Graphics System, Version 2.0.
Sun, X., Wang, P., Jia, X., Huo, L., Che, R., Ma, F., 2018. Improvement of drought tolerance by overexpressing MdATG18a is mediated by modified antioxidant system and activated autophagy in transgenic apple. Plant Biotechnol. J. 16, 545-557. https://doi.org/10.1111/pbi.12794
Sun, Y., Li, W., Liu, Z., 2015. Preparative isolation, quantification and antioxidant activity of dihydrochalcones from Sweet Tea (Lithocarpus polystachyus Rehd.). J. Chromatogr. B Analyt. Technol. Biomed. Life. Sci. 1002, 372-378. https://doi.org/10.1016/j.jchromb.2015.08.045
Sun, Y.-S., Zhang, Y., 2017. A method of sweetening natural bulk separation trilobatin. CN104974201B.
Tanaka, T., Tanaka, O., Kohda, H. (Hiroshima U. (Japan) F. of M., Chou, W. H., Chen, F. H., 1983. Isolation of trilobatin, a sweet dihydrochalcone-glucoside from leaves of Vitis piasezkii Maxim. and V. saccharifera Makino. Agric. Biol. Chem. Jpn.
van Ooijen, J. W., Van Ooijen, J. W., van Ooijen, J. W., Van Ooijen, J. W., Van Ooijen, J., van 't Verlaat, J. W., Ooijen, J. W., van Tol, J., Dalen, J., Ooijen, J. W. van, Buren, J., van der Meer, J., Van Krieken, J. H., Ooijen, J. W. V., van Kessel, J., Van, O., Voorrips, R., van den Heuvel, L. P. W. J., 2006. JoinMap® 4. Software for the calculation of genetic linkage maps in experimental populations.
Velasco, R., Zharkikh, A., Affourtit, J., Dhingra, A., Cestaro, A., Kalyanaraman, A., Fontana, P., Bhatnagar, S. K., Troggio, M., Pruss, D., Salvi, S., Pindo, M., Baldi, P., Castelletti, S., Cavaiuolo, M., Coppola, G., Costa, F., Cova, V., Dal Ri, A., Goremykin, V., Komjanc, M., Longhi, S., Magnago, P., Malacarne, G., Malnoy, M., Micheletti, D., Moretto, M., Perazzolli, M., Si-Ammour, A., Vezzulli, S., Zini, E., Eldredge, G., Fitzgerald, L. M., Gutin, N., Lanchbury, J., Macalma, T., Mitchell, J. T., Reid, J., Wardell, B., Kodira, C., Chen, Z., Desany, B., Niazi, F., Palmer, M., Koepke, T., Jiwan, D., Schaeffer, S., Krishnan, V., Wu, C., Chu, V. T., King, S. T., Vick, J., Tao, Q., Mraz, A., Stormo, A., Stormo, K., Bogden, R., Ederle, D., Stella, A., Vecchietti, A., Kater, M. M., Masiero, S., Lasserre, P., Lespinasse, Y., Allan, A. C., Bus, V., Chagné, D., Crowhurst, R. N., Gleave, A. P., Lavezzo, E., Fawcett, J. A., Proost, S., Rouzé, P., Sterck, L., Toppo, S., Lazzari, B., Hellens, R. P., Durel, C.-E., Gutin, A., Bumgarner, R. E., Gardiner, S. E., Skolnick, M., Egholm, M., Van de Peer, Y., Salamini, F., Viola, R., 2010. The genome of the domesticated apple (Malus x domestica Borkh.). Nat. Genet. 42, 833-839. https://doi.org/10.1038/ng.654
Voinnet, O., Rivas, S., Mestre, P., Baulcombe, D., 2003. An enhanced transient expression system in plants based on suppression of gene silencing by the p19 protein of tomato bushy stunt virus. Plant J. Cell Mol. Biol. 33, 949-956.
Vrhovsek, U., Masuero, D., Gasperotti, M., Franceschi, P., Caputi, L., Viola, R. and Mattivi, F. (2012) A versatile targeted metabolomics method for the rapid quantification of multiple classes of phenolics in fruits and beverages. J. Agric. Food Chem., 60, 8831-8840.
WALTON, S. K., DENARDO, T. N. M. I., ZANNO, P. R., TOPALOVIC, M. N. M. I., 2013. Taste modifiers. WO2013074811A1.
Wang, Y., Yauk, Y.-K., Zhao, Q., et al. (2020) Biosynthesis of the dihydrochalcone sweetener trilobatin requires Phloretin Glycosyltransferase 2. Plant Physiol., 184, 738-752.
Williams, A. H., 1982. Chemical evidence from the flavonoids relevant to the classification of Malus species. Bot. J. Linn. Soc. 84, 31-39. https://doi.org/10.1111/j.1095-8339.1982.tb00358.x
Xiao, Z., Zhang, Y., Chen, X., Wang, Y., Chen, W., Xu, Q., Li, P., Ma, F., 2017. Extraction, identification, and antioxidant and anticancer tests of seven dihydrochalcones from Malus “Red Splendor” fruit. Food Chem. 231, 324-331. https://doi.org/10.1016/j.foodchem.2017.03.111
Yahyaa, M., Ali, S., Davidovich-Rikanati, R., Ibdah, Muhammad, Shachtier, A., Eyal, Y., Lewinsohn, E., Ibdah, Mwafaq, 2017. Characterization of three chalcone synthase-like genes from apple (Malus x domestica Borkh.). Phytochemistry 140, 125-133. https://doi.org/10.1016/j.phytochem.2017.04.022
Yahyaa, M., Davidovich-Rikanati, R., Eyal, Y., Sheachter, A., Marzouk, S., Lewinsohn, E., Ibdah, M., 2016. Identification and characterization of UDP-glucose:Phloretin 4′-O-glycosyltransferase from Malus x domestica Borkh. Phytochemistry 130, 47-55. https://doi.org/10.1016/j.phytochem.2016.06.004
Yang, J., Zhang, Y., 2015. I-TASSER server: new development for protein structure and function predictions. Nucleic Acids Res. 43, W174-W181. https://doi.org/10.1093/nar/gkv342
Yao, J.-L., Cohen, D., Atkinson, R., Richardson, K., Morris, B., 1995. Regeneration of transgenic plants from the commercial apple cultivar Royal Gala. Plant Cell Rep. 14, 407-412. https://doi.org/10.1007/BF00234044
Yao, J.-L., Tomes, S., Gleave, A. P., 2013. Transformation of apple (Malus x domestica) using mutants of apple acetolactate synthase as a selectable marker and analysis of the T-DNA integration sites. Plant Cell Rep. 32, 703-714. https://doi.org/10.1007/s00299-013-1404-7
Yauk, Y.-K., Ged, C., Wang, M. Y., Matich, A. J., Tessarotto, L., Cooney, J. M., Chervin, C., Atkinson, R. G., 2014. Manipulation of flavour and aroma compound sequestration and release using a glycosyltransferase with specificity for terpene alcohols. Plant J. Cell Mol. Biol. 80, 317-330. https://doi.org/10.1111/tpj.12634
Zhou, K., Hu, L., Li, Y., Chen, X., Zhang, Z., Liu, B., Li, P., Gong, X., Ma, F., 2019. MdUGT88F1-Mediated Phloridzin Biosynthesis Regulates Apple Development and Valsa Canker Resistance. Plant Physiol. 180, 2290-2305. https://doi.org/10.1104/pp. 19.00494

1. A method of producing a plant cell or plant with increased trilobatin content, the method comprising transformation of a plant cell with a polynucleotide encoding a polypeptide with the amino acid sequence of any one of SEQ ID NO: 1 to 9, or a variant of the polypeptide.

2. A method of producing a plant cell or plant with increased 4′-O-glycosyltransferase activity, the method comprising transformation of a plant cell with a polynucleotide encoding a polypeptide with the amino acid sequence of any one of SEQ ID NO: 1 to 9, or a variant of the polypeptide.

3. The method of claim 1 or 2, wherein the variant has 4′-O-glycosyltransferase activity.

4. The method of any one of claims 1 to 3, wherein the variant has at least 70% sequence identity to a polypeptide with the amino acid sequence of any one of SEQ ID NO: 1 to 9.

5. The method of any one of claims 1 to 3, wherein the polynucleotide encodes a polypeptide with an amino acid sequence that has at least 85% identity to the sequence of SEQ ID NO: 1.

6. The method of any one of claim 1 to 3, wherein the polynucleotide encodes a polypeptide with the amino acid sequence of SEQ ID NO: 1.

7. The method of any one of claims 1 to 6, wherein the plant cell or plant is also transformed with a polynucleotide encoding a chalcone synthase (CHS), or a chalcone synthase (CHS) and a double bond reductase (DBR).

8. A method of producing a plant cell or plant with increased trilobatin content, the method comprising transformation of a plant cell with a polynucleotide comprising a nucleotide sequence selected from any one of the sequences SEQ ID NO: 10 to 18, or a variant thereof.

9. A method of producing a plant cell or plant with increased 4′-O-glycosyltransferase activity, the method comprising transformation of a plant cell with a polynucleotide comprising a nucleotide sequence selected from any one of the sequences SEQ ID NO: 10 to 18, or a variant thereof.

10. The method of claim 8 or 9, wherein the variant encodes a polypeptide that has 4′-O-glycosyltransferase activity.

11. The method of any one of claims 8 to 10, wherein the variant comprises a sequence that has at least 70% sequence identity to the nucleotide sequence of any one of SEQ ID NO: 10 to 18.

12. The method of any one of claims 8 to 10, wherein the variant comprises a sequence that has at least 85% sequence identity to the nucleotide sequence of SEQ ID NO: 10.

13. The method of any one of claims 8 to 10, wherein the polynucleotide comprises the sequence of SEQ ID NO: 10.

14. The method of any one of claims 8 to 13, wherein the plant cell or plant is also transformed with a polynucleotide encoding a chalcone synthase (CHS), or a chalcone synthase (CHS) and a double bond reductase (DBR).

15. A method of producing a plant cell or plant with increased trilobatin content or increased 4′-O-glycosyltransferase activity, the method comprising upregulating in the plant cell or plant expression of a polypeptide with the amino acid sequence of any one of SEQ ID NO: 1 to 9, or a variant of the polypeptide.

16. A method of producing a plant cell or plant with increased trilobatin content or increased 4′-O-glycosyltransferase activity, the method comprising upregulating in the plant cell or plant expression of a polynucleotide comprising a nucleotide sequence selected from any one of the sequences SEQ ID NO: 10 to 18, or a variant thereof.

17. The method of claim 15 or 16, wherein the upregulating comprises genetic engineering.

18. The method of claim 15 or 16, wherein the upregulating comprises crossing with a plant which expresses a polypeptide comprising an amino acid sequence having at least 70% sequence identity to a polypeptide with the amino acid sequence of any one of SEQ ID NO: 1 to 9.

19. The method of claim 15 or 16, wherein the upregulating comprises crossing with a plant which expresses a polynucleotide comprising a nucleotide sequence selected from any one of the sequences SEQ ID NO: 10 to 18.

20. The method of any one of claims 15 to 19, wherein the plant cell or plant comprises or is also transformed with a polynucleotide encoding a chalcone synthase (CHS), or a chalcone synthase (CHS) and a double bond reductase (DBR).

21. A genetic construct comprising a polynucleotide encoding a polypeptide with the amino acid sequence of any one of SEQ ID NO: 1 to 9 or a variant of the polypeptide.

22. A genetic construct comprising a polynucleotide comprising a nucleotide sequence selected from any one of the sequences SEQ ID NO: 10 to 18 or a variant thereof.

23. The genetic construct of claim 21 or 22, wherein the genetic construct further comprises a polynucleotide encoding a chalcone synthase (CHS) and/or a double bond reductase (DBR).

24. A host cell comprising the genetic construct of any one of claims 21 to 23.

25. A host cell genetically modified to express a polynucleotide encoding a polypeptide with the amino acid sequence of any one of SEQ ID NO: 1 to 9 or a variant of the polypeptide.

26. A host cell genetically modified to express a polynucleotide comprising a nucleotide sequence selected from any one of the sequences SEQ ID NO: 10 to 18 or a variant thereof.

27. The host cell of any one of claims 24 to 26, wherein the host cell is a bacterial, fungal or yeast cell, an insect cell, a plant cell, or a mammalian cell.

28. A method for the biosynthesis of trilobatin comprising the steps of culturing the host cell of any one of claims 24 to 27, capable of expressing a 4′-O-glycosyltransferase, in the presence of phloretin which may be supplied to, or may be naturally present in the host cell.

29. A method of producing trilobatin, the method comprising extracting trilobatin from the host cell of any one of claims 24 to 27.

30. A plant cell genetically modified to express a polynucleotide encoding a polypeptide with the amino acid sequence of any one of SEQ ID NO: 1 to 9 or a variant of the polypeptide.

31. A plant cell genetically modified to express a polynucleotide comprising a nucleotide sequence selected from any one of the sequences SEQ ID NO: 10 to 18 or a variant thereof.

32. A plant comprising the plant cell of claim 30 or 31.

33. A method for selecting a plant with altered 4′-O-glycosyltransferase activity, the method comprising testing a plant for altered expression of a polynucleotide encoding a polypeptide with the amino acid sequence of any one of SEQ ID NO: 1 to 9 or a variant of the polypeptide.

34. A method for selecting a plant with altered 4′-O-glycosyltransferase activity, the method comprising testing a plant for altered expression of a polynucleotide comprising a nucleotide sequence selected from any one of the sequences SEQ ID NO: 10 to 18 or a variant thereof.

35. A method for selecting a plant with altered trilobatin content, the method comprising testing a plant for altered expression of a polynucleotide encoding a polypeptide with the amino acid sequence of any one of SEQ ID NO: 1 to 9 or a variant of the polypeptide.

36. A method for selecting a plant with altered trilobatin content, the method comprising testing a plant for altered expression of a polynucleotide comprising a nucleotide sequence selected from any one of the sequences SEQ ID NO: 10 to 18 or a variant thereof.

37. A plant cell or plant produced by the method of any one of claims 1 to 20.

38. A plant cell or plant selected by the method of any one of claims 33 to 36.

39. A group or population of plants produced by the method of any one of claims 1 to 20.

40. A method of producing trilobatin, the method comprising extracting trilobatin from the plant cell or plant of any one of claims 30, 31, 32, 37 or 38.

41. A method of producing trilobatin, the method comprising contacting phloretin with UDP-glucose and the expression product of an expression construct encoding a polypeptide with the amino acid sequence of any one of SEQ ID: NO 1 to 9 or a variant of the polypeptide, or a polynucleotide comprising a nucleotide sequence selected from any one of the sequences SEQ ID NO: 10 to 18 or a variant thereof, to obtain trilobatin.


SEQUENCE LISTING (18 sequences)

SEQ ID NO: 1 - protein, 474 aa, Malus toringoides
MEATAIVLYPSPLIGHLVSMVELGKLILTRHPSLCIHILITTPPYRANDTDSYITSVSAANPSLIFHHLPTISLPPSLASSRNHEILAFELAPLYNPNVHQALVSISHNFSIKAFVMDFFCYVGLPVATELNIPSYFFFTSSANTLASSLYLPTLHNIIDKSLKDLNILLNIPGVPPMPSSDMPQPTLDRNQKVYEHVQGSSKQFPKSAGIIVNTFESLEPRALRAIWDGLCLPENVPTPPVYPIGPLIVSHGGGGRGAECLKWLDSQPSGSVVFLCFGSLGLFSKEQLKEIAIGLENSGHRFLWVVRNPPAQNQIGLDIKESDPELKSLLPDGFLDRTKDRGLVVKSWAPQAAVLNHNSVGGFVSHCGWNSVLESVCAGVPIVAWPLYAEQRFNRVVLVKEIKIAMPMNESEDGFVRAAEVEKRITELMDSEEGASIRKRTKDLQNNAHAALGETGSSGVALTKLLELWGKTC

SEQ ID NO: 2 - protein, 474 aa, Malus toringoides
MEATAIVLYPSPLIGHLVSMVELGKLILTRHPSLCIHILITTPPYRANDTDSYITSVSAANPSLIFHHLPTISLPPSLASSRNHEILAFELAPLYNPNVHQALVSISHNFSIKAFVMDFFCYVGLPVATELNIPSYFFFTSSANTLASSLYLPTLHNIIDKSLKDLNILLNIPGVPPMPSSDMPQPTLDRNQKVYEHVQGSSKQFPKSAGIIVNTFESLEPRALRAIWDGLCLPENVPTPPVYPIGPLIVSHGGGGRGAECLKWLDSQPSGSVVFLCFGSLGLFSKEQLKEIAIGLENSGHRFLWVVRNPPAQNQIGLDIKESDPELKSLLPDGFLDRTKDRGLVVKSWAPQAAVLNHNSVGGFVSHCGWNSVLESVCAGVPIVAWPLYAEQRFNRVVLVKEIKIAMPMNESEDGFVRAAEVEKRITELMDSEEGASIRKRTKDLQNNAHAALGETGSSGVALTKLLEFWGKTC

SEQ ID NO: 3 - protein, 474 aa, Malus sieboldii; identical to SEQ ID NO: 2

SEQ ID NO: 4 - protein, 474 aa, Malus ‘Adams’; identical to SEQ ID NO: 2

SEQ ID NO: 5 - protein, 474 aa, Malus domestica; identical to SEQ ID NO: 2

SEQ ID NO: 6 - protein, 474 aa, Malus trilobata
MEAAAIVLYPSPPIGHLVSMVELGKLILTRHPSLCIHILITTPPYRANDTDSYITSVSAANPSLIFHHLPTISLPPSLASSRNHETLTFGLAPLNNPNVHQALLSISHNFSIKAFVMDFFCSVGLPIATELNIPSYFFFTSSATTLASFLYLPTIHNITDKSLKDLNILLNIPGVPPIPSSDMPQPILERNNKVYEQCQESSKQFPKSAGIIVNTFESLEPRALRAIWDGLCLTENVPTPPVYPIGPLIVSHGGGGRGAECLKWLDSQPSGSVVFLCFGSLGLFSKEQLKEIAIGLENSGHRFLWVVRNPPAQNQIGLVIKESDPELKSLLPDGFLDRTKDRGLVVKSWVPQVAVLNHNSVGGFVSHCGWNSVLESVCAGVPIVSWPLYAEQRLNRVVLVEEIKIAMPMNESEDGFVRAAEVEKRVTELMDSEEGESIRKRTKDLQNDAHAALGETGSSGVAFTKLLELWGKTG

SEQ ID NO: 7 - protein, 474 aa, Malus trilobata
MEAAAIVLYPSPVIGHLIAMVELGKLIITRHPSLCIHILITTPPYRANDTDSYITSVSAANPSLIFHHLPTISLPPSLASSRNHETLTFGLAPLNNPNVHQALLSISHNFSIKAFVMDFFCSVGLPIATELNIPSYFFFTSSATTLASFLYLPTIHNITDKSLKDLNILLNIPGVPPIPSSDMPQPILERNNKVYEQCQESSKQFPKSAGIIVNTFESLEPRALRAIWDGLCLTENVPTPPVYPIGPLIVSHGGGGRGAECLKWLDSQPSGSVVFLCFGSLGLFSKEQLKEIAIGLENSGHRFLWVVRNPPAQNQIGLVIKESDPELKSLLPDGFLDRTKDRGLVVKSWVPQVAVLNHNSVGGFVSHCGWNSVLESVCAGVPIVSWPLYAEQRLNRVVLVEEIKIAMPMNESEDGFVRAAEVEKRVTELMDSEEGESIRKRTKDLQNDAHAALGETGSSGVAFTKLLELWGKTG

SEQ ID NO: 8 - protein, 474 aa, Malus ‘Aotea’
MEAAAIVLYPSPLIGHLVSMVELGKLILTRHPSLCIHILITTPPYRANDTDSYITSVSAANPSLIFHHLPTISLPPSLASSRNHEILAFELAPLYNPNVHQALVSISHNFSIKAFVMDFFCYVGLPVATELNIPSYFFFTSSANTLASSLYLPTLHNIIDKSLKDLNILLNIPGVPPMPSSDMPQPTLDRNQKVYEHVQGSSKQFPKSAGIIVNTFESLEPRALRAIWDGLCLPENVPTPPVYPIGPLIVSHGGGGRGAECLKWLDSQPSGSVVFLCFGSLGLFSKEQLKEIAIGLENSGHRFLWVVRNPPAQNQIGLDIKESDPELKSLLPDGFLDRTKDRGLVVKSWAPQAAVLNHNSVGGFVSHCGWNSVLESVCAGVPIVAWPLYAEQRFNRVVLVKEIKIAMPMNESEDGFVRAAEVEKRITELMDSEEGASIRKRTKDLQNNAHAALGETGSSGVALTKLLELWGKTG

SEQ ID NO: 9 - protein, 474 aa, Malus domestica
MEATAIVLYPSPLIGHLVSMVELGKLILTRHPSLCIHILITTPPYRANDTDSYITSVSAANPSLIFHHLPTISLPPSLSPSRNHETPIFEVLLLNNPYVHQALLSISHNFSIKAFVMDFFCSVGLPIATELNIPSYFFFTSSAANLACFLYLPTIHSITDKSLKDLNILLNIPGVQPIPSSDMPKPILERNNKVYEHFQESSKQFPKSAGIIVNTFESLEPRVLRAIWDGLCLTENVPTPPVYPIGPLIISHGGGGRGAEYLKWLDSQPSGSVVFLCFGSLGLFSKEQLKEIAIGLENSGHRFLWVVRNPPAQNQIGLAIKESDPELKSLLPDGFLDRTKGRGLVVKSWAPQVAVLNHNSVGGFVSHCGWNSVLESVCAGVPIVAWPLYAEQRFNRVVLVEEIKIAMPMNESEDGFVRAAEVEKRVTELMDSEEGESIRKRTKDLQNDAHAALGETGSSRVAFTKLLEFWGKTC

SEQ ID NO: 10 - DNA, 1425 bp, Malus toringoides
  ref <308>: DATABASE ACCESSION NUMBER: GenBank MN380999
  ref <309>: DATABASE ENTRY DATE: 2020-08-07
  ref <313>: RELEVANT RESIDUES IN SEQ ID NO: (1)..(1425)
atggaggcgacagctatagttttatatccatcaccgctaattgggcacttagtctccatggtagagctaggcaagctcatactcacccgccacccttctctgtgcatccacatcctcatcaccaccccgccctaccgcgccaacgacaccgactcatacatcacctccgtctccgctgccaacccttccctcatcttccaccacctccccaccatctccctccctccctccctcgcctcctcccgcaaccacgaaatcctagccttcgaactcgcccccctctacaaccctaacgtccaccaagccctcgtctccatctcccacaacttctccatcaaagcttttgtcatggacttcttctgctatgtcgggctccccgttgccaccgagctgaacatccccagctacttcttcttcacatccagcgccaacaccctcgcttcctccctctacctccccacccttcacaacatcattgacaaaagcctcaaagacctaaatatccttctcaacattccaggagtcccgccgatgccttcctccgatatgccgcaaccgactcttgaccgaaaccaaaaagtgtatgaacatgtccaaggaagctcaaagcagttcccgaaatcagctgggattatcgtaaacacgtttgaatctctcgaacccagagctctcagagcaatatgggacggtctgtgcttgcccgagaacgttccaactccaccggtctaccccatcggaccgctgattgtttcccatggcggtggaggccgcggggccgagtgtttgaaatggctggactcacagccaagtggaagcgtggtgttcctctgttttgggagcttgggattgttttcaaaggagcagttgaaggaaattgcgattgggttggagaatagtgggcacagatttttgtgggtggtccgtaatcctccagcccaaaatcaaattgggctggatattaaagagtccgatccggaattgaaatctttgcttccggacgggtttttggatcggactaaggatcggggtctcgtggtcaagtcatgggccccgcaagcggcagtgttgaatcataactcggtgggtgggtttgtgagtcattgcgggtggaactcggtgttggaatcggtgtgtgccggtgtgccgattgtggcttggccgctctacgcggagcagagattcaatcgagtggttttggtgaaggagattaagattgctatgccgatgaacgagtcagaagacgggtttgtgagagcagcggaggtggagaagcgaattacggagttgatggactcggaggagggcgcgtcgatcaggaagcgtacaaaggatttgcaaaacaatgcccatgcagcattgggtgagaccgggtcgtctggggttgcattgactaaactacttgaattgtggggcaaaacctgttag

SEQ ID NO: 11 - DNA, 1425 bp, Malus toringoides
  ref <308>: DATABASE ACCESSION NUMBER: GenBank MN381000
  ref <309>: DATABASE ENTRY DATE: 2020-08-07
  ref <313>: RELEVANT RESIDUES IN SEQ ID NO: (1)..(1425)
atggaggcgacagctatagttttatatccatcaccgctaattgggcacttagtctccatggtagagctaggcaagctcatactcacccgccacccttctctgtgcatccacatcctcatcaccaccccgccctaccgcgccaacgacaccgactcatacatcacctccgtctccgctgccaacccttccctcatcttccaccacctccccaccatctccctccctccctccctcgcctcctcccgcaaccacgaaatcctagccttcgaactcgcccccctctacaaccctaacgtccaccaagccctcgtctccatctcccacaacttctccatcaaagcttttgtcatggacttcttctgctatgtcgggctccccgttgccaccgagctgaacatccccagctacttcttcttcacatccagcgccaacaccctcgcttcctccctctacctccccacccttcacaacatcattgacaaaagcctcaaagacctaaatatccttctcaacattccaggagtcccgccgatgccttcctccgatatgccgcaaccgactcttgaccgaaaccaaaaagtgtatgaacatgtccaaggaagctcaaagcagttcccgaaatcagctgggattatcgtaaacacgtttgaatctctcgaacccagagctctcagagcaatatgggacggtctgtgcttgcccgagaacgttccaactccaccggtctaccccatcggaccgctgattgtttcccatggcggtggaggccgcggggccgagtgtttgaaatggctggactcacagccaagtggaagcgtggtgttcctctgttttgggagcttgggattgttttcaaaggagcagttgaaggaaattgcgattgggttggagaatagtgggcacagatttttgtgggtggtccgtaatcctccagcccaaaatcaaattgggctggatattaaagagtccgatccggaattgaaatctttgcttccggacgggttcttggatcggactaaggatcggggtctcgtggtcaagtcatgggccccgcaagcggcagtgttgaatcacaactcggtgggtgggtttgtgagtcattgcgggtggaactcggtgttggaatcggtgtgtgccggtgtgccgattgtggcttggccgctctacgcggagcagagattcaatcgagtggttttggtgaaggagattaagattgctatgccgatgaacgagtcagaagacgggtttgtgagagcagcggaggtggagaagcgaattacggagttgatggactcggaggagggcgcgtcgatcaggaagcgtacaaaggatttgcaaaacaatgcccatgcagcattgggtgagaccgggtcgtctggggttgcattgactaaactacttgaattctggggcaaaacctgttag

SEQ ID NO: 12 - DNA, 1425 bp, Malus sieboldii; identical to SEQ ID NO: 11
  ref <308>: DATABASE ACCESSION NUMBER: GenBank MN381001
  ref <309>: DATABASE ENTRY DATE: 2020-08-07
  ref <313>: RELEVANT RESIDUES IN SEQ ID NO: (1)..(1425)

SEQ ID NO: 13 - DNA, 1425 bp, Malus ‘Adams’
  ref <308>: DATABASE ACCESSION NUMBER: GenBank MN381002
  ref <309>: DATABASE ENTRY DATE: 2020-08-07
  ref <313>: RELEVANT RESIDUES IN SEQ ID NO: (1)..(1425)
atggaggcgacagctatagttttatatccatcaccgctaattgggcacttagtctccatggtagagctaggcaagctcatactcacccgccacccttctctgtgcatccacatcctcatcaccaccccgccctaccgcgccaacgacaccgactcatacatcacctccgtctccgctgccaacccttccctcatcttccaccacctccccaccatctccctccctccctccctcgcctcctcccgcaaccacgaaatcctagccttcgaactcgcccccctctacaaccctaacgtccaccaagccctcgtctccatctcccacaacttctccatcaaagcttttgtcatggacttcttctgctatgtcgggctccccgttgccaccgagctgaacatccccagctacttcttcttcacatccagcgccaacaccctcgcttcctccctctacctccccacccttcacaacatcattgacaaaagcctcaaagacctaaatatccttctcaacattccaggagtcccgccgatgccttcctccgatatgccgcaaccgactcttgaccgaaaccaaaaagtgtatgaacatgtccaaggaagctcaaagcagttcccgaaatcagctgggattatcgtaaacacgtttgaatctctcgaacccagagctctcagagcaatatgggacggtctgtgcttgcccgagaacgttccaactccaccggtctaccccatcggaccgctgattgtttcccatggcggtggaggccgcggggccgagtgtttgaaatggctggactcacagccaagtggaagcgtggtgttcctctgttttgggagcttgggattgttttcaaaggagcagttgaaggaaattgcgattgggttggagaatagtgggcacagatttttgtgggtggtccgtaatcctccagcccaaaatcaaattgggctggatattaaagagtccgatccggaattgaaatctttgcttccggacgggtttttggatcggactaaggatcggggtctcgtggtcaagtcatgggccccgcaagcggcagtgttgaatcataactcggtgggtgggtttgtgagtcattgcgggtggaactcggtgttggaatcggtgtgtgccggtgtgccgattgtggcttggccgctctacgcggagcagagattcaatcgagtggttttggtgaaggagattaagattgctatgccgatgaacgagtcagaagacgggtttgtgagagcagcggaggtggagaagcgaattacggagttgatggactcggaggagggcgcgtcgatcaggaagcgtacaaaggatttgcaaaacaatgcccatgcagcattgggtgagaccgggtcgtctggggttgcattgactaaactacttgaattctggggcaaaacctgttag

SEQ ID NO: 14 - DNA, 1425 bp, Malus domestica; identical to SEQ ID NO: 11
  ref <308>: DATABASE ACCESSION NUMBER: GenBank MN381003
  ref <309>: DATABASE ENTRY DATE: 2020-08-07
  ref <313>: RELEVANT RESIDUES IN SEQ ID NO: (1)..(1425)

SEQ ID NO: 15 - DNA, 1425 bp, Malus trilobata
  ref <308>: DATABASE ACCESSION NUMBER: GenBank MN381004
  ref <309>: DATABASE ENTRY DATE: 2020-08-07
  ref <313>: RELEVANT RESIDUES IN SEQ ID NO: (1)..(1425)
atggaggcggcagctatagttttatatccatcaccaccaattggccacttagtctccatggtagagctaggcaagctcatactcacccgccacccttctctgtgcatccacatcctcatcaccaccccgccctaccgcgccaacgacaccgactcatacatcacctccgtctccgctgccaacccttccctcatcttccaccacctccccaccatctccctccctccctccctcgcctcctcccgcaaccacgaaaccctaaccttcggactcgcccccctcaacaaccctaacgtccaccaagccctcctctccatctcccacaacttctccatcaaagcttttgtcatggacttcttctgctctgtcgggctccccattgccaccgagctgaacatccccagctacttcttcttcacatccagcgccaccaccctcgcttccttcctctacctccccaccattcacaacatcactgacaaaagcctcaaagacctaaatatccttctcaacattccaggagtcccgccgattccttcctccgatatgccgcaaccgattcttgaacgaaacaacaaagtgtatgaacagtgccaagaaagctcaaagcagttcccgaaatcagctgggattatcgtaaacacgtttgaatctctcgaacccagagctctcagagcaatatgggacggtctgtgcttgaccgagaacgttccaactccaccggtctaccccatcggaccgctgattgtttcccacggcggtggaggccgcggggccgagtgtttgaaatggctggactcacagccaagtggaagcgtggtgttcctctgttttgggagcttgggattgttttcaaaggagcagttgaaggaaattgcgattgggttggagaatagtgggcacagatttttgtgggtggtccgtaatcctccagcccaaaatcaaattgggctggttattaaagagtccgatccggaattgaaatctttgcttccggacgggttcttggatcggactaaggatcggggtctggtggtcaagtcatgggtcccgcaagtggcagtgttgaatcacaactcggtgggtgggtttgtgagtcattgcgggtggaactcggtgttggaatcggtgtgtgccggtgtgccgattgtgtcttggccgctctacgcggagcagagattaaatcgagtggttttggtggaggagattaagattgctatgccgatgaacgagtctgaagacgggtttgtgagagcagcggaggtggagaagcgagttacggagttgatggactcggaggagggcgagtcgatcaggaagcgtacaaaggatttgcaaaacgatgcccatgcagcattgggtgagaccgggtcgtctggggttgcatttactaaactacttgaattgtggggcaaaactggttag

SEQ ID NO: 16 - DNA, 1425 bp, Malus trilobata
  ref <308>: DATABASE ACCESSION NUMBER: GenBank MN381005
  ref <309>: DATABASE ENTRY DATE: 2020-08-07
  ref <313>: RELEVANT RESIDUES IN SEQ ID NO: (1)..(1425)
atggaggcggcagctatagttttatatccatcaccagtaattggccacttgatcgccatggtagagctaggcaagctcataatcacccgccacccttctctgtgcatccacatcctcatcaccaccccgccctaccgcgccaacgacaccgactcatacatcacctccgtctccgctgccaacccttccctcatcttccaccacctccccaccatctccctccctccctccctcgcctcctcccgcaaccacgaaaccctaaccttcggactcgcccccctcaacaaccctaacgtccaccaagccctcctctccatctcccacaacttctccatcaaagcttttgtcatggacttcttctgctctgtcgggctccccattgccaccgagctgaacatccccagctacttcttcttcacatccagcgccaccaccctcgcttccttcctctacctccccaccattcacaacatcactgacaaaagcctcaaagacctaaatatccttctcaacattccaggagtcccgccgattccttcctccgatatgccgcaaccgattcttgaacgaaacaacaaagtgtatgaacagtgccaagaaagctcaaagcagttcccgaaatcagctgggattatcgtaaacacgtttgaatctctcgaacccagagctctcagagcaatatgggacggtctgtgcttgaccgagaacgttccaactccaccggtctaccccatcggaccgctgattgtttcccacggcggtggaggccgcggggccgagtgtttgaaatggctggactcacagccaagtggaagcgtggtgttcctctgttttgggagcttgggattgttttcaaaggagcagttgaaggaaattgcgattgggttggagaatagtgggcacagatttttgtgggtggtccgtaatcctccagcccaaaatcaaattgggctggttattaaagagtccgatccggaattgaaatctttgcttccggacgggttcttggatcggactaaggatcggggtctggtggtcaagtcatgggtcccgcaagtggcagtgttgaatcacaactcggtgggtgggtttgtgagtcattgcgggtggaactcggtgttggaatcggtgtgtgccggtgtgccgattgtgtcttggccgctctacgcggagcagagattaaatcgagtggttttggtggaggagattaagattgctatgccgatgaacgagtctgaagacgggtttgtgagagcagcggaggtggagaagcgagttacggagttgatggactcggaggagggcgagtcgatcaggaagcgtacaaaggatttgcaaaacgatgcccatgcagcattgggtgagaccgggtcgtctggggttgcatttactaaactacttgaattgtggggcaaaactggttag

SEQ ID NO: 17 - DNA, 1425 bp, Malus ‘Aotea’
  ref <308>: DATABASE ACCESSION NUMBER: GenBank MN381006
  ref <309>: DATABASE ENTRY DATE: 2020-08-07
  ref <313>: RELEVANT RESIDUES IN SEQ ID NO: (1)..(1425)
atggaggcggcagctatagttttatatccatcaccgctaattgggcacttagtctccatggtagagctaggcaagctcatactcacccgccacccttctctgtgcatccacatcctcatcaccaccccgccctaccgcgccaacgacaccgactcatacatcacctccgtctccgctgccaacccttccctcatcttccaccacctccccaccatctccctccctccctccctcgcctcctcccgcaaccacgaaatcctagccttcgaactcgcccccctctacaaccctaacgtccaccaagccctcgtctccatctcccacaacttctccatcaaagcttttgtcatggacttcttctgctatgtcgggctccccgttgccaccgagctgaacatccccagctacttcttcttcacatccagcgccaacaccctcgcttcctccctctacctccccacccttcacaacatcattgacaaaagcctcaaagacctaaatatccttctcaacattccaggagtcccgccgatgccttcctccgatatgccgcaaccgactcttgatcgaaaccaaaaagtgtatgaacatgtccaaggaagctcaaagcagttcccgaaatcagctgggattatcgtaaacacgtttgaatctctcgaacccagagctctcagagcaatatgggacggtctgtgcttgcccgagaacgttccaactccaccggtctaccccatcggaccgctgattgtttcccatggcggtggaggccgcggggccgagtgtttgaaatggctggactcacagccaagtggaagcgtggtgttcctctgttttgggagcttgggattgttttcaaaggagcagttgaaggaaattgcgattgggttggagaatagtgggcacagatttttgtgggtggtccgtaatcctccagcccaaaatcaaattgggctggatattaaagagtccgatccggaattgaaatctttgcttccggacgggtttttggatcggactaaggatcggggtctcgtggtcaagtcatgggccccgcaagcggcagtgttgaatcataactcggtgggtgggtttgtgagtcattgcgggtggaactcggtgttggaatcggtgtgtgccggtgtgccgattgtggcttggccgctctacgcggagcagagattcaatcgagtggttttggtgaaggagattaagattgctatgccgatgaacgagtcagaagacgggtttgtgagagcagcggaggtggagaagcgaattacggagttgatggactcggaggagggcgcgtcgatcaggaagcgtacaaaggatttgcaaaacaatgcccatgcagcattgggtgagaccgggtcgtctggggttgcattgactaaactacttgaattgtggggcaaaactggttag

SEQ ID NO: 18 - DNA, 1425 bp, Malus domestica
atggaggcgacagctatagttttatatccatcacctctaattgggcacttagtctccatggtagagctaggcaagctcatactcacccgccacccttctctgtgcatccacatcctcatcaccaccccgccctaccgtgccaacgacaccgactcatacatcacctccgtctccgccgccaacccttccctcattttccaccacctccccaccatctccctccctccctccctctccccctcccgcaaccacgaaaccccaatcttcgaagtccttctcctcaacaacccttacgtccaccaagccctcctctccatctcccacaacttctccatcaaagcttttgtcatggacttcttctgctctgtcgggctccccattgccaccgagctgaacatccccagctacttcttcttcacatccagcgccgccaacctcgcttgcttcctctacctccccaccattcacagcatcactgacaaaagcctcaaagacctaaatatccttctcaacattccaggagtccagccgattccttcctccgatatgccgaaaccgattcttgaacgaaacaacaaagtgtatgaacatttccaagaaagctcaaagcagttcccgaaatcagctgggattatcgtaaacacgtttgaatctctcgaacccagagttctcagagcaatatgggacggtctgtgcttgacggagaacgttccaactccaccggtctaccccatcggaccgctgattatttcccatggcggtggaggccgcggggccgagtatttgaaatggctggactcacagccaagtggaagcgtggtgttcctctgttttgggagcttgggattgttttcaaaggagcagttgaaggaaattgcgattgggttggagaatagtgggcacagatttttgtgggtggtccgtaatcctccagcccaaaatcaaattgggctggctattaaagagtccgatccggaattgaaatctttgcttccggacgggttcttggatcggactaagggtcggggtctcgtggtcaagtcatgggccccgcaagtggcagtgttgaatcacaactcggtgggtgggtttgtgagtcattgcgggtggaactcggtgttggaatcggtgtgtgccggtgtgccgattgtggcttggccgctctacgcggagcagagattcaatcgagtggttttggtggaggagattaagattgctatgccgatgaacgagtcagaagacgggtttgtgagagcagcggaggtggagaagcgagttacggagttgatggactcggaggagggcgagtcgatcaggaagcgtacaaaggatttgcaaaacgatgcccatgcagcattgggtgagaccgggtcgtctcgggttgcatttactaaactacttgaattctggggcaaaacctgttag